(19) United States
(12) Patent Application Publication
(10) Pub. No.: US 2024/0426662 A1
(43) Pub. Date: Dec. 26, 2024

(54) DETERMINING A LEVEL OF OXYGENATION OF ONE OR MORE CELLS

(71) Applicant: Opticyte, Inc., Seattle, WA (US)

(72) Inventors: Lorilee Arakaki, Bainbridge Island, WA (US); Andrew Carlos Garland, Victoria (CA); Martin Juergen Kellinghusen, Victoria (CA); Kenneth MacCallum, Victoria (CA); Kevin Pierre Meric, Toronto (CA); Luke Aaron Mills, Victoria (CA); Tuyen Kim Nguyen, Victoria (CA); Tristan David Nixon, Victoria (CA); Timothy Sang-Ook Park, Victoria (CA)

(73) Assignee: Opticyte, Inc., Seattle, WA (US)

(21) Appl. No.: 18/821,855

(22) Filed: Aug. 30, 2024

Related U.S. Application Data
(63) Continuation of application No. 17/721,167, filed on Apr. 14, 2022.
(60) Provisional application No. 63/176,014, filed on Apr. 16, 2021.

(51) Int. Cl. G01J 3/10; G01J 3/02; G01J 3/28

(52) U.S. Cl. CPC G01J 3/10; G01J 3/0264; G01J 3/28; G01N 2201/0627

(57) ABSTRACT

An embodiment of a cell-oxygenation monitoring system includes a probe and a base. The probe is connectable to the base, configured to direct electromagnetic energy having wavelengths in an approximate range of 400 nm-900 nm into a body having at least one cell, and configured to receive a portion of the electromagnetic energy redirected by the body during a time. The base includes a generator configured to generate the electromagnetic energy during the time, and a computing circuit configured to determine, in response to the portion of redirected electromagnetic energy, a level of oxygenation of one or more of the at least one cell.

### RELATED PATENTS

This patent application is a continuation of U.S. Utility application Ser. No. 17/721,167 filed Apr. 14, 2022, which claims priority to U.S. Provisional Application Ser. No. 63/176,014 filed on Apr. 16, 2021, the contents of which are incorporated herein in their entirety.

This patent application discloses improvements to, and other differences relative to, the subject matter described in U.S. Pat. No. 9,951,999, titled DETERMINATION OF TISSUE OXYGENATION IN VIVO, filed 2 May 2013, and issued 14 Mar. 2017, and U.S. Pat. No. 10,463,286, titled DETERMINATION OF TISSUE OXYGENATION IN VIVO, filed 10 Feb. 2017, and issued 5 Nov. 2019, which patents are incorporated by reference herein.

### SUMMARY

A system for determining and monitoring a level of oxygenation in one or more cells that form one or more tissues of a body, components of such a system, a method of using such a system, and a method of training a machine-learning algorithm executed by such a system, are described according to one or more embodiments.

Lack of oxygen to one or more vital organs is a complication that an emergency medical specialist (EMS) or a critical care specialist (CCS) (e.g., an emergency-room (ER) doctor, a trauma surgeon) strives to prevent, and, if it occurs, strives to reverse, in a patient who is, for example, critically ill (e.g., has an infection, has had a heart attack) or who is critically injured (e.g., has one or more broken bones, is bleeding internally, is in shock).

Fortunately, there are a number of effective treatments that an EMS or CCS can render to a patient who is experiencing a lack of oxygen to one or more vital organs.

But a key to an EMS or CCS treating such a patient with a high probability of success is the EMS or CCS knowing, with reasonable certainty and within a reasonable time frame, whether the patient's vital organs are adequately oxygenated.

Unfortunately, currently available medical devices and techniques are often unable to determine, with a sufficient level of accuracy and within a sufficient time frame, whether a patient's vital organs are adequately oxygenated. That is, by the time currently available medical devices and techniques determine that a patient's vital organs are inadequately oxygenated, the organs often have been deprived of oxygen for so long that it is too late for the EMS or CCS to save the patient's life or to prevent the patient from experiencing organ dysfunction or failure.

Therefore, if an EMS or CCS who is relying on currently available medical devices and techniques delays treatment until he/she is reasonably sure that one or more of a patient's vital organs is inadequately oxygenated, then he/she runs the risk of one or more of the organs having been deprived of oxygen for so long that the treatment is ineffective to reoxygenate the one or more organs, at least in time to prevent the patient from suffering serious permanent injury or from dying.

Conversely, if, knowing the shortfalls of currently available medical devices and techniques, the EMS or CCS decides not to delay treatment until he/she is reasonably sure that one or more of the patient's vital organs is inadequately oxygenated, then he/she runs the risk that the patient's vital organs are adequately oxygenated and, therefore, that the treatment itself may cause, to the patient, serious illness or injury in addition to the illness or injury that landed the patient in the EMS or CCS's care in the first place.

Consequently, an EMS or CCS relying on currently available medical devices and techniques to determine whether one or more of a patient's vital organs are inadequately oxygenated is often faced with a life-or-death conundrum. If the EMS or CCS waits to treat the patient until he/she is reasonably sure that one or more of the patient's vital organs are inadequately oxygenated, then he/she runs the risk of the patient suffering permanent injury, or even dying, because he/she did not commence the treatment soon enough. But if the EMS or CCS treats the patient before he/she is reasonably sure that one or more of the patient's vital organs are inadequately oxygenated, then he/she runs the risk of the patient unnecessarily suffering significant additional harm.

A pulse oximeter is an example of a currently available medical device (typically worn on a patient's finger) that determines the level of oxygenation, often called the “oxygen saturation,” of a patient's arterial blood. For example, an 85% level of blood-oxygen saturation means that the patient's blood is carrying an amount of oxygen such that 85% of the hemoglobin in the arterial blood is bound to oxygen. A healthy range for a level of blood-oxygen saturation of human blood is typically 98% or greater.

Although a low level of blood-oxygen saturation is, by itself, a good indication that one or more of a subject's vital organs soon will, or already, have inadequate levels of cell-oxygenation, the opposite is not true. That is, a normal level of blood-oxygen saturation typically is not, by itself, a good indicator that one or more of a subject's vital organs have adequate levels of cell oxygenation and are otherwise not being deprived of oxygen. One reason that a normal level of blood-oxygen saturation may not be, by itself, a reliable indicator that all of a subject's vital organs are adequately oxygenated is because there may be times during which a subject's respiratory system is adequately oxygenating the subject's blood, but the subject's circulatory system is supplying inadequate amounts of oxygenated blood to one or more vital organs. For example, an artery or capillaries that supply blood to a vital organ, or a vein or capillaries that drain blood from a vital organ, may become severed, blocked or otherwise restricted, or otherwise damaged, and, therefore, may deprive the organ of oxygenated blood.

Yet another problem with currently available medical devices and techniques for determining and indicating a level of blood-oxygen saturation is that the determined and indicated level of blood-oxygen saturation can exhibit significant error caused by the device or technique being biased against patients having or exhibiting a certain trait.

For example, researchers recently discovered that at least some pulse oximeters exhibit racial bias in their measurements of blood-oxygen-saturation levels, a bias that can result in harm to Black patients. The researchers found that at least some pulse oximeters generate, for Black patients, readings of blood-oxygen saturation that are significantly higher (e.g., up to eight percentage points higher) than the Black patients' actual levels of blood-oxygen saturation. M. W. Sjoding, R. P. Dickson, T. J. Iwashyna, S. E. Gay, T. S. Valley, “Racial Bias in Pulse Oximetry Measurement,” N. Engl. J. Med. 2020, 383 (25): 2477-2478,which is incorporated by reference. As an example, a pulse oximeter may indicate that a Black patient has a level of blood-oxygen saturation of 90% when the actual level of the Black patient's blood-oxygen saturation is 82%. Consequently, an EMS or CCS may delay treatment of a Black patient based on an erroneous pulse-oximeter reading that indicates that the Black patient's level of blood-oxygen saturation is higher than it really is, and this delay may result in the Black patient suffering a serious additional illness or injury, or even may result in the Black patient's death.

But fortunately, it is well known that if a mammalian body cannot adequately oxygenate all of its tissues, then the body prioritizes oxygenating organs over skeletal muscles.

It, therefore, follows (and has been demonstrated) that a level of oxygenation in muscle cells (for example, a level of “cell oxygenation” or a level of “oxygenation” in muscle cells can be defined as a level of “myoglobin oxygen saturation” in the muscle cells), by itself, can be a reliable indicator of whether one or more vital organs soon will be, or already are, inadequately oxygenated. As described above, a body that cannot oxygenate all of its tissues adequately directs blood flow away from skeletal muscles and to vital organs; that is, the body prioritizes blood flow to vital organs over skeletal muscles. So, a normal level of oxygenation in the cells of skeletal muscles can indicate reliably that the body has not directed blood flow away from the skeletal muscles (at least not for a significant length of time), and, therefore, that no vital organ is inadequately oxygenated. In contrast, absent an injury that restricts blood flow to a particular skeletal muscle but that does not reduce blood flow to vital organs, a below-normal level of oxygenation in the cells of the particular skeletal muscle can indicate, reliably, that the body has been directing blood flow away from skeletal muscles, or otherwise has been experiencing a systematic oxygen deficiency that is affecting critical organs and skeletal muscles, and, therefore, that at least one vital organ is receiving an inadequate supply of blood or otherwise is being inadequately oxygenated.

Consequently, a need has arisen for a system configured to measure, to determine, to monitor, and to indicate a level of oxygenation in one or more tissues (e.g., skeletal-muscle tissue) of a subject's body, and to do so with little or no subject-bias, or other, error caused by, or otherwise related to, e.g., the race (e.g., skin tone), age, biological gender, or body-mass index (BMI), of the subject.

An embodiment of a system for determining, monitoring, and indicating a level of oxygenation in one or more cells of skeletal-muscle tissue with little or no subject bias includes a probe and a base. The probe is connectable to the base, configured to direct electromagnetic energy into a body having at least one cell, and configured to receive a portion of the electromagnetic energy redirected by the body, during a time. The base includes a generator configured to generate the electromagnetic energy during the time, and a computing circuit configured to determine, in response to the portion of redirected electromagnetic energy, a level of oxygenation (e.g., a level of myoglobin-oxygen saturation) of one or more of the at least one cell. For example, the system can be configurable or configured to determine, to monitor, and to display (or otherwise to indicate or to provide) a level of oxygenation in one or more cells of skeletal-muscle tissue in the thenar eminence of a human hand.

An embodiment of a probe of such a system includes a head, at least one collector optical fiber, at least one first illuminator optical fiber, at least one second illuminator optical fiber, and a connector. The head is securable to a body having at least one cell, and each of the at least one collector optical fiber has a respective collector end disposed on the head. Each of the at least one first illuminator optical fiber has a respective first illuminator end disposed on the head approximately a first distance from the collector end of at least one of the at least one collector optical fiber, and each of the at least one second illuminator optical fiber has a respective second illuminator end disposed on the head approximately a second distance from the collector end. And the connector is configured for coupling opposite ends of the at least one collector fiber, the at least one first illuminator optical fiber, and the at least one second illuminator optical fiber, to a base configured for determining a level of oxygenation (e.g., a level of myoglobin-oxygen saturation) of the at least one the cell.

An embodiment of a base of such a system includes a generator and a computing circuit. The generator is configured to provide electromagnetic energy to a probe configurable to direct the electromagnetic energy into a body having at least one cell and to collect a portion of the electromagnetic energy redirected by the body over a time during which the generator provides the electromagnetic energy. And the computing circuit is configured to determine, in response to the portion of redirected electromagnetic energy, a level of oxygenation (e.g., a level of myoglobin-oxygen saturation) of one or more of the at least one cell.

An embodiment of a method for indicating a level of oxygenation (e.g., a level of myoglobin-oxygen saturation) includes generating, during an oxygenation-determining time, electromagnetic energy, and determining, in response to a portion of the electromagnetic energy redirected, during the time, by a body having at least one cell, a level of oxygenation of one or more of the at least one cell.

And an embodiment for generating data for training a machine-learning algorithm executed, or otherwise used, by such a system or used in conjunction with such a method includes oxygenating cells in a body part, inducing an ischemia in the body part, returning normal blood flow to the body part, directing, toward the body part, electromagnetic energy, for each of at least one time during the oxygenating, inducing, and returning, generating, during each of the at least one first time for each of at least one wavelength range in a portion of the electromagnetic energy redirected by the body part, a respective value of a characteristic of the at least one wavelength range, and storing the at least one respective value.

### BRIEF DESCRIPTION OF THE DRAWINGS

FIG. 1 is a plot of the electromagnetic-energy absorbances of oxymyoglobin, deoxymyoglobin, oxyhemoglobin, and deoxyhemoglobin, versus wavelength, and a magnified view of the plot in the range of 700 nanometers (nm) to 800 nm, according to an embodiment.

FIG. 2 is a block diagram of a system configured for determining, for monitoring, and for indicating a level of oxygenation in one or more cells of one or more tissues of a body, according to an embodiment.

FIG. 3A is an isometric view of the system of FIG. 2, according to an embodiment.

FIG. 3B is an isometric view of the system of FIG. 2, according to another embodiment

FIG. 4A is a plan view, with portions transparent, of the umbilical cord and the head of the probe of FIG. 2, according to an embodiment.

FIG. 4B is a perspective view of an attachment portion of the probe of FIGS. 2 and 3B attached to a thenar eminence of a subject's hand, according to another embodiment.

FIG. 4C is a perspective view of the probe head of FIGS. 2 and 3B secured to the attachment portion of FIG. 4B, according to an embodiment.

FIG. 4D is a perspective view of a calibrator attached to the probe head of FIGS. 2, 3B, and 4C, according to an embodiment.

FIG. 5 is a flow diagram of a method for using the system of FIG. 2, and of the operation of the system, according to an embodiment.

FIG. 6 is a plan view with portions transparent and other portions broken away, of the array of light-emitting diodes (LED) and the probe-connector receptacle of the base of FIGS. 2-3B, and of the probe connector of the probe of FIGS. 2-3B, according to an embodiment.

FIG. 7 is a bottom plan view of the probe connector of FIGS. 3A, 3B, and 6, according to an embodiment.

FIG. 8 is a rear-end plan view of the probe connector of FIGS. 3A, 3B, 6, and 7, according to an embodiment.

FIG. 9 is a top plan view of the probe connector of FIGS. 3A, 3B, 6, 7, and 8, according to an embodiment.

FIG. 10 is a front-end plan view of the probe connector of FIGS. 3A, 3B, 6, 7, 8, and 9, according to an embodiment.

FIG. 11 is a side view of the electromagnetic-energy blocking member (blocker) of FIG. 6, according to an embodiment.

FIG. 12 is an isometric view, with a portion magnified, of a section of the front end of the probe connector of FIGS. 6, 7, 8, 9, and 10 according to an embodiment.

FIG. 13 is a cutaway side view of the probe-connector receptacle of FIG. 2 without a probe connector installed, and of a clamping mechanism for holding the probe connector of FIGS. 3A, 3B, 6, 7, 8, 9, and 10 within the probe-connector receptacle and for releasing the probe connector, according to an embodiment.

FIG. 14 is a cutaway side view of the probe-connector receptacle and clamping mechanism of FIG. 13 but with a probe connector installed and held within the receptacle, according to an embodiment.

FIG. 15 is a top plan view of a portion of the probe connector of FIGS. 3A, 3B, 7, 8, 9, 10, and 14 with kinematic contact regions labeled, according to an embodiment.

FIGS. 16-18 are respective portions of a flow diagram of a method for collecting training data and for training, with the training data, a machine-learning cell-oxygen-saturation-level-determining algorithm that the base of FIG. 2 is configured to execute, according to an embodiment.

FIG. 19 is a view of an attachment member unattached to a probe head, according to an embodiment.

FIG. 20 is a view of the attachment member of FIG. 19 secured to the thenar eminence of a human hand, according to an embodiment.

FIG. 21 is a view of the attachment member of FIGS. 19 and 20 attached to a probe head while the attachment member is secured to the thenar eminence of a human hand, according to an embodiment.

FIG. 22 is a view of the attachment member of FIGS. 19-21 with a protective film disposed over the attachment member, according to an embodiment.

### DETAILED DESCRIPTION

Unless otherwise indicated, each value, quantity, or attribute herein preceded by “substantially,” “approximately,” “about,” a form or derivative thereof, or a similar term, encompasses a range that includes the value, quantity, or attribute ±20% of the value, quantity, or attribute, or a range that includes ±20% of a maximum difference from the value, quantity, or attribute, or ±20% of the difference between the range endpoints. For example, an “approximate” range of b-c is a range of b−20%·(c−b) to c+20%·(c−b). Furthermore, the terms “a,” “an,” and “the” can indicate one or more than one of the objects that they modify.

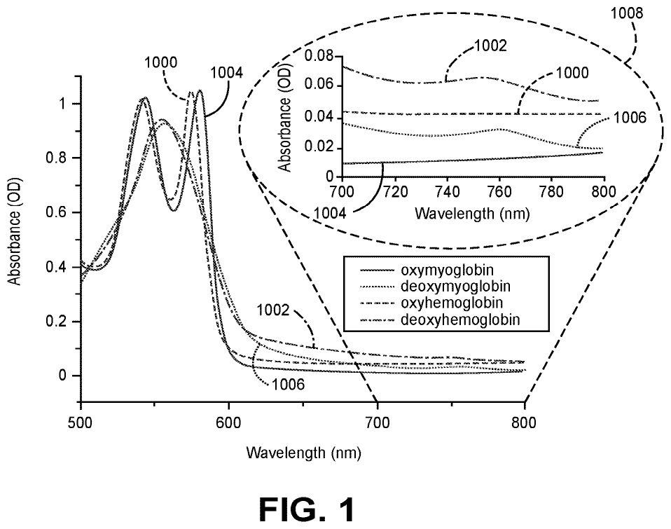

FIG. 1 is an overlay of plots 1000, 1002, 1004, and 1006 of the electromagnetic-energy absorbances of oxyhemoglobin, deoxyhemoglobin, oxymyoglobin, and deoxymyoglobin, respectively, versus wavelength in the approximate range of 500 nm to 800 nm, and includes a magnified view 1008 of the plots in the approximate range of 700 nm to 800 nm, according to an embodiment.

Hemoglobin is the metalloprotein in the red blood cells of all vertebrates. The function of hemoglobin is to transport oxygen in mammalian (e.g., human) blood, and the electromagnetic-energy absorbances of hemoglobin while saturated with oxygen molecules (i.e., while carrying, or while bound with, one or more oxygen atoms) are significantly different than the electromagnetic-energy absorbances of hemoglobin while devoid of oxygen molecules (i.e., not carrying, or not bound with, at least one oxygen atom). Oxygen-saturated hemoglobin is called “oxyhemoglobin”, and hemoglobin devoid of bound oxygen is called “deoxyhemoglobin”.

As described above, a pulse oximeter can measure, or otherwise determine, an overall oxygen-saturation level of all hemoglobin in arterial blood by illuminating blood vessels (through the skin, for example, on a finger) of a subject with electromagnetic energy, measuring the magnitudes of two wavelengths (e.g., 660 nm and 940 nm) of the electromagnetic energy redirected by the blood vessels, effectively comparing the measured magnitudes to magnitudes of the plots 1000 and 1002 at the measured wavelengths, and calculating, in a conventional manner, the level of oxygen-saturation in all hemoglobin in the blood equal to [oxyhemoglobin]/([oxyhemoglobin]+[deoxyhemoglobin])%.

Because a pulse oximeter is typically placed in a region (e.g., fingertip) of a subject's body where there is little or no muscle, and, therefore, little or no myoglobin, even though the electromagnetic absorbance spectra of oxyhemoglobin and oxymyoglobin are similar, as are the electromagnetic absorbance spectra of deoxyhemoglobin and deoxymyoglobin, absorption of the redirected electromagnetic energy by oxymyoglobin or deoxymyoglobin negligibly affects a pulse-oximeter's determination of blood-oxygen-saturation level.

Myoglobin, which is distantly related to hemoglobin, is an iron-and oxygen-binding protein found in the skeletal muscle cells of vertebrates in general, and in almost all mammals; and, like hemoglobin, myoglobin and has significantly different electromagnetic-energy absorbances while saturated with oxygen molecules (i.e., carrying, or bound with, one or more oxygen atoms) than while devoid of oxygen molecules (i.e., not carrying, or not bound with, at least one oxygen atom). Oxygen-saturated myoglobin is called “oxymyoglobin,” and myoglobin devoid of bound oxygen is called “deoxymyoglobin.”

Although the physiological function of myoglobin is not yet conclusively established, it is hypothesized that myoglobin transports at least some oxygen within skeletal muscle cells and stores oxygen within muscle cells.

And, as discussed above, it has been found that low or near-zero oxygen-saturation levels in skeletal-muscle cells resulting from low or near-zero levels of oxymyoglobin in the same tissue can indicate that a subject's body is directing available oxygen away from skeletal muscles and to vital organs, or otherwise is experiencing a systematic oxygen deficiency that is affecting critical organs and skeletal muscles, due to the subject going into, or being in, physical shock due to a catastrophic illness or injury.

Because of the similarities in the absorbance spectra of oxyhemoglobin and oxymyoglobin, and deoxyhemoglobin and deoxymyoglobin, measuring, or otherwise determining, an oxygen-saturation level of myoglobin has been more challenging than measuring, or otherwise determining, an oxygen-saturation level of blood in tissues that do not have muscle and myoglobin (e.g., the finger).

Fortunately, as described below, an embodiment of a system is configured for measuring, or otherwise for determining, an oxygen-saturation level of tissue, such as skeletal-muscle tissue, by taking advantage of the relatively slight differences between the electromagnetic absorbance spectra of oxyhemoglobin and oxymyoglobin, and deoxyhemoglobin and deoxymyoglobin, respectively, particularly in the approximate range of 400 nm-900 nm.

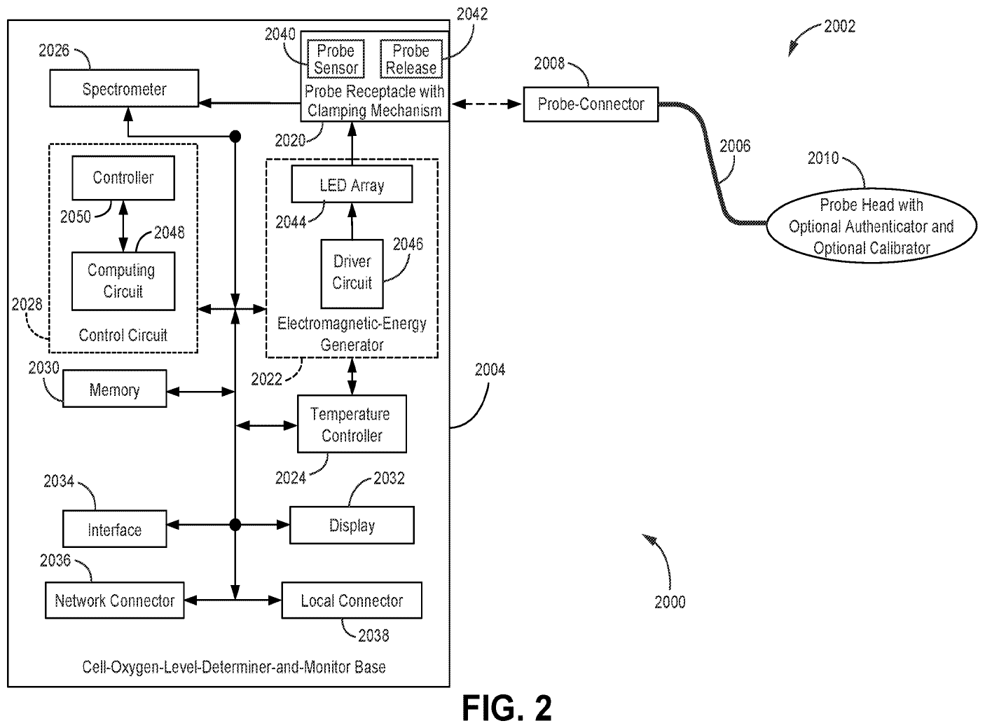

FIG. 2 a block diagram of a system 2000, which is configured for determining, for monitoring, and for indicating a level of oxygenation in one or more cells of one or more tissues of a subject's (e.g., a human's or other mammal's) body (not shown in FIG. 2), according to an embodiment.

The cell-oxygenation-level determining-and-monitoring system 2000 includes a probe (also called a probe unit, probe assembly, or probe apparatus) 2002, and a base (also called a base unit, base assembly, base apparatus, or base device) 2004.

The probe 2002 is configured for receiving, from the base 2004, electromagnetic energy generated by the base, for illuminating, with the received electromagnetic energy, tissue within a subject's body, for receiving, e.g., for collecting, a portion of the electromagnetic energy redirected by one or more cells that form the tissue, and for returning the collected portion of the redirected electromagnetic energy to the base. For example, the probe 2002 can be configured for attaching to and for illuminating a thenar eminence of a human hand, and for collecting, and for returning to the base 2004, a portion of the electromagnetic energy redirected by one or more skeletal-muscle cells of the thenar eminence.

The probe 2002 includes a probe coupling cord, also called an “umbilical cord,” 2006, a probe connector 2008, and a probe head 2010.

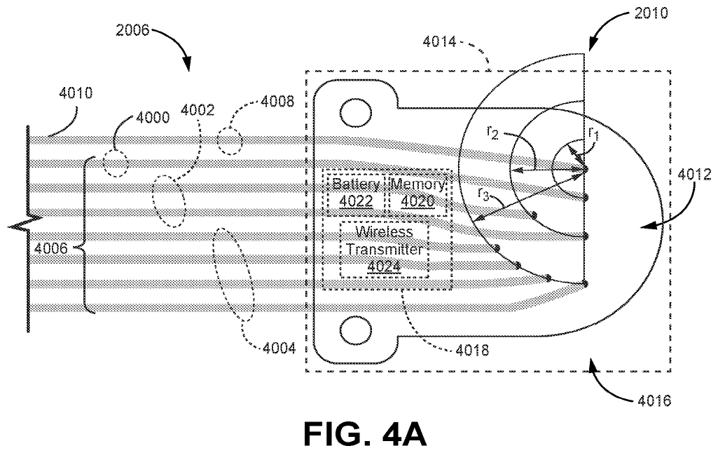

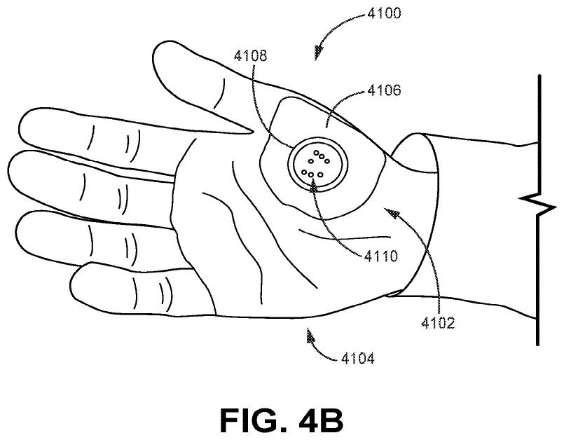

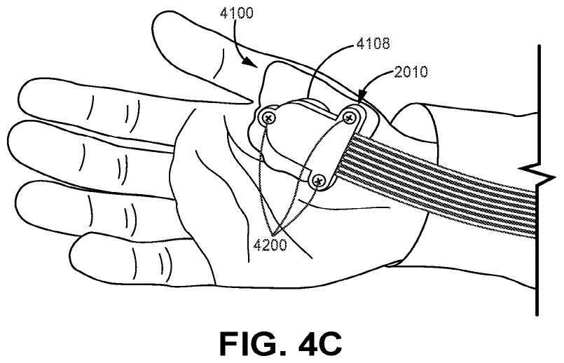

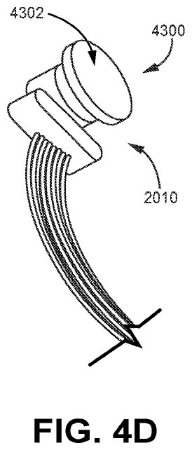

The probe umbilical cord 2006 is flexible and includes at least one illuminator optical fiber (not shown in FIG. 2) configured for delivering illuminating electromagnetic energy from the base 2004 to the head 2010, and at least one collector optical fiber (not shown in FIG. 2) configured for collecting and returning to the base a portion of the illuminating electromagnetic energy redirected by one or more cells that form one or more tissues of a body. Each of the at least one illuminating optical fiber and at least one collector optical fiber may be coated with, or otherwise be contained within, a material opaque to the electromagnetic energy generated by the base 2004 to reduce to a suitable level, or altogether to eliminate, crosstalk between the optical fibers. The probe umbilical cord 2006 is further described below in conjunction with FIGS. 4A, 6, 8, and 12.

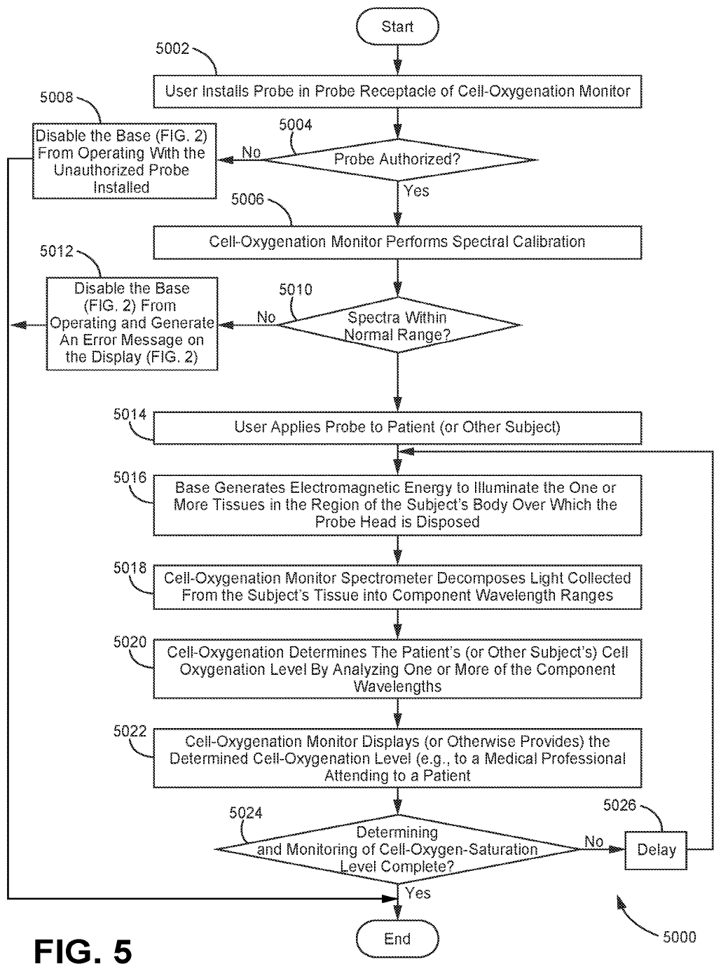

The probe connector 2008 is configured for receiving and holding ends of the at least one illuminator optical fiber (not shown in FIG. 2) and the at least one collector optical fiber (not shown in FIG. 2), and for coupling to a probe receptacle (described below) of the base 2004 such that the end of each of the at least one illuminator optical fiber is stably aligned with a respective portion of an electromagnetic-energy generator (described below), and such that the end of each of the at least one collector optical fiber is stably aligned with a respective input structure of a spectrometer (described below). The probe connector 2008 also includes one or more structures (not shown in FIG. 2) for reducing to a suitable level, or altogether eliminating, cross talk between the at least one illuminator optical fiber and the at least one collector optical fiber. The probe connector 2008 is further described below in conjunction with FIGS. 6-12.

The probe head 2010 includes ends of the at least one illuminator optical fiber and the at least one collector optical fiber opposite the ends disposed within the probe connector 2008, and includes a structure (e.g., an adhesive or a strap) configured for affixing, or otherwise securing, the probe head to a part or portion, such as a thenar eminence, of a body - because the layer of fat and other tissue between the skin and the muscle is relatively and consistently thin and hairless in people of all skin colors, genders, races, ages, and BMIs, measuring cell-oxygenation levels in the tissues of, or near, the thenar eminence reduces measurement bias induced by traits such as skin-tone/race, biological gender, age, and BMI, and, therefore, reduces the magnitude of measurement inaccuracy that may be caused by such measurement bias. The probe head 2010 also can include a calibrator, an authenticator, or both a calibrator and authenticator. The calibrator (further described below in conjunction with FIGS. 4A and 4D) is configured to allow the system 2000 to make and to save a base reading (e.g., a base spectrometer reading), and to adjust the electromagnetic-energy generator (described below) or the cell-oxygenation-saturation-level determination algorithm to account for one or more changes in one or more parameters (e.g., intensity at one or more wavelengths, color, color temperature, wavelength shift) of the electromagnetic energy compared to the most-recent prior cell-oxygenation-saturation-level determination or compared to a baseline electromagnetic spectrum. Alternatively, as described in conjunction with FIG. 5, the calibrator is configured to allow the system 2000 to make and to save calibration values that the system can use later to calibrate, or to normalize, values of an electromagnetic spectrum collected from a subject's tissue by the probe 2002. And the authenticator is configured to provide, to the base 2004, information from which the base can determine that the probe 2002 is authorized for use with the base 2004. For example, the authenticator can prevent use of a probe that is not fully compatible with the base 2004, can prevent use of a same probe 2002 on more than one subject (e.g., beyond a probe's single-use-design limit) to ensure safety and efficacy, and can thwart use of an unauthorized after-market probe that have not been proved to be compatible with the base 2004. Where the authenticator is an electronic device, the authenticator can communicate with the base 2004 wirelessly or via a conductive wire running from the probe head 2010, through the umbilical cord 2006, and to the base 2004, and can receive power from the base over the same or a different conductive wire(s) or can include its own power source such as a battery. Alternatively, the authenticator can be a barcode, a QR code, or other printed code that a user enters into the base 2004 via an interface (described below) such as a barcode scanner. And although shown as being located on the probe head 2010, the authenticator can be located on, or within, the probe connector 2008. The probe head 2010 is further described below in conjunction with FIGS. 4A-4D.

Still referring to FIG. 2, the base 2004 includes a probe receptacle 2020,

electromagnetic-energy generator 2022, temperature-control circuit 2024, spectrometer 2026, computing circuit 2028, memory 2030, display 2032, interface circuit 2034, network connector 2036, and local connector 2038.

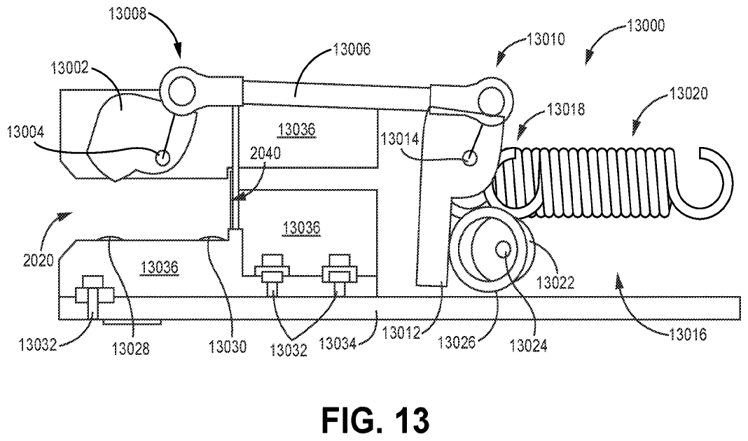

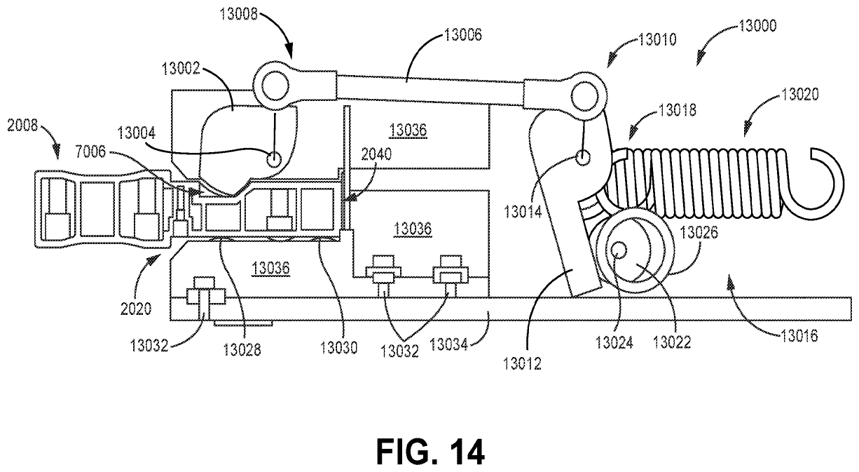

The probe receptacle 2020 is configured to receive, and to hold stably, the probe connector 2008 such that the end of each of the at least one illuminator optical fiber is aligned with a respective electromagnetic-energy source of the generator 2022, and such that the end of each of the at least one collector optical fiber is aligned with a respective input of the spectrometer 2026. The receptacle 2020 includes a sensor 2040 (e.g., a push-button-type microswitch), which is configured to generate a sense signal in response to detecting the presence of the probe connector 2008 in the receptacle, and a clamping mechanism (further described in conjunction with FIGS. 13 and 14) configured, in response the sense signal, to engage and to hold, stably, the probe connector within the receptacle and in optical alignment with the electromagnetic-energy generator 2022 and the input of the spectrometer 2026. For example, the computing circuit 2028 can be configured to receive the sense signal and to activate the clamping mechanism in response to the sense signal. The receptacle 2020 also includes, or is otherwise associated with, a release mechanism 2042, which is configured, when activated, to cause the clamping mechanism to disengage the probe connector 2008 so that one can remove the probe connector from the receptacle. For example, the release mechanism 2042 can be a physical or virtual (e.g., generated by the control circuit 2028 as part of a graphical user interface on the display 2032) push-button-type switch that one presses when he/she wants to disconnect the probe 2002 from the base 2004. The receptacle 2020 is further described below in conjunction with FIGS. 6, 11, 13, and 14.

The electromagnetic-energy generator 2022 is configured to generate a spectrum of wavelengths of electromagnetic energy, for example, in an approximate range of 400 nm-900 nm, such as in an approximate range of 500 nm-800 nm or 550 nm-800 nm, and to direct respective portions of the generated energy into the ends of the at least one illuminator optical fiber within the probe connector 2008. The generator 2022 includes an array 2044 of light-emitting diodes (LEDs shown in FIG. 6) and a driver circuit 2046. The array 2044 is one-dimensional (the LEDs are arranged in a row) and includes, for example, seven LEDs, one LED for each of seven illuminator optical fibers in the probe umbilical cord 2006 and probe connector 2008. Each LED is the same type of LED, for example, an LED configured to emit white light (possibly including light at one or more visible or infrared wavelengths) over an approximate range of 500 nm-800 nm. For example, each LED can include a blue LED that is configured to emit blue light and a yellow phosphor that is configured to absorb some of the blue light and to phosphoresce yellow light in response to absorbing the blue light; the white light radiated by the LED is a combination of the unabsorbed blue light and the yellow light; and the color temperature of the radiated white light depends, at least in part, of the wavelength(s) of the emitted blue light and the wavelength(s) of the phosphoresced yellow light. Furthermore, if commercially feasible, a manufacturer of the system 2000 can form the LED array 2044 with the LEDs formed on a common semiconductor die, or manufactured in a same run or batch, such that the LEDs each radiate electromagnetic energy across a spectrum that is similar, e.g., in one or more of wavelength range, intensity at each wavelength, and polarization at each wavelength, to the spectra generated by the other LEDS in the array. Moreover, the intensity and coherency of the electromagnetic energy that the LEDs luminesce is not dangerous to the human eye; therefore, the base unit 2004 typically will not require a government or industry rating typically given to light-emitting (or other electromagnetic-energy-emitting) devices. In addition, the radiating portion of each LED can be round or cylindrical with a diameter of approximately 1.0 millimeter (mm) or any other suitable size.

The driver circuit 2046 is configured to activate and deactivate each of the LEDs in the array 2044 individually, as subgroups, or as a group, in response to one or more LED-drive-control signals from the control circuit 2028, and, while activating each LED, the driver circuit is configured to provide the LED with a respective stable drive current. An LED is a unijunction (e.g., PN junction) semiconductor device that, while active, develops across the junction a relatively constant voltage in the approximate range of 1.6 Volts (V) to 4.4 V, or in an approximate range of 2.5 V to 3.25 V (“relatively constant” accounts for slight changes that can occur in the junction voltage due to temperature and the magnitude of the forward current through the LED). Consequently, the driver circuit 2046 is configured to activate each of the LEDs in the array 2044 by driving the LED with a respective stable (typically regulated) drive current having a magnitude that causes the LED to radiate (or “to luminesce”) electromagnetic energy with a corresponding level of intensity. That is, to cause the LED to luminesce at a particular level of intensity, the driver circuit 2046 is configured to drive the LED with a current having a magnitude that corresponds to the particular level of intensity. For example, the driver may include, for each LED in the array 2044, a respective current source configured for driving the LED at a stable current having a magnitude corresponding to the particular level of intensity at which the LED is configured to luminesce while the LED is active, or “on.” During operation, the driver circuit 2046 drives the LED with a stable “on” current to turn the LED “on” such that the LED luminesces at an intensity related to the magnitude of the “on” current, and drives the LED with a stable “off” current to turn the LED “off” such that the LED luminesces at an intensity related to the magnitude of the “off” current, which may approximately equal zero or is at least small enough to cause the LED to luminesce at little to no intensity.

The driver circuit 2046 is configured to drive each of the LEDs in the array 2044 with an approximately same drive current so that each LED luminesces an approximately same spectrum of electromagnetic radiation; at least in some cell-oxygen-saturation-level-determining applications it has been found that multiple LEDs luminescing an approximately same spectrum of electromagnetic radiation yields more accurate cell-oxygen-saturation-level readings than multiple LEDs of which one or more of the LEDs luminesce different spectra than one or more of the other LEDs.

As an LED ages, it may tend to luminesce electromagnetic energy at a lower intensity for a given magnitude of drive current; therefore, the generator 2022 may include circuitry configured to cause the driver circuit 2046 to increase the drive currents to the LEDs as the LEDs age so as to maintain the intensity of the electromagnetic energy that each LED generates at an approximately constant level over a long period of time and at an approximately same level relative to the other LEDs.

Furthermore, for a given drive current, the intensity, or other characteristics (e.g., spectrum width, intensity at one wavelength relative the intensity at another wavelength, polarization) of electromagnetic energy that an LED luminesces can change with temperature.

Therefore, the temperature controller 2024 is configured to maintain, at an approximately constant uniform temperature, each of the LEDs in the array 2044. The controller 2028 can command, or otherwise control, the temperature controller 2024 to maintain each of the LEDs at a particular temperature so that the intensity or one or more other characteristics of the electromagnetic energy that the LED luminesces experiences little or no shift with changes in the ambient temperature inside of the base 2004. Said another way, the control circuit 2028 is configured to control the temperature controller 2024 such that the temperature controller minimizes the slope (ideally a slope of zero) of a temperature gradient across the LEDs. The temperature controller can include one or more of a fan, a heat sink, a thermoelectric cooler, a pumped-liquid cooler, a compressed-fluid heat pump configured to heat or cool, and a resistive, or other type of, heater.

The spectrometer 2026 can be an off-the-shelf spectrometer and is configured to separate the tissue-redirected electromagnetic energy collected by the one or more collector optical fibers of the probe 2002 into ranges of wavelengths and to generate, for each wavelength range, at least one signal having a characteristic that represents, or that is otherwise related to, a characteristic of the wavelengths in the range. For example, the spectrometer 2026 can be configured to generate an analog or digital signal having a magnitude that is related to the intensity of the wavelengths within a respective wavelength range. The spectrometer includes a structure having a diffraction grating between two focusing mirrors (not shown in FIG. 2), the structure configured to generate spatially separated ranges of wavelengths (much like a conventional prism generates spatially separated colors of visible light) that are incident on respective portions of a CMOS (or other) electronic pixel array (not shown in FIG. 2), which can be similar to a pixel array included in a smart phone or digital camera. Each portion of the pixel array on which is incident the electromagnetic energy of a respective wavelength range converts the incident energy into a respective electronic signal having a characteristic (e.g., magnitude, phase) that is related to a characteristic (e.g., intensity) of the incident energy. And the spectrometer 2026 provides the signals (a respective at least one signal for each wavelength range) to the control circuit 2028 for processing. The spectrometer 2026 also can include more optics, such as an optical train, between the spectrometer's optical input and the collected-energy output of the receptacle 2020 or of the probe connector 2008.

The control circuit 2028 is configured (e.g., by one or more of software, firmware, and a data stream) to control the configuration and operation of one or more other components of the base 2004 and includes a computing circuit 2048 and a controller 2050. Each of the computing circuit 2048 and controller 2050 can include a respective one or more of a microprocessor, microcontroller, and a field-programmable gate array (FPGA), or the computing circuit and controller can be disposed on a same set of one or more of a microprocessor, microcontroller, and an FPGA. And the computing circuit 2048 can be configured to execute an algorithm, such as a machine-learning algorithm, to determine, in response to the one or more signals from the spectrometer 2026, a cell-oxygen-saturation level of one or more cells in tissue of a subject illuminated by the probe head 2010.

The control circuit 2028 also can include internal memory (not shown in FIG. 2), such as cache memory, working memory, other volatile memory, and nonvolatile memory, which internal memory is in addition to the memory 2030, which is external to the control circuit.

The memory 2030 is configured to store data, and can include one or more of any suitable types of volatile memory and nonvolatile memory. Types of data that the memory 2030 can be configured to store include operating-system and application-program instructions for execution by the control circuit 2028, firmware, a respective data stream for each of one or more FPGAs that may form part of the control circuit 2028, machine-learning-algorithm training data, cell-oxygenation-saturation levels determined by the control circuit, and still-image or video data formatted for rendering on the display 2032. Examples of volatile memory include static-random-access memory (SRAM) and dynamic-random-access memory (DRAM), and examples of nonvolatile memory include read-only memory (ROM), electrically programmable read-only memory (EPROM), electrically programmable-and-erasable read-only memory (EEPROM), flash memory, ferro-electric random-access memory (FeRAM), a magnetic disk, and an optical disk.

The display 2032 is configured to display, for example, a cell-oxygenation-saturation level most recently determined or updated by the computing circuit 2048, a status of the system 2000, an error message such as the probe 2002 is unauthorized for use with the base unit 2004, and a menu of options such as system set up. The display 2032 can include, for example, one or more of a liquid-crystal display, organic LED (OLED) display, dot-matrix display, or any other suitable display. Furthermore, the display 2032 can be a touch-screen display that allows, for example, an EMS OR CCS to select a subset of one or more menu options from a set of displayed menu options of a graphical user interface.

The interface 2034 is configured to allow an EMS OR CCS or other user to input information to the system 2000, and to output information from the system. For example, for input of information, the interface 2034 can include one or more of a keyboard, mouse, microphone (e.g., for example, to input voice information that is converted to data by voice-transcription software running on the computing circuit 2048), barcode or QR code scanner (e.g., for scanning an authentication code on the probe 2002), or the interface can include the display 2032 if the display is a touch screen. And for output of information, the interface can include one or more of a speaker, a transmitter such as a Bluetooth adapter (e.g., to transmit information to a smart phone), one or more lights (e.g., LEDs separate from the LEDs in the array 2044) and a display such as the display 2032.

The network connector 2036 is configured to allow the system 2000 to communicate with another device or system, for example to communicate with a remote server (e.g., a cloud server) over the internet. The network connector 2036 can include, e.g., one or more of a WiFi® adapter, an ethernet adapter and connector, a Bluetooth® adapter, or other wireless adapter or connector. Furthermore, a remote server or other device can send algorithm, software, firmware, and other updates to the control circuit 2028 via the network connector 2036, and the control circuit 2028 can send the status of the system 2000, spectrum data collected from a subject, and other data to a remote server or other device via the network connector.

And the local connector is configured to allow the system 2000 to communicate with another local device or system, such as one or more other electronic medical devices connected to a patient, such as a heart monitor. For example, the local connector can be a wireless adapter or a universal-serial-bus (USB) connector.

Still referring to FIG. 2, alternate embodiments of the system 2000 are contemplated. For example, the umbilical cord 2006 can be permanently connected to the base 2004 and the probe connector 2008 can be configured to receive the probe head 2010. Furthermore, the generator 2022 can include one or more electromagnetic-energy sources in addition to, or other than, LEDs. Moreover, one or more of the computing functions of the computing circuit 2048, such as determining a level of cell-oxygenation in response to signals generated by the spectrometer 2026, can be performed by a computing circuit (e.g., a cloud server) remote from the base 2004. In addition, the control circuit 2028 can generate, on the display 2032, a graphical user interface that allows a user to input commands such as starting a cell-oxygen-saturation-level-determining cycle after the probe 2002 is connected to, and authorized by, the control circuit. Furthermore, although described as remaining open while no probe connector 2008 is installed therein, the probe receptacle can have a spring-loaded flap or shutter to close or seal the receptacle (e.g., to prevent dirt or other substances from entering into the receptacle) while no probe connector is installed therein. Moreover, although described as sliding or “plugging” into the receptacle 2020, the probe connector 2008 and the receptacle can be threaded such that the probe connector is configured for screwing into and out of the receptacle. In addition, although described as a standalone device, the base 2004 can be integrated with one or more other devices such as a heart monitor or automatic external defibrillator (AED). Furthermore, after the control circuit 2028 activates one or more sets of the LEDs of the LED array 2044, the control circuit can determine whether the signals from the spectrometer 2026 indicate that the collector optical fiber 4010 is receiving any redirected electromagnetic energy. If the control circuit 2028 determines that the collector optical fiber 4010 is receiving little or no redirected electromagnetic energy (e.g., the magnitudes of some or all of the signals from the spectrometer 2026 are below a threshold), then the control circuit determines that the probe head 2010 is not properly secured to the subject, deactivates the LEDs so that no electromagnetic energy emanates from the probe head, and sounds (via a sound generator, like a speaker, that is part of the base 2004 but is not shown in FIG. 2), or generates on the display screen 2032, an alarm or other notification informing the user to secure the probe head to the subject. In addition, instead of being of a same type, the LEDs can be of different types, for example each of one or more of the LEDs can be configured to radiate a respective spectrum that is different than the spectrum radiated by the others of the one or more the LEDs. Moreover, although described as having a diameter of approximately 1.0 mm, each LED can have any respective suitable dimensions such as having a larger or smaller diameter. Furthermore, instead of a diffraction grating between two focusing mirrors, the spectrometer 2026 can include another wavelength-separating structure such as a prism. In addition, instead of the electromagnetic-energy generator 2022 and the spectrometer 2026, the base 2004 can include an electromagnetic-energy generator configured to generate a group of one to a few wavelengths at any one time, and to sweep the one to a few wavelengths through an approximate range of, for example, 500 nm-850 nm, and can include a photo detector. Because the control circuit 2028 “knows” what one to a few wavelengths the generator is generating at any one time, a broad-band photo detector can detect the, e.g., intensity, of the one to a few wavelengths and the control circuit can generate a correspondence between each detected intensity (or other detected characteristic(s) of the energy) and the one to a few wavelengths that generated the detected intensity (or other characteristic) to mimic the operation of the spectrometer 2026. Or, instead of a generator configured to generate one to a few wavelengths at any given time and to sweep the one to a few wavelengths, the generator can include a broadband source and a monochromator having a wavelength passband that is swept through a similar wavelength range so, as in the swept-energy-generator example, the photodetector detects an intensity (or other characteristic) of a known group of one or more wavelengths at any given time. In another embodiment, the electromagnetic-energy generator 2022, or a portion thereof (e.g., the LED array 2044) may be disposed in the probe 2002 instead of in the base 2004. Moreover, embodiments described in conjunction with FIGS. 1 and 3A-18 may be applicable to the cell-oxygen-saturation-level determining system 2000 of FIG. 2.

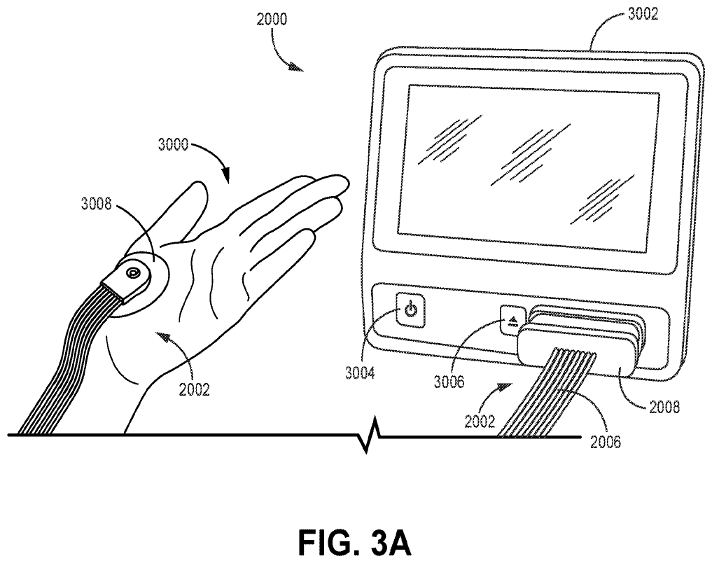

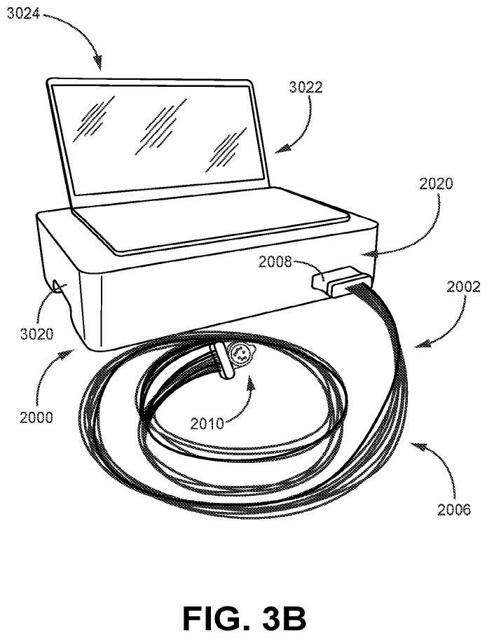

FIG. 3A is a perspective view of the cell-oxygenation-level determining-and-monitoring system 2000 of FIG. 2, according to an embodiment, and of a human hand 3000.

Referring to FIGS. 2 and 3A, the system 2000 includes a housing 3002 and the probe 2002.

Within the housing 3002 are disposed the probe receptacle 2020, electromagnetic-energy generator 2022, temperature controller 2024, spectrometer 2026, control circuit 2028, memory 2030, interface 2034, network connector 2036, and local connector 2038, and mounted to the housing is the display 2032, a system power on/off control (e.g., push-button switch) 3004, and a probe-connector release (e.g., push-button switch) 3006. The housing 3002 can be formed from any suitable material such as injection-molded plastic or metal and can have any suitable shape and dimensions. For example, the housing 3002 can be shaped, and otherwise can be configured, for mounting to a pole as part of a “tree” of medical devices (e.g., heart-rate and blood-pressure monitors) as often can be found in a patient's hospital room or other setting.

The umbilical cord 2006 is an optical-fiber ribbon cable, and the probe 2002 includes an attachment member 3008 configured for attaching (e.g., by an adhesive) to a body part (e.g., the thenar eminence of a human hand) and to which the probe head 2010 is configured to attach. Furthermore, the probe 2002 can include a separate calibrator (FIG. 4D) that one can attach to the probe head 2010 after connecting the probe to the base 2004 but before attaching the probe head to the attachment member 3008 so that the controller 2028 can execute a probe-calibration procedure.

In addition to causing the display 2032 to render the determined cell-oxygenation level, the controller 2028 can be configured to cause the display to render, e.g., a plot of cell-oxygenation level over time, and a graphical user interface that allows a user, e.g., to configure the system 2000, and to enter commands (e.g., release the probe connector 2008 if the release 3006 is omitted) via the interface 2034, which can include the display 2032 if the display is a touch-screen display.

Still referring to FIG. 3A, alternate embodiments of the system 2000 are contemplated. For example, the system 2000 can be configured to “travel with the patient;” that is, the system 2000 can be configured to be carried and used on a patient by first responders, and then to be “handed off” with a patient at the hospital such that there is no period of time during which the cell-oxygenation level of the patient is not being monitored. In such a configuration, the system 2000 can include a dual power supply and a compartment for a battery such that the system can be powered by a wall power outlet while the patient is in a hospital and by a battery while the patient is in transit (e.g., by ambulance or helicopter) to the hospital, between hospitals, or between different areas (e.g., operating room, intensive care unit (ICU)) of a hospital. Furthermore, even a system 2000 that is not configured to “travel” with the patient can include a compartment for a backup battery that the system taps to maintain power to the system in the event of, e.g., power outage or a power cord of the base station 2004 being accidentally “unplugged.” And the system 2000 can include circuitry for charging the battery. In addition, embodiments described in conjunction with FIGS. 1-2 and 3B-18 may be applicable to the cell-oxygenation-level determining system 2000 of FIG. 2.

FIG. 3B is a perspective view of the cell-oxygenation-level determining-and-monitoring system 2000 of FIG. 2, according to another embodiment.

Referring to FIGS. 2 and 3B, the system 2000 includes a housing 3020, the probe 2002, and a computing machine, such as a laptop computer 3022.

One or more of the components of the system 2000 are disposed within the housing 3020, and one or more of the components of the system are disposed within the laptop 3022 (or the functions performed by the one or more components are performed by the laptop). For example, within the housing 3020 are disposed the probe receptacle 2020, electromagnetic-energy generator 2022, temperature controller 2024, spectrometer 2026, and local connector 2038, and within the laptop 3022 are disposed the control circuit 2028, memory 2030, interface 2034, network connector 2036, power on/off control 3004, and probe-connector release control 3006 (or the functions of these components are performed by the laptop), and mounted to the laptop is a display 3024, which can function as the system display 2032. For example, the laptop computer 3022 can be coupled to the system components within the housing 3020 via the local connector 2038, which, as described above, can be a USB connector.

The housing 3020 can be formed from any suitable material such as injection-molded plastic or metal and can have any suitable shape and dimensions. For example, the housing 3020 can be shaped, and otherwise can be configured, to allow mounting, or otherwise to allow disposing, the laptop 3022 on top of the housing.

The probe 2002 can include a separate calibrator (FIG. 4D) that one can attach to the probe head 2010 after connecting the probe to the receptacle 2020 but before attaching the probe head to an attachment member, such as the attachment member 3008 of FIG. 3A, so that the controller 2028 can execute a probe-calibration procedure (described in conjunction with FIG. 5). Furthermore, the umbilical cord 2006 includes a bundle of one or more individual illuminator optical fibers (FIG. 4A) and one or more individual collector optical fibers 4010 (FIG. 4A).

In addition to causing the display 3024 to render the determined cell-oxygenation level, the controller 2028 can be configured to cause the display 3024 to render, e.g., a plot of cell-oxygenation level over time, and a graphical user interface (GUI) that allows a user, e.g., to configure the system 2000, and to enter commands (e.g., release the probe connector 2008) via the interface 2034, which can include the display 3024 if the display is a touch-screen display.

Still referring to FIG. 3B, alternate embodiments of the system 2000 are contemplated. For example, if the system includes a separate calibrator such as described below in conjunction with FIG. 4D, the separate calibrator can be removably, or otherwise, attached, e.g., to the housing 3020. Furthermore, embodiments described in conjunction with FIGS. 1-3A and 4A-18 may be applicable to the cell-oxygenation-level determining-and-monitoring system 2000 of FIG. 2.

In another alternative embodiment, the functionality of the system 2000 can be integrated into a single, integrated unit instead of into a base unit and a probe unit. For example, such an integrated unit can include sensors and circuitry configured to perform the functions of the probe 2002 components (the probe head 2010, umbilical cord 2006, and probe connector 2008), the electromagnetic-energy generator 2022, and the control circuit 2028 in a single housing that is configured to be attached to the thenar eminence of a human hand or to another body part. In more detail according to an embodiment, the integrated unit can include an electromagnetic unit configured to direct electromagnetic (e.g., optical) energy into a part of the body such as the thenar eminence and to receive a portion of the electromagnetic energy that is redirected by the body. An optical sensor, such as the spectrometer 2026, in the integrated unit is configured to convert the redirected portion from an electromagnetic signal to an electrical signal. A computing circuit in the integrated unit, such as the computing circuit 2048, is configured to determine a level of oxygenation of the muscle cells based on the electrical signal. In addition, the integrated unit can include a housing that includes one or more components of the system 2000, including the components of the base 2004 and components that mimic the functionality of the probe 2002. The housing can be of suitable dimensions (e.g., a clip-on housing similar to a clip-on pulse-oximeter housing) to attach directly to the thenar eminence or other part of the subject's body. When implemented as an integrated unit, some components of the system 2000 may not be required, such as the probe 2002, umbilical cord 2006, and the connector 2008.

FIG. 4A is a top plan view of the umbilical cord 2006 and of the probe head 2010 of FIGS. 2 and 3B, according to an embodiment.

The probe head 2010 includes the ends of three sets 4000, 4002, and 4004 of illuminator optical fibers 4006, the ends of one set 4008 of collector optical fibers 4010 (only one collector optical fiber included in the described embodiment), an adhesive bottom surface 4012 configured for sticking to a thenar eminence of a subject's hand, a removable, flexible plastic backing or strip 4014 for covering and protecting the adhesive bottom surface and including a calibration surface 4016 facing the adhesive bottom surface, and an authenticator circuit 4018.

The ends of each set 4000, 4002, and 4004 of illuminator optical fibers 4006 are positioned along a respective arc so that the ends of a set are approximately a same radial distance from the ends of the one or more collector optical fibers, here one collector optical fiber 4010. The end of the optical fiber 4006 in the set 4000 is a radial distance r₁ from the end of the collector optical fiber 4010, the ends of the optical fibers in the set 4002 are each a radial distance r₂ from the end of the collector optical fiber, and the ends of the optical fibers in the set 4004 are each a radial distance r₃ from the end of the collector optical fiber. For example, r₁˜2 mm, r₂˜7 mm, and r₃˜12 mm.

Illuminating a subject's tissue, such as the tissue forming the thenar eminence, from more than one distance can generate information from which the computing circuit 2048 can better determine the oxygen-saturation level of one or more cells that form the tissue as compared to illuminating from only one distance. It has been found that the farther the end of an illuminator optical fiber 4006 to the collector end of a collector optical fiber 4010, statistically the more deeply the illuminating “particles” (e.g., photons) penetrate into the tissue before the tissue redirects the photons to the collector end of the collector optical fiber. By including multiple distances (three distances in the described embodiment) between the illuminator optical fibers 4006 and the collector optical fiber 4010, the probe head 2010 is configured to collect, and to provide to the base 2004 (FIG. 2), spectral information from multiple layers of tissue. For example, energy emanating from the distance r₁ and redirected to the collector end of the collector optical fiber 4010 provides information regarding the uppermost one or more layers (e.g., skin, fat) of the subject's tissues. Energy emanating from the distance r₂ and redirected to the collector end of the collector optical fiber 4010 provides information regarding middle layers (e.g., the uppermost one or more layers of skeletal muscle) of the subject's tissues. And energy emanating from the distance r₃ and redirected to the collector end of the collector optical fiber 4010 provides information regarding lower layers (e.g., the mid to lower layers of skeletal muscle) of the subject's tissues. Collecting spectral information regarding multiple layers of the subject's soft tissues effectively allows the computing circuit 2048 (FIG. 2) to distinguish oxymyoglobin, deoxymyoglobin, oxyhemoglobin, and deoxyhemoglobin from one another and from other substances.

Furthermore, the larger the radial distance, the more illuminator optical fibers 4006 in the set so that for a given illumination time, the level (e.g., the number of “particles,” such as photons) of electromagnetic energy that the tissue redirects to the end of the collector optical fiber 4010 over a given collection time window or period is approximately the same for each radial distance (e.g., r₁, r₂, and r₃). For any given set of radial distances, r₁, r₂, . . . , rₙ, one can determine the number of illuminating optical fibers 4006 needed at each radial distance such that the level of electromagnetic energy redirected to the end of the collecting optical fiber 4010 from each radial distance is approximately the same given that each illuminating optical fiber emanates an approximately same intensity of electromagnetic energy for an approximately same time as each of the other illuminating optical fibers.

The adhesive on the probe surface 4012 can be any suitable adhesive, such as an adhesive used to secure electrocardiogram (EKG) electrodes or AED electrodes to a subject's skin.

The calibration surface 4016 can be any surface suitable for use in calibrating the cell-oxygenation-level determiner-and-monitor system 2000 (FIG. 2), for example, every time a new probe 2002 is installed, which is typically for each new subject. The calibration surface 4016 is highly white reflective surface having a suitable color temperature, which is selected according to the one or more wavelengths in the spectrum of electromagnetic energy generated by the generator 2022.

Before a medical professional, such as a nurse, removes the flexible plastic strip 4014, he/she installs the probe connector 2008 (FIGS. 2 and 3B) into the probe receptacle 2020 (the strip, or other portion of the probe 2002, may include a printed instruction not to remove the strip until after the probe is installed and after the control circuit 2028 generates, on the display 2032, an indication that it is ok to remove the strip).

In response to sensing the probe connector 2008 (FIG. 2) in the receptacle 2020 and that the probe connector is being held in place by the clamping mechanism (FIGS. 13A and 13B), and confirming, by communication from the authenticator 4018, that the probe 2002 is authorized for use with the system 2000, the control circuit 2028 causes the generator 2022 to “flash” the LEDs in the array 2044 for a calibration period of time. During the calibration time, electromagnetic energy propagates along the illuminator optical fibers 4006 and exits the end of these fibers. The calibration surface 4016 redirects a portion of the electromagnetic energy into the end of the collector optical fiber 4010, and the spectrometer 2026 spatially separates the wavelengths of the redirected electromagnetic energy into ranges as described above, and, for each wavelength range, generates a respective calibration value corresponding to the combined intensity of the wavelengths in the range. The control circuit 2028 then stores the one or more calibration values.

After the calibration procedure is complete, the control circuit 2028 can generate, on the display 2032, or with a sound generator, a notification that the nurse or other medical professional can remove the plastic strip 4014 and affix the probe head 2010 to the subject.

Alternatively, the calibration procedure may be so quick that as long as the clinician “plugs” the probe 2002 into the receptacle 2020 before removing the plastic strip 4014, he/she can remove the strip at any time thereafter because the calibration procedure will finish in a time before even the fastest person could remove the plastic strip. Thereafter (until the execution of a subsequent calibration procedure), for each measurement of the wavelength-range intensities, the computing circuit 2048 generates, for each wavelength range, a normalized intensity value equal to the log₁₀ of the ratio of the calibration value over the currently measured value. It is the normalized intensity values for the one or more wavelength ranges that the computing circuit 2048 uses in determining the oxygen-saturation level of the cells in the illuminated tissue. Such calibration can account for changes and differences from base 2004 to base that may occur over time; examples of such base-to-base changes include a change or difference in the respective brightness of, or the respective spectrum generated by, each of one or more of the LEDs in the array 2044, where the change or difference is due to a respective change or difference in one or more the driver circuit 2046, optical coupling (e.g., at the probe head 2010 or at the probe receptacle 2020), and spectrometer 2026. The calibration procedure is further described below in conjunction with FIG. 5.

The authenticator circuit 4018 is configured to provide, to the base 2004 (FIG. 2), information from which the control circuit 2028 (FIG. 2) can determine whether the probe 2002 is authorized for use with the base 2004 (e.g., to prevent unauthorized after-market probes from being used and to promote safety by ensuring that an otherwise authorized probe is fully compatible with the base and is not used beyond its single-use design limit). For example, the authenticator circuit 4018 can include a memory 4020, a battery 4022, and a wireless transmitter (e.g., a Bluetooth® transmitter including a suitable antenna) 4024 or can include a conventional radio-frequency-identifier (RFID) tag or near-field-communication (NFC) circuit. The memory 4020 is a suitable nonvolatile memory, such as a read-only memory (ROM), electrically programmable read-only memory (EPROM), or an electrically programmable-and-erasable read-only memory (EEPROM), that stores an authentication value, which can be coded; for example, the memory can be programmed to store the authentication value by the manufacturer of the probe 2002 (FIG. 2) before the probe is packaged and shipped for sale. The memory 4020 can be constructed in a conventional manner to prohibit one (e.g., a hacker) from thereafter reading the authentication value from the memory. The battery 4022 is suitably sized to fit on the probe head 2010 and is configured to source power to the memory 4020 and transmitter 4024. To extend the life of the battery 4022 while the probe 2002 is “sitting on a shelf” awaiting use, an electrical insulator, such as a plastic strip, may be disposed between a contact electrode of the battery and the power-supply node of circuitry onboard the probe head 2010 to prevent the circuitry from drawing current from the battery, and the probe can include instructions printed thereon (or on the electric insulator) for a user to remove the insulator, thus allowing a power-supply node to make electrical contact with the battery electrode so that the circuitry can draw power from the battery. The transmitter 4024 is configured to send the authentication value to the base 2004 via the network connector 2036 (FIG. 2) or the local connector 2038 (FIG. 2). For example, if the wireless transmitter is a Bluetooth® transmitter, then the transmitter can pair with the base 2004 in a conventional manner. Furthermore, the transmitter 4024 can be configured to code the authentication value before transmission so that one cannot intercept the transmitted value and use it to enable use of third-party or counterfeit probes with the base 2004. The control circuit 2028 (FIG. 2) is configured to receive, and, if needed, to decode, the received authentication value. If the control circuit 2028 determines that the authentication value indicates that the probe 2002 is authorized for use with the base 2004, then the control circuit maintains the base in a functional mode of operation. Conversely, if the control circuit 2028 does not receive an authentication value, or receives an invalid authentication value, then the control circuit disables the base from being used until an authorized probe 2002 is connected to the base via the receptacle 2020. The authentication feature, therefore, can prevent third-party or counterfeit probes from being used with the base 2004. Alternately, the battery can be omitted and the probe 2002 can include one or more wires that connect to power terminals in the receptacle 2020 (FIG. 2) so that the transmitter and memory are powered by the base 2004. And, in another alternative where the authenticator circuit 4018 is, or includes, an RFID tag or NFC circuit, the battery 4022 can be omitted and the authenticator circuit can be powered by a polling or other signal transmitted by the base 2004 (which would include an RFID or NFC reader circuit).

Still referring to FIG. 4A, the illuminator and collector optical fibers 4006 and 4010 can be any suitable type of optical fibers. For example, each fiber 4006 and 4010 can have an opaque outer sheathing to reduce, or altogether eliminate, cross talk between the fibers, particularly between an illuminator optical fiber and a collector optical fiber. Furthermore, although each of one or more the fibers 4006 and 4010 can be a respective glass fiber, it also can be plastic fiber, for example, a multicore plastic fiber. A plastic optical fiber is typically less expensive than a glass optical fiber, and a multicore plastic optical fiber can have a smaller minimum-bend radius than a single-core glass or plastic fiber. The smaller the bend radii of the optical fibers 4006 and 4010, the lower the height of the probe head 2010 can be. In addition, the collector optical fiber 4010 can include multiple collector cores, where each of one or more of a first set of the collector cores is configured to provide collected electromagnetic energy to the spectrometer 2026 as described elsewhere in this application, and where each of one or more of a second set of the collector cores is configured to provide, while the probe head 2010 is properly attached to a subject's body part, light reflected from the body part to a detection circuit (e.g., photodetector) onboard the base 2004. The control circuit 2028 can be configured to “recognize” the presence of reflected light as an indication that the probe head is properly attached to a body part. Consequently, to prevent the electromagnetic-energy generator 2022 from activating the LEDs while the probe head 2010 is not properly attached to a subject, the control circuit 2028 can be configured to enable operation of the base 2004 in response to detecting at least a threshold intensity level of reflected light from the second set of one or more collector cores, and to disable operation of the base otherwise.

Still referring to FIG. 4A, alternate embodiments of the probe head 2010 are contemplated. For example, although described as being arranged in an umbilical cord 2006, the optical fibers 4006 and 4010 can be arranged side-by-side as in a ribbon cable. Furthermore, although described as having an increased number of illuminator optical fibers 4006 in each group 4000, 4002, and 4004 as the radius r increases, where each illuminator optical fiber sources, at its end, electromagnetic energy having an approximately same intensity, the probe head 2010 can include a same or similar number of illuminator optical fibers in each group and the system 2000 (FIG. 2) can be configured to provide a higher intensity of electromagnetic energy to each fiber in a group as the radius r increases such that the total intensity of electromagnetic energy from each radius r is approximately the same at the collecting end of the at least one collector optical fiber 4010. Or, to achieve the same result, the probe head 2010 still can include a same or similar number of illuminator optical fibers in each group and the system 2000 (FIG. 2) can be configured to provide electromagnetic energy to each fiber in a group for a longer time as the radius r increases such that the total level of electromagnetic energy (where “level” effectively is an integral of intensity) from each radius r is approximately the same at the collecting end of the at least one collector optical fiber 4010. Moreover, instead of, or in addition to, the adhesive surface 4012, the probe head 2010 can include another structure, such as a strap, to secure the probe head to a body part such as the thenar eminence of a hand. In addition, instead of disposing the electromagnetic-energy generator 2022 in the base 2004, the generator, including the LED array 2044, driver circuit 2046, and the temperature controller 2024, can be disposed partially or fully in the probe head 2010 or in one other portions of the probe 2002; such an embodiment would eliminate the need for running the illuminator optical fibers 4006 to the base 2004. Furthermore, the authenticator 4018 can be disposed in the probe connector 2008 (FIG. 2) instead of in the probe 2010. Moreover, although the LEDs of the array 2044 (FIG. 2) are described as each radiating approximately the same spectrum and intensity of electromagnetic energy, the LEDs can be configured to radiate different spectra, different intensities, or both different spectra and different intensities. For example, the LEDs configured to drive the illuminator optical fibers 4006 in the group 4004 (fiber ends at radial distance r₃) can have approximately the same spectra, which are different from the spectra of the LEDs configured to drive the illuminator optical fibers in one or both of the groups 4002 and 4000. In addition, although described as being configured for securing to a thenar eminence of a hand, the probe head 2010 can be configured for securing to any other suitable body part, for example, to a body part including skeletal muscle and where the thickness of the skin and fat layers over the muscle is no more than approximately 3 mm; examples of such a body part include the bottom of a foot, back of a hand, neck (sternocleidomastoid muscle), chest (pectoralis major), and the limbs of babies and younger children. Moreover, in an embodiment of the probe 2002 including multiple illuminator optical fibers 4006 and multiple collector optical fibers 4010, instead of time multiplexing the activation of the sets 4000, 4002, and 4004 of the illuminator optical fibers 4006, the base 2004 can include a respective spectrometer 2026 for each of the collector fibers, or can include a series of dichroic filters to carve out “slices” of the spectra and then effectively recombine these slices with the computing circuit 2048, so that all sets of illuminator optical fibers can be energized simultaneously; that is, the multiplexing and demultiplexing is in the wavelength domain instead of the time domain; for example, in an embodiment with three collector optical fibers 4010, the base 2004 can include three spectrometers 2026, one spectrometer per collector optical fiber. Alternatively, the spectrometer 2026 can include a two-dimensional (instead of a linear) sensor, and the probe receptacle 2020 and the probe connector 2008 can be configured to direct the collected electromagnetic energy from each collector optical fiber 4010 onto a respective region of the two-dimensional sensor. In yet another alternative, the spectrometer 2026 includes an input optical multiplexer, and the control circuit 2028 is configured to control the multiplexer to direct the collected electromagnetic energy output from each of multiple collector optical fibers 4010 onto the spectrometer sensor at a respective time, so that at any given time the control circuit “knows” which collector optical fiber is providing the output being acted upon by the spectrometer. As stated elsewhere in this document, in an embodiment of the system 2000 including multiple collector optical fibers 4010, the system can include as few as one illuminator optical fiber 2006.

Furthermore, the authentication circuit 4018 could employ conventional rolling codes to deter hacking of authentication data from the memory 4020 and to deter a hacker taking control of the transmitter 4024 to transmit a counterfeit authentication code to the control circuit 2028 (FIG. 2). And the authentication memory 4020 can store particular data related to the probe, such as measurements taken, and test results generated, during production of the probe 2002; after the probe is connected to the base 2004, as part of a calibration procedure, the control circuit 2028 can compare some or all of the data to equivalent data generated by the control circuit to determine whether the probe is malfunctioning or damaged and otherwise properly calibrated. In addition, embodiments described in conjunction with FIGS. 1-3 and 4B-17 may be applicable to the probe head 2010 of FIG. 4A.

FIG. 4B is a perspective view of an attachment member 4100 configured for use with the probe head 2010 of the probe 2002 of FIGS. 2 and 3B and for attaching to a thenar eminence 4102 of a subject's hand 4104, according to another embodiment. The attachment member 4100 is also configured for use with an embodiment of the probe head 2010 of FIG. 4A in which the probe head lacks the adhesive surface 4012.

The attachment member 4100 includes a flexible adhesive mesh 4106 and a probe-head receptacle 4108 attached to the adhesive mesh by adhesive, stitching, or other suitable attachment technique.

The flexible adhesive mesh 4106 includes, on its attachment side facing the hand 4104, suitable adhesive, which can be conventional, and a protective film (not shown in FIG. 4B) that a user removes before affixing the attachment portion 4100 to the thenar eminence 4102. The mesh may be made of plastic, cloth, or a combination of plastic and cloth.

The mesh 4106 also includes, beneath the probe-head receptacle 4108, at least one opening that allows the electromagnetic energy generated by the electromagnetic-energy generator 2022 (FIG. 2) to propagate unimpeded from the output ends of the illuminator optical fibers 4006 (FIG. 4A) to the skin, muscle, and other tissues that form the thenar eminence 4102, and that allows the portion of the illumination electromagnetic energy redirected by the tissue to propagate unimpeded to the one or more collector optical fibers 4010 (FIG. 4A).

The probe-head receptacle 4108 can be made from any suitable material such as plastic, and is constructed, or otherwise configured, for receiving the probe head 2010 (e.g., FIGS. 3B and 4A (in an embodiment in which the adhesive surface 4012 is omitted), and 4C). For example, the receptacle 4108 and the probe head 2010 can be configured for one to “snap” the probe head into the receptacle 4108, or the outside of the probe head and the inside of the receptacle can be threaded so that one can twist, or screw, less than or more than a full turn, the probe head into the receptacle. Furthermore, the receptacle 4108 and the probe head 2010 can be constructed, or otherwise configured, so that one can attach the probe head to the receptacle before or after the attachment member 4100 is secured to the subject's thenar eminence 4102 (or other body part). In addition, the receptacle 4108 has, through its bottom 4110, one or more openings configured to allow electromagnetic energy emanating from the illuminator optical fibers 4006 (FIG. 4A) and electromagnetic energy redirected by the subject's tissue toward the one or more collector optical fibers 4010 (FIG. 4A) to pass through the bottom unimpeded.

Still referring to FIG. 4B, alternate embodiments of the attachment member 4100 are contemplated. For example, although described as being round, the probe-head receptacle 4108 can have any suitable shape, and can have any suitable color, such as a black having low reflectance in the wavelength range of the electromagnetic energy generated by the electromagnetic energy generator 2022 of FIG. 2. Furthermore, the adhesive mesh 4106 can have any suitable shape and color. In addition, although the probe head 2010 is described as fitting inside of the receptacle 4108, the receptacle can fit inside of the probe head. Moreover, embodiments described in conjunction with FIGS. 1-4A and 4C-18 may be applicable to the probe-head receptacle 4108 of FIG. 4B.

FIG. 4C is a perspective view of a probe head 2010 of, e.g., FIGS. 2, 3B, and 4A inserted into, or otherwise secured, to the receptacle 4108 of the attachment member 4100 of FIG. 4B, according to an embodiment. The probe head 2010 has a two-piece (e.g., clamshell) construction, with the two pieces (top and bottom from the perspective of FIG. 4C) held together by machine screws 4200.

The probe head 2010 can be packaged attached to the attachment member 4100 to protect a face (the side facing a subject's body and disposed inside of the receptacle 4108 while the probe head is in use) of the probe head while the probe head is packaged and in storage awaiting use, or a protective film, similar to the film 4014 of FIG. 4A, can be attached (e.g., by an adhesive) to the face of the probe head as described in conjunction with FIG. 4A. In the former case, a user can affix the attachment member 4100 to a subject's body without removing the probe head 2010 from the receptacle 4108, or can remove the probe head from the receptacle, affix the attachment member to the subject's body, and reinsert the probe head into, or otherwise reattach the probe head to, the receptacle. If, for some reason, the probe head 2010 needs to be disengaged with the receptacle 4108, the adhesive and receptacle 4108 can remain secured on the subject's body and re-engaged with the probe head 2010 to resume testing of the subject's body.

Still referring to FIG. 4C, alternate embodiments of the attachment member 4100 and probe head 2010 are contemplated. For example, embodiments described in conjunction with FIGS. 1-4B and 4D-18 may be applicable to the attachment member 4100 and the probe-head 2010 of FIG. 4C.

FIG. 4D is a perspective view of the probe head 2010 of FIGS. 2, 3B, 4A, and 4C attached to a calibration member, or calibrator, 4300, according to an embodiment. The probe head 2010 can be configured for attaching to the calibration member 4300 in a manner similar to the way in which it is configured for attaching to the attachment member 4100 of FIG. 4B, and the calibrator 4300 has a calibration surface 4302 (the surface that faces the probe head 2010 while the probe head is attached to the calibrator) that is white and that is otherwise similar to the calibration surface 4016 of the protective film 4014 (FIG. 4A). One can use the calibrator 4300 to calibrate the system 2000 if the protective film 4014 is intentionally or inadvertently missing from the probe head 2010 or if the calibration surface 4016 of the protective film is damaged.

Still referring to FIG. 4D, alternate embodiments of the probe head 2010 and calibrator 4300 are contemplated. For example, embodiments described in conjunction with FIGS. 1-4C and 5-18 may be applicable to the probe head 2010 and the calibrator 4300 of FIG. 4D.

FIG. 5 is a flow diagram 5000 of a method for using, and a method of operating, the cell-oxygenation-level determiner-and-monitor system 2000 of FIG. 2, according to an embodiment. The method is described according to an embodiment in which the probe 2002 of FIG. 4A is used, it being understood that the method can be similar if another version of the probe is used.

Referring to FIGS. 2-5, at a step 5002, a user, such as an EMS or CCS (e.g., nurse, doctor, technician) connects the probe 2002 to the base 2004 by inserting the probe connector 2008 into the probe receptacle 2020. In response to the user inserting the probe connector 2008 far enough into the receptacle 2020 to “trip” the sensor 2040, the control circuit 2028, in response to a sense signal from the sensor, activates the probe-connector clamping mechanism (FIGS. 13-14) to engage the probe connector and to hold the probe connector within the probe receptacle in stable optical alignment with the LED array 2044 (see FIG. 6).

Next, at a step 5004, the control circuit 2028 determines whether the probe 2002 is authorized for use with the base 2004. For example, the control circuit 2028, via the network connector 2036 operating in a wireless mode, sends a request for an authentication value to the wireless transmitter 4024 (FIG. 4A) of the probe head 2010 (the transmitter can include, or can be part of, a microprocessor, microcontroller, or other circuitry that processes the request), and, in response to the request, the transmitter retrieves the stored authentication value from the memory 4020 (FIG. 4A), codes the authentication value, and sends the coded authentication value to the control circuit via the network connector. The control circuit 2028 decodes the authentication value and determines whether the authentication is valid. For example, the control circuit 2028 compares the authentication value to a list of one or more valid authentication values stored in the memory 2030 and enables the base 2004 for use with the probe 2002 if the received authentication value matches one of the stored authentication values. Alternatively, the control circuit 2028 processes the received authentication value according to an authentication algorithm and compares the result to a list of one or more valid results stored in the memory 2030, and enables the base 2004 for use with the probe 2002 if the obtained result matches one of the stored results. If the control circuit 2028 determines that the received authentication value is valid, then the control circuit proceeds to a step 5006. But if the control circuit 2028 determines that the authentication value or algorithm result is invalid, then the control circuit proceeds to a step 5008, at which the control circuit disables the base 2004 from operating while the unauthenticated probe 2002 is installed.

At the step 5006, the base 2004 performs spectral calibration. Referring to FIGS. 2 and 4A, the control circuit 2028 first generates, on the display 2032, a message notifying a user of the cell-oxygenation-level determiner-and-monitor system 2000 that a calibration is taking place and instructing the user not to remove the protective strip 4014 from the adhesive surface 4012 of the probe head 2010. The control circuit 2028 next causes the electromagnetic-energy generator 2022 to “flash” the LEDs in the array 2044 over one or more calibration times. In more detail, the control circuit 2028 activates the LEDs in the array 2044 corresponding to the group 4000 of illuminator optical fibers 4006 for a first calibration time in an approximate range of 0.1 ms-1 ms, for example, approximately 0.4 ms, and deactivates the remaining LEDs in the array. The electromagnetic energy from the active LEDs propagates along the illuminator optical fibers 4006 in the group 4000 and exits the output end of each of these fibers. The, e.g., white, calibration surface 4016 of the protective strip 4014 redirects a portion of the incident electromagnetic energy into the input end of the collector optical fiber 4010, and the spectrometer 2026 spatially separates the wavelengths of the redirected electromagnetic energy into ranges as described in conjunction with FIG. 2. For each range, the spectrometer 2026 generates a respective calibration value corresponding to the combined intensity of the wavelengths in the range. The control circuit 2028 then stores, in the memory 2030, the one or more calibration values corresponding to the illuminator-optical-fiber group 4000. Next, the control circuit 2028 activates the LEDs in the array 2044 corresponding to the group 4002 of illuminator optical fibers 4006 for a second calibration time in an approximate range of 1 ms-10 ms, for example, approximately 4 ms, and deactivates the remaining LEDs in the array. The electromagnetic energy from the active LEDs propagates along the illuminator optical fibers 4006 in the group 4002 and exits the output end of each of these fibers. The calibration surface 4016 of the protective strip 4014 redirects a portion of the incident electromagnetic energy into the input end of the collector optical fiber 4010, and the spectrometer 2026 spatially separates the wavelengths of the redirected electromagnetic energy into ranges, and, for each range, generates a respective calibration value corresponding to the combined intensity of the wavelengths in the range. The control circuit 2028 then stores, in the memory 2030, the one or more calibration values corresponding to the illuminator-optical-fiber group 4002. Then, the control circuit 2028 activates the LEDs in the array 2044 corresponding to the group 4004 of illuminator optical fibers 4006 for a third calibration time in an approximate range of 1 ms-10 ms, for example, approximately 8 ms, and deactivates the remaining LEDs in the array. The electromagnetic energy from the active LEDs propagates along the illuminator optical fibers 4006 in the group 4004 and exits the output end of each these fibers. The calibration surface 4016 of the protective strip 4014 redirects a portion of the incident electromagnetic energy into the input end of the collector optical fiber 4010, and the spectrometer 2026 spatially separates the wavelengths of the redirected electromagnetic energy into ranges. For each range, the spectrometer 2026 generates a respective calibration value corresponding to the combined intensity of the wavelengths in the range. The control circuit 2028 then stores, in the memory 2030, the one or more calibration values corresponding to the illuminator-optical-fiber group 4004. The control circuit 2028 performs a similar calibration for each remaining group (if any) of illuminator optical fibers 4006. Next, the control circuit 2028 generates, via a sound generator (not shown in FIG. 2) or on the display 2032, a notification that the calibration procedure is complete and that the user can remove the protective strip 4014 from the face of the probe head 2010. Alternatively, if the control circuit 2028 can complete the calibration procedure so quickly that, after installing the probe 2002, no human would be fast enough to remove the protective strip 4014 before the calibration procedure is finished, then the control circuit 2028 can omit generating any calibration-related messages via a sound generator or on the display 2032. In an alternative embodiment in which a separate calibrator (e.g., the calibrator 4300 of FIG. 4D) is used instead of the calibration surface 4016 of the protective strip 4014, the step 5006 is altered to include removing the protective strip 4014 from the probe head 2010 and installing the external calibrator onto the probe head before the calibration procedure is performed.

Then, at a step 5010, the control circuit 2028 determines whether each of the calibration values is within a range of suitable values. For example, the control circuit 2028 compares each calibration value to a suitable calibration-value threshold or range stored in the memory 2030 for the spectral range with which the calibration value corresponds.

If one or more of the calibration values are out of range, then there may be a problem with one or more components of the system 2000, such as a malfunction of, or damage to, one or more of the LEDs of the array 2044, the driver circuit 2046, the temperature controller 2024, the spectrometer 2026, or the probe 2002. And such a problem may require that the system 2000 be serviced or repaired before being used, or that a clinician replace a damaged, or otherwise malfunctioning, probe 2002 with another probe. For example, a calibration value for a particular wavelength range may be out of specification if the calibration value is less than a threshold value corresponding to 0.5 lumens.

Consequently, if, at the step 5010, the control circuit 2028 determines that at least one of the calibration values is outside of a respective suitable range of values, then the method proceeds to a step 5012, at which the control circuit 2028 disables the base 2004 from measuring cell-oxygen-saturation levels and generates, on the display 2032, an error message that indicates, for example, that the system 2000 needs service before it can be used. Alternatively, the control circuit 2028 does not disable the base 2004 but generates, on the display 2032, a message indicating that the system 2000 needs to be serviced and may yield inaccurate readings of cell-oxygenation level until it is serviced.

But if, at the step 5010, the control circuit 2028 determines that each of the calibration values has an acceptable relationship to a respective threshold or is within a respective suitable range of values, then the method proceeds to a step 5014.

At the step 5014, the user removes the protective strip 4014 from the probe head 2010, places the adhesive surface 4012 over a subject's hand in the region of the thenar eminence, and presses the probe head against the hand to affix the probe head to the hand. If the probe head 2010 includes a strap or other attachment structure in addition to, or instead of, the adhesive surface 4012, then the user secures the probe head over the thenar eminence of the subject's hand accordingly.

Next, at a step 5016, the control circuit 2028 causes the probe head 2010 to illuminate the subject's tissue with electromagnetic energy in an approximate wavelength range of 500 nm-850 nm over a cell-oxygen-saturation-level measurement time. The control circuit 2028 first activates the LED in the array 2044 corresponding to the group 4000 of one illuminator optical fiber 4006 for a first illumination time in an approximate range of 0.1 ms-1.0 ms, for example, approximately 0.4 ms, and deactivates the remaining LEDs in the array. The electromagnetic energy from the active LED propagates along the illuminator optical fiber 4006 in the group 4000 and exits the output end of this fiber. The exiting electromagnetic energy diffuses into the subject's tissue, the cells that form the subject's tissue (e.g., skeletal-muscle cells) absorb a portion of the diffused electromagnetic energy, the subject's tissue redirects another portion of the diffused electromagnetic energy into the input end of the collector optical fiber 4010, and the redirected portion (also called the “collected portion”) of the electromagnetic energy propagates through the collector optical fiber 4010 to the spectrometer 2026. The time that it takes for the electromagnetic energy emanating from the output end of the one illuminator optical fiber 4006 in the group 4000 to diffuse into the subject's tissue, for the cells composing the tissue to absorb a portion of the diffused electromagnetic energy, for the tissue to redirect another portion of the diffused electromagnetic energy to the input end of the collector optical fiber 4010, and for the collected electromagnetic energy to propagate along the collector optical fiber 4010 to the spectrometer 2026, is significantly less than, for example, no more than approximately one tenth of, the first illumination time.

The control circuit 2028 next activates the LEDs in the array 2044 corresponding to the group 4002 of two illuminator optical fibers 4006 for a second illumination time in an approximate range of 1.0 ms-10.0 ms, for example, approximately 4.0 ms, and deactivates the remaining LEDs in the array. The electromagnetic energy from the active LEDs propagates along the two illuminator optical fibers 4006 in the group 4002 and exits the output ends of these fibers. The exiting electromagnetic energy diffuses into the subject's tissue, the cells that form the subject's tissue (e.g., skeletal-muscle cells) absorb a portion of the diffused electromagnetic energy, the subject's tissue redirects another portion of the diffused electromagnetic energy into the input end of the collector optical fiber 4010, and the redirected portion of the electromagnetic energy propagates through the collector optical fiber 4010 to the spectrometer 2026. The time that it takes for the electromagnetic energy emanating from the output ends of the illuminator optical fibers 4006 in the group 4002 to diffuse into the subject's tissue, for the cells composing the tissue to absorb a portion of the diffused electromagnetic energy, for the tissue to redirect another portion of the diffused electromagnetic energy to the input end of the collector fiber 4010, and for the collected electromagnetic energy to propagate along the collector optical fiber 4010 to the spectrometer 2026, is significantly less than, for example, no more than approximately one tenth of, the second illumination time.

Still at the step 5016, the control circuit 2028 then activates the LEDs in the array 2044 corresponding to the group 4004 of four illuminator optical fibers 4006 for a third illumination time in an approximate range of 1.0 ms-10.0 ms, for example, approximately 8.0 ms, and deactivates the remaining LEDs in the array. The electromagnetic energy from the active LEDs propagates along the four illuminator optical fibers 4006 in the group 4004 and exits the output ends of these fibers. The exiting electromagnetic energy diffuses into the subject's tissue, the cells that form the subject's tissue (e.g., skeletal-muscle cells) absorb a portion of the diffused electromagnetic energy, the subject's tissue redirects another portion of the diffused electromagnetic energy into the input end of the collector optical fiber 4010, and the redirected portion of the electromagnetic energy propagates through the collector optical fiber 4010 to the spectrometer 2026. The time that it takes for the electromagnetic energy emanating from the output ends of the illuminator optical fibers 4006 in the group 4004 to diffuse into the subject's tissue, for the cells composing the tissue to absorb a portion of the diffused electromagnetic energy, for the tissue to redirect another portion of the diffused electromagnetic energy to the input end of the collector optical fiber 4010, and for the collected electromagnetic energy to propagate along the collector optical fiber 4010 to the spectrometer 2026, is significantly less than, for example, no more than approximately one tenth of, the third illumination time.

Then, at a step 5018, which is concurrent with the step 5016, the spectrometer 2026 decomposes, into wavelength ranges, the redirected electromagnetic energy, which is collected, and which is provided to the spectrometer input, by the group 4008 of one collector optical fiber 4010. For example, in an embodiment in which the wavelengths in the spectra of the redirected electromagnetic energy span an approximate range of 501 nm-850 nm, the spectrometer 2026 sensor has a wavelength resolution of approximately 0.31 nm per pixel such that the spectrometer decomposes the collected electromagnetic energy into approximately eleven hundred twenty six (1126) wavelength bins, or ranges (one bin/range per pixel), that are each approximately 0.31 nm wide. But for purposes of illustration, it is assumed that the spectrometer 2026 decomposes the collected electromagnetic energy into the following thirty five approximate wavelength ranges: 501 nm-510 nm, 511 nm-520 nm, 521 nm-530 nm, . . . , 831 nm-840 nm, and 841 nm-850 nm. It is understood, however, that the spectrometer 2026 can be configured to decompose the collected electromagnetic energy into any suitable number of wavelength bins, or ranges, over any suitable wavelength span, and that the following description would still apply.

And, for each wavelength range, the spectrometer 2026 generates one or more values respectively related to one or more characteristics of the wavelengths in the wavelength range. For example, the spectrometer 2026 generates a value related to the combined intensity (e.g., magnitude, amplitude, power) of the wavelengths in the wavelength range. Further in example, the spectrometer 2026 generates, as a representative of each value, a respective analog electrical signal, and converts the analog signal into a corresponding digital signal. Still further in example, the spectrometer 2026 generates a number of digital samples of each characteristic for each wavelength range, and the control circuit 2028 mathematically combines (e.g., averages) these samples to generate a respective value for each analyzed spectral characteristic. Yet further in example, if the spectrometer 2026 generates digital values 5.0, 6.0, 8.0, 4.0 over four samples of intensity for the wavelength range 511 nm-520 nm, then the control circuit 2028 generates, and stores in the memory 2030, a value (5.0+6.0+7.0+4.0)/2=11.0 of intensity for this wavelength range.

For purposes of clarity, in the following description the spectrometer 2026 generates, for each wavelength range, only values only for intensity, it being understood, however, that the spectrometer can generate, for each wavelength range, values for other wavelength-range characteristics (e.g., phase, polarity) instead of, or in addition to, intensity.

Still at the step 5018, during the first illumination time, for each wavelength range of the collected electromagnetic energy, the spectrometer 2026 generates a respective value related to intensity as described above, and the control circuit 2028 stores the respective value in the memory 2030 such that the value is associated with the wavelength range and the radius r₁ of the group 4000 of one illuminator optical fiber 4006.

During the second illumination time, for each wavelength range of the collected electromagnetic energy, the spectrometer 2026 generates a respective value related to intensity, and the control circuit 2028 stores the respective value in the memory 2030 such that the value is associated with the wavelength range and the radius r₂ of the group 4002 of two illuminator optical fibers 4006.

During the third illumination time, for each wavelength range of the collected electromagnetic energy, the spectrometer 2026 generates a respective value related to intensity, and the control circuit 2028 stores the respective value in the memory 2030 such that the value is associated with the wavelength range and the radius r₃ of the group 4004 of four illuminator optical fibers 4006.

And during each subsequent illumination time, if any, for each wavelength range of the collected electromagnetic energy, the spectrometer 2026 generates a respective value related to intensity, and the control circuit 2028 stores the value in the memory 2030 such that the value is associated with the wavelength range and the radius rₙ of the respective group of one or more illuminator optical fibers 4006.

As described above, the number of illuminator optical fibers 4006 in a group increases with increasing radius r such that the level (e.g., the number of photons) of electromagnetic energy that the one or more (here one) collector optical fibers 4010 collect during each of the first, second, third, and any subsequent, illumination periods is approximately equal. Alternatively, the levels energy collected during the illumination periods need not be approximately equal to one another as long as the respective level of energy collected during each illumination period has at least a minimum-threshold energy level, which minimum threshold can be defined in terms of the signal-to-noise ratio (S/N) of the energy received by the one or more collector optical fibers 4010.

Next, at a step 5020, the computing circuit 2048 of the control circuit 2028 determines, in response to the values stored in the memory 2030 during the first, second, third, and any subsequent, illumination times, a level of cell-oxygenation in the subject's tissue that the probe 2002 illuminated with electromagnetic energy. For example, the determined level of cell-oxygenation may be, effectively, an average, or other mathematical combination, of the cell-oxygenation levels of each illuminated cell in the tissue. And, unless it “knows” otherwise, the control circuit 2028 “assumes” that the cell-oxygenation level determined for the illuminated tissue is approximately the same as the cell-oxygenation level in similar tissue (e.g., skeletal-muscle tissue) in other parts of the subject's body. For example, while an automatic sphygmomanometer (i.e., blood-pressure machine) signals the control circuit 2028 that a blood-pressure cuff, which is on the arm with the hand to which the probe head 2010 is attached, is inflated, the control circuit can be configured to ignore cell-oxygenation-level readings computed by the computing circuit 2048 because the readings may not accurately reflect cell oxygenation in other parts of the subject's body due to an ischemia that the inflated cuff induces in the probed hand. Alternatively, if the time during which the cuff is inflated is shorter than a threshold, and, therefore, is not long enough to cause inaccurate cell-oxygenation readings, then the control circuit 2028 can be configured to accept the readings as accurate, at least until the cuff-inflation time increases to or above the threshold. Furthermore, as part of determining the cell-oxygenation level, the computing circuit 2048 may calculate oxymyoglobin% (cell-oxygen-saturation level)=[oxymyoglobin]/([oxymyoglobin]+[deoxymyoglobin]) and oxyhemoglobin% (blood-oxygen-saturation level)=[oxyhemoglobin]/([oxyhemoglobin]+[deoxyhemoglobin]).

First, the computing circuit 2048 normalizes each of the values stored in the memory 2030 during the first, second, third, and any subsequent, illumination times. For each stored value being normalized, the computing circuit 2048 first divides the corresponding calibration value, previously determined and stored in the memory 2030 at the step 5006, by the stored value. For example, if “550” is the decimal representation of the digital value stored for the wavelength range 501 nm-510 nm at radial distance r₁ during the first illumination time and “500” is the decimal representation of the corresponding digital calibration value stored for the same wavelength range and same radial distance r₁ during the calibration procedure, then the computing circuit 2048 generates the quotient 500/550=10/11=0.91. Next, the computing circuit 2048 generates the normalized version of the value “550” for the wavelength range 501 nm-510 nm at the radial distance r₁ for the first illumination time equal to log₁₀(0.91)=−0.41. Alternatively, the computing circuit 2048 generates the normalized version equal to |log₁₀(0.91)|=0.41. The computing circuit 2048 normalizes each of the other values stored in the memory 2030 during the first, second, third, and any subsequent, illumination times in a similar manner. Normalizing the values can reduce the magnitude of, or altogether eliminate, errors caused by shifts in the spectra generated the electromagnetic-energy generator 2022 due to, for example, aging of the LEDs in the array 2044, shifts in temperature of, or in the supply voltage provided to, the generator from one subject to the next, and differences in electromagnetic-energy illuminating and collecting from probe 2002 to probe.

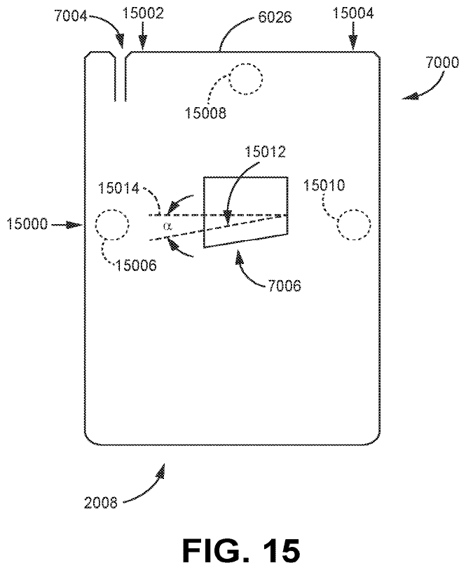

Then, the computing circuit 2048 processes the values stored in the memory 2030 during the first, second, third, and any subsequent, illumination times by applying the normalized versions of these values as inputs to a mathematical algorithm that the computing circuit executes or otherwise performs. Examples of suitable mathematical algorithms include learning algorithms (e.g., machine-learning and statistical-learning algorithms) such as locally weighted regression (LWR) models (see, for example, col. 9, lines 25-32 of U.S. Pat. No. 10,463,286), support vector machines, decision-tree methods (e.g., random forest, XGBoost), neural networks (NN) (e.g., feed forward, convolutional (CNN), recurrent, generative adversarial), and recommender systems. For each input value, the respective information available to the designer of the algorithm (and for input to the algorithm itself) includes the distance rₙ between the corresponding illumination optical fiber(s) 4006 and the one or more collector optical fibers 4010, the durations of the first, second, third, and subsequent illumination times, and the one or more wavelengths in the wavelength range with which the input value is associated. Consequently, based on this available information, the algorithm designer constructs the algorithm with the ability to be trained (training the algorithm is described below in conjunction with FIGS. 15-17) to yield accurate values of cell-oxygenation.

Still at step 5020, the computing circuit 2048 accurately determines the level of oxygenation of the cells in the tissue (e.g., skeletal-muscle tissue) illuminated by the generated electromagnetic energy per step 5016. As described above, the determined level of cell-oxygenation may be, effectively, an average, or other mathematical combination, of the respective cell-oxygen-saturation levels of the cells that form the tissue.

Next, at a step 5022, the control circuit 2028 generates, on the display 2032, the determined level of cell-oxygenation, for example, “95.6%.” The control circuit 2028 also generates, on the display 2032, other information such as an indication as to whether the displayed level of cell-oxygenation is within a normal range (e.g., 90%-100%), is within a cautionary range (e.g., 70%-89.9%), or is within an emergency range (e.g., <70%). The control circuit 2028 also generates an audio, visual, or both and audio and visual alarm, e.g., to alert a nurse, if the subject's cell-oxygen-saturation level is outside of the normal range.

Then, at a step 5024, the control circuit 2028 determines whether it is to stop determining and monitoring the cell-oxygen-saturation level of the subject. For example, a user can enter a “stop” command to the control circuit 2028 via the interface 2034. Or, in response to one activating the probe-connector release 2042, or in response to the sensor 2040 generating a signal indicative of no probe connector 2008 being within the receptacle 2020, the control circuit 2028 can cease determining and monitoring the cell-oxygen-saturation level, at least until another probe 2002 is attached to, and is authorized by, the control circuit.

If the control circuit 2028 determines that the system 2000 is to cease determining and monitoring the cell-oxygen-saturation level of the subject, then it ends the method represented by the flow chart 5000.

Otherwise, the control circuit 2028 proceeds to a step 5026.

At the step 5026, the control circuit 2028 implements a delay having a duration that can ranges from a suitable minimum duration, which can be as low as 0.0 seconds, to a suitable maximum delay, such as 60 seconds or more. The control circuit 2028 can include a counter or other clock circuit for measuring the delay, or can execute instructions of a clock, or of another time-measuring software application, to implement the delay.

After the delay has elapsed, the control circuit 2028 returns to the step 5016, and repeats the steps 5016, 5018, 5020, 5022, 5024, and 5026 until, at the step 5024, the control circuit 2028 determines that the system 2000 is to cease determining and monitoring the cell-oxygenation level of the subject.

Still referring to FIGS. 2, 3, 4, and 5, the system 2000 and method represented by the flow chart 5000 can provide one or more advantages over previously proposed systems for determining a cell-oxygenation level. For example, collecting and analyzing wavelengths of electromagnetic energy over a wide spectrum (e.g., approximately 400 nm to 900 nm or 500 nm to 800 nm) can yield more accurate readings of cell-oxygenation (e.g., myoglobin-oxygen saturation) than collecting and analyzing only a few (e.g., one to five) wavelengths. Furthermore, collecting analyzing wavelengths in two or more spectral regions in a wavelength range of approximately 400 nm-900 nm or 500 nm-800 nm of the electromagnetic spectrum can yield more accurate readings of cell-oxygen-saturation than collecting and analyzing wavelengths in only one of these spectral regions. In addition, collecting tissue-redirected electromagnetic energy that has diffused into the tissue over multiple radial (e.g., lateral) distances can yield more accurate readings of cell oxygenation than collecting tissue-redirected electromagnetic energy that has diffused into the tissue over only one radial distance.

Still referring to FIGS. 2, 3, 4A-4D, and 5, alternate embodiments of the method of using and operating represented by the flow diagram 5000 are contemplated. For example, instead of storing an authentication value in a memory 4020, the probe head 2010 can include an authentication barcode that a user scans and enters into the base 2004 directly (the base 2004 can incorporate a barcode scanner) or via a separate barcode scanner and the interface 2034, or a user goes online, presents a serial or other number disposed on, or associated with, the probe 2002, and obtains an authentication code for entering into the base via the interface 2034. And instead of disabling the system 2000 in response to determining that a probe 2002 is unauthorized, the control circuit 2028 allows the system to operate but sends, via the internet, information to the system 2000 manufacturer that informs the manufacturer that an unauthorized probe 2002 was used so that the manufacturer can take appropriate action. Furthermore, the order in which the circuit 2028 activates the groups of the illuminator optical fibers 4006 during the calibration and cell-oxygen-saturation-level determining-and-monitoring procedure can be other than closest (smallest radial distance r) to farthest (largest radial distance), and the calibration procedure may be different than described. In addition, instead of including an increasing number of illuminator fibers 4006 in each group of one or more illuminator fibers as the radius r increases, the number of illuminator fibers in each group can be the same or similar and the control circuit 2028 can control the electromagnetic-energy generator 2022 to generate higher intensities of electromagnetic energy with increasing radius so that the level of electromagnetic energy collected by the one or more collector optical fibers 4010 for each group of one or more illuminator optical fibers 4006 is approximately the same or has at least a minimum-threshold S/N. regardless of the radial distance between the ends of the illuminator optical fibers and the ends of the one or more collector optical fibers. Moreover, instead of activating the LEDs corresponding to each group of illuminator optical fibers 4006 sequentially, the control circuit 2028 can activate the LEDs corresponding to all groups of illuminator optical fibers simultaneously according to an orthogonal-activation paradigm (e.g., modulation of the frequency at which the LEDs are turned “on” and “off”' or of the intensities of the LEDs according to an orthogonal coding scheme) that allows the control circuit 2028 to determine the respective level of collected electromagnetic energy from each radial distance r; an orthogonal coding scheme may allow use of lower intensities of the electromagnetic energy radiated by the LEDs. Furthermore, although described as having uniform widths (e.g., ˜0.31 nm, ˜10 nm), the wavelength ranges into which the spectrometer 2026 spatially separates the collected electromagnetic energy can be such that the width of one wavelength range equals, approximately, the widths of some, or none, of the other wavelength ranges. In addition, the control circuit 2028 can normalize the values determined for each wavelength range in any suitable manner different than the manner described above. Moreover, the computing circuit 2048 can implement any suitable algorithm to determine a level of cell-oxygenation in response to the normalized wavelength-range values. Furthermore, the control circuit 2028 can repeat the steps 5016, 5018, 5020, and 5022 multiple times before changing the value of cell-oxygen-saturation level on the display 2032; for example, the control circuit can generate the displayed value of cell-oxygen-saturation level as the average, or other mathematical combination, of the values of the cell-oxygen-saturation levels determined over update windows that are each approximately four seconds long. In addition, because the spectral information collected by the one or more collector optical fibers 4010 and processed by the computing circuit 2048 is sufficient for the computing circuit also to determine blood-oxygen saturation level of the subject, the control circuit 2028 can generate, on the display 2032, a value for blood-oxygen-saturation level in addition to generating a value for cell-oxygen-saturation level. In addition, because melanin can impede diffusion of electromagnetic energy in tissue (particularly through the skin), the control circuit 2028 can be configured to control the electromagnetic-energy generator 2022 so that the intensity of the generated electromagnetic energy increases as the darkness of a subject's skin increases and decreases as the darkness of a subject's skin decreases. Moreover, although described as including as few as one collector optical fiber 4010 and multiple illuminator optical fibers 4006 spaced respective distances from the collector optical fiber, the probe head 2010 can include as few as one illuminator optical fiber and multiple collector optical fibers spaced respective distances from the illuminator optical fiber (the intensity of illuminating electromagnetic energy would be related (e.g., proportional) to the distance between the illuminator optical fiber and the active one or more collector optical fibers). Furthermore, although described for use in emergency and critical-care medicine, the system 2000 can be used in other applications, such as veterinary (animals), athletic training, to inform a surgeon at what location to amputate a limb (e.g., where the “line” is between oxygenated and deoxygenated tissue), and to monitor, for transplant, organs (e.g., heart) whose cells contain detectable amounts of myoglobin. For example, a medical team can use the system 2000 to determine and to monitor the cell-oxygenation level of a subject undergoing cardiac surgery or another cardiac procedure, and to notify the surgeon, anesthesiologist, or other member of the team if the subject experiences a low level of cell oxygenation at any point of time during the procedure (“low” can be defined as being at or below a set cell-oxygenation-level threshold). And a medical team can use the system 2000 to determine and to monitor the cell-oxygenation level of a subject undergoing surgery or another procedure in which a subject often experiences significant blood loss, so that the surgeon, anesthesiologist, or other member of the team can use the subject's cell-oxygenation level as a factor in determining whether the subject needs a blood transfusion. Examples of such surgeries and other procedures include spinal surgery and liver-transplant surgery. In addition, the control circuit 2028 can generate an audial or visual (e.g., on the display 2032) alert if one or more of the spectral values being generated by the spectrometer 2026 are out of a suitable range or are above or below a threshold, and the alert can prompt a user to check to make sure the probe 2002 is properly connected to the base 2004 and to the subject. Furthermore, embodiments described in conjunction with FIGS. 1-4D and 6-17 may be applicable to the method represented by the flow diagram 5000 of FIG. 5.

FIG. 6 is a top plan view, with portions transparent and other portions broken away, of the array 2044 of LEDs and a spectrometer input assembly 6014 of the probe receptacle 2020 of FIGS. 2-3B, and of the probe connector 2008 of FIGS. 2-3B installed in the probe receptacle, according to an embodiment.

The array 2044 is a stationary linear array of seven LEDs 6000, 6002, 6004, 6006, 6008, 6010, and 6012 arranged in a single row with their centers spaced apart by a separation distance dLED, which is in an approximate range of 1.5 mm-5.0 mm, and with their electromagnetic-energy-radiating sides facing into the probe receptacle 2020. To provide electromagnetic energy to the first group 4000 of one or more illuminator optical fibers 4006 (one fiber in FIG. 6), the electromagnetic-energy generator 2022 (FIG. 2) is configured to activate the LED 6012. To provide electromagnetic energy to the second group 4002 of one or more illuminator optical fibers 4006 (two fibers in FIG. 6), the generator 2022 is configured to activate the LEDs 6008 and 6010. And to provide electromagnetic energy to the third group 4004 of one or more illuminator optical fibers 4006 (four fibers in FIG. 6), the generator 2022 is configured to activate the LEDs 6000, 6002, 6004, and 6006.

The input 6014 to the spectrometer 2026 (FIG. 2) is adjacent to the LED array 2044 and is configured to receive the redirected electromagnetic energy collected by the collector optical fiber 4010 and to provide the received electromagnetic energy to the portion (e.g., an optical grating or prism) of the spectrometer 2026 that is configured to separate, spatially, the received electromagnetic energy into wavelength ranges. The input 6014 can be any suitable structure, such as an optical train or other optical assembly.

The probe connector 2008 includes a housing 6016 including one or more (seven in FIG. 6) illuminating-fiber channels 6018 configured to hold and secure input ends 6020 of one or more illuminator optical fibers 4006 and one or more (one in FIG. 6) collecting-fiber channels 6022 configured to hold and secure the output ends 6024 of one or more collector optical fibers 4010. The housing 6016 can be made from any suitable material such as plastic or rubber.

The probe connector 2008 further includes a rear side 6026 configured to face the LED array 2044 while the probe connector is installed within the probe receptacle 2020, and the illuminator and collector optical fibers 4006 and 4010 are recessed from the front side by approximately a same distance d_{recess} sufficient to protect the end faces 6028 and 6030 of the respective optical fibers 4006 and 4010 from damage. For example, the distance d_{recess} can be in the approximate range of 0.0 mm-5 mm.

While the probe connector 2008 is installed in the probe receptacle 2020, the back end 6026 of the probe connector and the LEDs of the array 2044 are separated by a distance d_{gap}, which is sufficient to protect the probe connector from damage due to heat generated by the LEDs, to protect the LEDs from damage and from substances (e.g., dirt) that are transferred by the probe connector and that may block, diffuse, or redirect away from the respective channels 6018, the electromagnetic energy that the LEDs radiate, and to protect the spectrometer input 6014 from damage and substances (e.g., dirt) that are transferred by the probe connector and that may block, diffuse, or redirect away from the spectrometer input, collected electromagnetic energy emanating from the end 6024 of the collector optical fiber 4010. And in an embodiment in which d_{recess} equals approximately zero, the separation d_{gap} is sufficient to protect the fiber end faces 6028 and 6030 from damages and substances (e.g., dirt) that may block, diffuse, or redirect electromagnetic energy directed toward the illuminator optical fibers 4006 or emanating from the collector optical fiber(s) 4010. For example, the separation distance d_{gap} can be in the approximate range of 0.0 mm-5 mm.

The receptacle 2020 includes a blocking member 6032 configured to prevent “cross talk” of electromagnetic energy between the illuminator optical fibers 4006 and the collector optical fiber 4010 because such crosstalk may cause an error in one or more cell-oxygen-saturation levels that the computing circuit 2048 (FIG. 2) determines (herein “prevent cross talk” means attenuating cross talk with an attenuation factor that is at least approximately 50%). The blocking member 6032 can have any suitable dimensions and can be made from any material, such as an opaque plastic or metal, suitable to block electromagnetic energy at the one or wavelengths radiated by the LEDs 6000-6012 (see also FIG. 11, which is a side view of the blocking member 6032).

Furthermore, one or both of the distances d_{recess} and d_{gap} also can be sufficiently deep and sufficiently shallow, respectively, to prevent LED “crosstalk,” i.e., to prevent light from an LED (e.g., LED 6000) being incident on the face 6028 or 6030 of an optical fiber aligned with another LED (e.g., LED 6002).

In addition, the diameters of the optical fibers 4006 and 4010 can be approximately the same (e.g., approximately 1.0 mm) as the diameters of the radiating portions of the LEDs in the array 2044.

Still referring to FIG. 6, alternate embodiments of the probe connector 2008 and the receptacle 2020 are contemplated. For example, if the channels 6018 and 6022 are enclosed by electromagnetic-energy-blocking material sufficient to prevent “cross talk” between the illuminator and collector optical fibers 4006 and 4010, then the blocking member 6032 may be omitted. Furthermore, the channels 6018 and 6022 may not be openings in a physical structure, but may be the regions of space that the illuminator and collector optical fibers 4006 and 4010 occupy; and the sheathing around the optical fibers may be made from an electromagnetic-energy blocking material sufficient to prevent cross talk between the optical fibers. In addition, embodiments described in conjunction with FIGS. 1-5 and 7-17 may be applicable to the probe connector 2008, the probe receptacle 2020, or both the probe connector and probe receptacle, of FIG. 6.

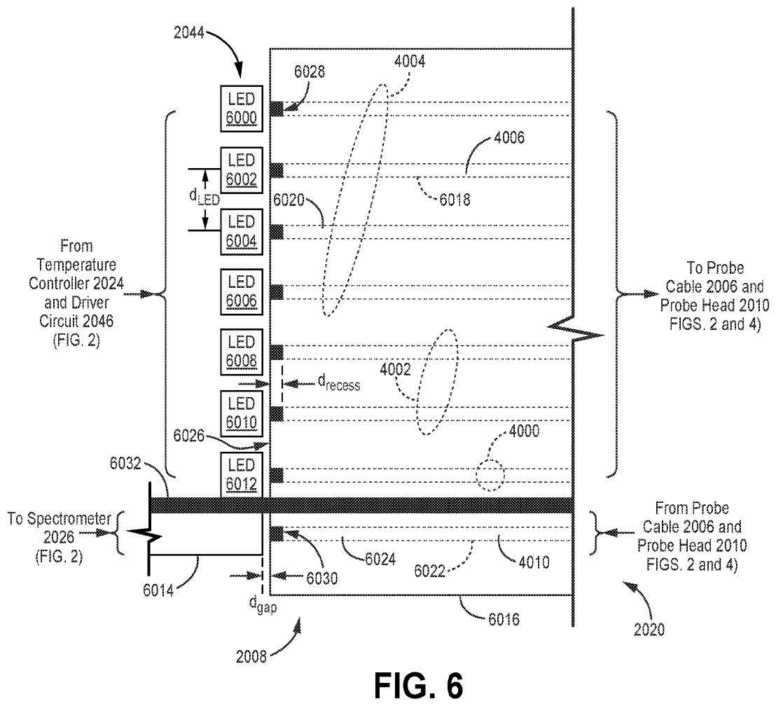

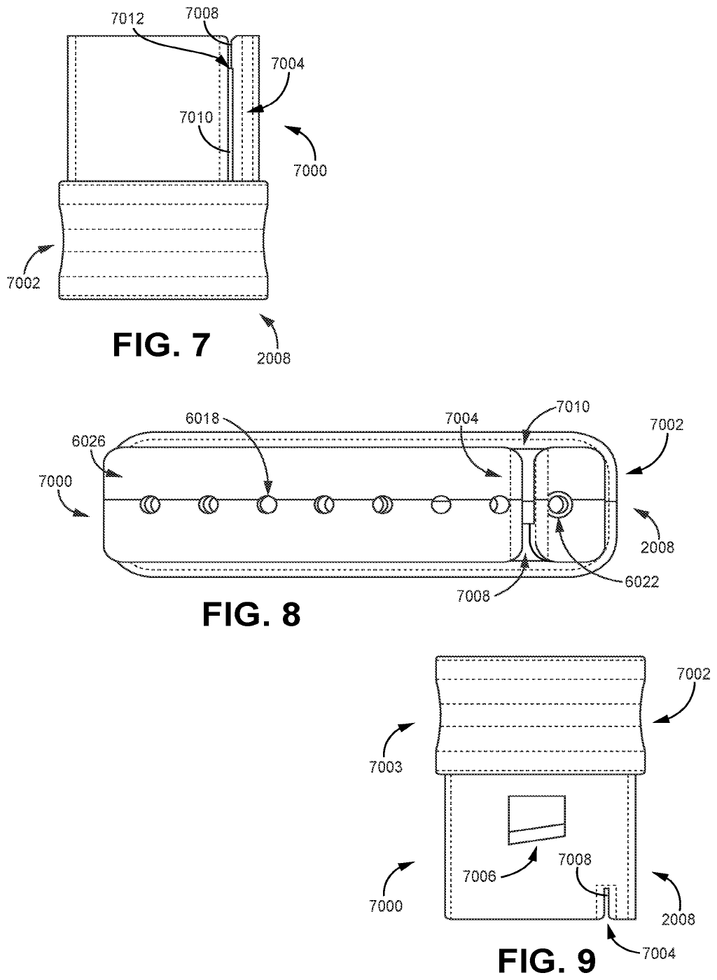

FIG. 7 is a bottom plan view of the probe connector 2008 of FIGS. 2, 3A, 3B, and 6, according to an embodiment.

FIG. 8 is a view of the back end 6026 of the probe connector 2008 of FIGS. 2, 3A, 3B, 6, and 7, according to an embodiment.

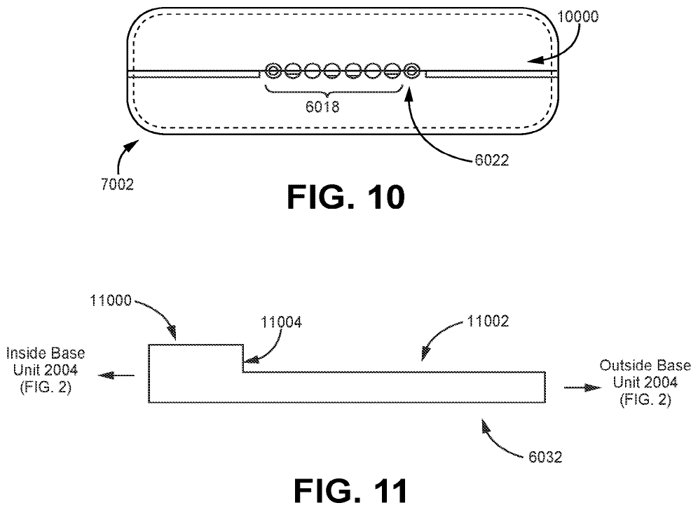

FIG. 9 is a top plan view of the probe connector 2008 of FIGS. 2, 3A, 3B, 6, 7, and 8, according to an embodiment.

FIG. 10 is a view of the front end of the probe connector 2008 of FIGS. 2, 3A, 3B, 6, 7, 8, and 9, according to an embodiment.

Referring to FIGS. 7-10, in addition to the illumination-optical-fiber and the collector-optical-fiber channels 6018 and 6022, the probe connector 2008 includes an insertion back end 7000, a grasping front end 7002, a blocking-member notch 7004, a latch-engagement region 7006 (e.g., a latch-engagement depression or notch), and a front-end face, or surface, 10000.

The insertion back end 7000 includes the backend surface 6026, which is the surface through which the optical-fiber channels 6018 and 6022 open and is constructed for inserting into and engaging the probe receptacle 2020 (FIGS. 2, 3A, 3B, and 6). For example, the back end 7000 can be formed from a rigid material such as a hard plastic.

The grasping front end 7002 is attached to, or integral with, the insertion back end 7000, and is constructed to be grasped by a human hand to facilitate one inserting the insertion back end into, and removing the insertion back end from, the probe receptacle 2020 (FIGS. 2, 3A, 3B, and 6). For example, the grasping front end 7002 includes a concave portion 7003 configured to facilitate gripping by a hand and is formed from a pliable material, such as a vinyl or vinyl-covered foam. The optical-fiber channels 6018 and 6022 open via, and the illuminator and collector optical fibers 4006 and 4010 (FIG. 4A) extend out from, the front-end surface 10000 of the front end 7002.

The blocking-member notch 7004 is configured to engage the blocking member 6032 of FIGS. 6 and 11. The notch 7004 has a front portion 7008 that extends all of the way through the back end 7000 and has a rear portion 7010 that extends only part way through the back end. The notch 7004 has an “L” shape (the portion 7008 is the bottom of the “L”) that complements the “L” shape of the blocking member 6032 (see FIG. 11) such that during insertion of the back end 7000 into the probe receptacle 2020, the blocking member eventually engages a back end 7012 of the portion 7010, which engagement acts to prohibit further insertion of the back end 7000 into the probe receptacle.

And the latch-engagement region 7006 is configured to engage a latch of the probe-connector clamping mechanism as described in conjunction with FIGS. 13 and 14. For example, while the probe connector 2008 is installed in the probe receptacle 2020, the latch presses against the latch-engagement region 7006 to hold the insertion back end 7000 stably and firmly (e.g., little or no wobble) in the receptacle so that the channels 6018 and 6022 become, and remain, optically aligned with the respective LEDS of the array 2044 (FIG. 6) and with the spectrometer input 6014 (FIG. 6), respectively.

Still referring to FIGS. 7-10, alternate embodiments of the probe connector 2008 are contemplated. For example, each of one or more of the insertion back end 7000, the front end 7002, the blocking-member notch 7004, and the latch-engagement region 7006, can have a respective suitable shape and respective suitable characteristics other than those described. Furthermore, embodiments described in conjunction with FIGS. 1-6 and 11-17 may be applicable to the probe connector 2008 of FIGS. 7-10.

FIG. 11 is a side view of the electromagnetic-energy blocking member 6032 of FIG. 6, according to an embodiment. The blocking member 6032 has a taller rear portion 11000 toward the inside of the base 2004 (FIG. 2) and has a shorter front portion 11002 toward the outside of the base such that the blocking member has an “L” shape. The rear portion 11000 of the blocking member 6032 is configured to engage the front portion 7008 of the blocking-member notch 7004 of the probe connector 2008 (FIGS. 7-10), and the front portion 11002 of the blocking member is configured to engage the portion 7010 of the blocking-member notch, while the probe connector is fully inserted within the probe receptacle 2020 (FIG. 6). While the probe connector 2008 is fully inserted and stably held within the probe receptacle 2020, the blocking member 6032 is configured to block electromagnetic energy leaking out of the illuminator optical fibers 4006 (FIG. 4) from leaking into the collector optical fiber 4010, and is configured to block electromagnetic energy leaking from the collector optical fiber from leaking into one or more of the illuminator optical fibers, thus limiting or eliminating crosstalk between the one or more illuminator optical fibers and the one or more collector optical fibers. A surface 11004 of the blocking member 6032 acts to stop further insertion of the probe connector 2008 into the probe receptacle 2020 when the surface 11004 engages the end surface 7012 of the notch 7004 (FIGS. 7-10). And, as described above, the blocking member 6032 can be formed form any suitable material that blocks electromagnetic energy in approximately the same wavelength range (e.g., approximately 500 nm-800 nm) as the electromagnetic energy radiated by the electromagnetic-energy generator 2022 (FIG. 2) with an attenuation factor of at least approximately 50%. Example suitable materials include plastic, metal, and ceramic.

Still referring to FIG. 11, alternate embodiments of the electromagnetic-energy blocking member 6032 are contemplated. For example, the blocking member 6032 can have any suitable shape other than an “L” shape. Furthermore, embodiments described in conjunction with FIGS. 1-10 and 12-17 may be applicable to the blocking member 6032 of FIG. 11.

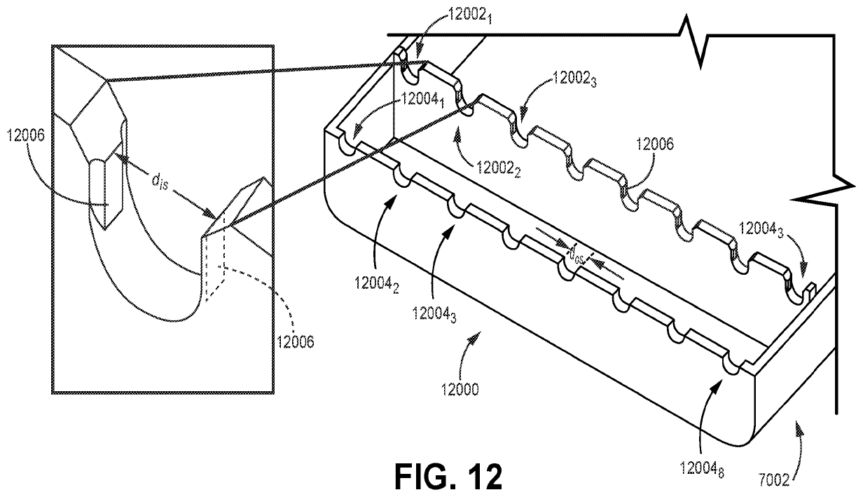

FIG. 12 is an isometric view, with a portion magnified, of an approximately half portion of a front 12000 of the front end 7002 of the probe connector 2008 of FIGS. 2, 3, and 6-10, according to an embodiment. The front end 7002 has a “clamshell” configuration, and the illustrated portion 12000 of the front end is one half of the clamshell. During manufacture of the probe connector 2008, after placement of the optical fibers 4006 and 4010 (FIG. 6) within one half of the clamshell, both halves of the clamshell are aligned, and thereafter are secured together in any suitable manner such as with adhesive or welding.

The portion 12000 includes inner fiber slots 12002₁-12002₈ and outer fiber slots 12004₁-12004₈, each pair of linearly aligned inner and outer fiber slots being configured to receive and to hold a respective optical fiber (optical fibers not shown in FIG. 12). That is, the pair of linearly aligned inner and outer fiber slots 12002₁ and 12004₁ is configured to hold the collector optical fiber 4010 (FIG. 6), the pair of linearly aligned inner and outer fiber slots 12002₂ and 12004₂ is configured to hold the illuminator optical fiber 4006 in the group 4000 (FIG. 6), the pairs of linearly aligned inner and outer fiber slots 12002₃₋₄ and 12004₃₋₄ are each configured to hold a respective illuminator optical fiber in the group 4002 (FIG. 6), and the pairs of linearly aligned inner and outer fiber slots 12002₅₋₈ and 12004₅₋₈ are each configured to hold a respective illuminator optical fiber in the group 4004 (FIG. 6).

Each inner fiber slot 12002 includes a respective pair of grippers 12006 configured to hold a respective one of the optical fibers to prevent damage to the probe connector 2008 (FIGS. 2-3B and 6-10) even as one (e.g., a moving subject) pulls on the fibers during normal use of the probe 2002 (FIG. 2). That is, the grippers 12006 are configured to counteract at least a majority of the pulling force that may be applied to the optical fibers 4006 and 4010 during normal use of the probe 2002. The grippers 12006 can be made from any suitable material such as plastic or rubber and are configured to counteract a pulling force having Cartesian (xyz) components up to approximately 46.0 Newtons (N) in the axial (along the fiber, e.g., z) dimension, 8.0 N side-to-side (e.g., x), and 8.0 N up-and-down (e.g., y).

Furthermore, each of the outer slots 12004 has a width dos in an approximate range of 1.7 mm-2.7 mm, for example 2.2 mm, and each of the inner slots 12002 has, between the grippers 12006, a width dis in an approximate range of 1.45 mm-2.45 mm, for example 1.95 mm.

Still referring to FIG. 12, alternate embodiments of the region 12000 of the probe-connector front 7002 are contemplated. For example, the region 12000 can have a configuration other than a clamshell configuration. Furthermore, the inner and outer slots 12002 and 12004 and the grippers 12006 can have dimensions other than the dimensions described. In addition, the other half of the clamshell can be similar to the half described but without the inner and outer fiber slots 12002 and 12004. Furthermore, embodiments described in conjunction with FIGS. 1-11 and 13-18 may be applicable to the region 12000 of FIG. 12.

FIG. 13 is a cutaway side view of the probe-connector receptacle 2020 and a probe-connector clamping mechanism 13000 in an open state without a probe connector 2008 installed in the receptacle, according to an embodiment.

FIG. 14 is a cutaway side view of a probe-connector receptacle 2020 and the clamping mechanism 13000 in a closed state with a probe connector 2008 installed and the mechanism holding the probe connector in position, according to an embodiment.

FIG. 15 is a top view of the probe connector 2008 (with some features omitted) and kinematic-alignment contact regions configured for contact with corresponding regions of the probe receptacle 2020 of FIG. 2, according to an embodiment.

Referring to FIGS. 13-14, in addition to the probe-connector sensor (a microswitch in the disclosed embodiment) 2040, the probe-connector clamping mechanism 13000 includes a connector-engagement latch 13002, a latch pivot rod 13004, a linkage arm 13006, linkage-arm pivot regions 13008 and 13010, a lever 13012, a lever pivot rod 13014, a bias spring 13016 (the one spring is shown in an open position 13018 (lighter shade) and in a closed position 13020 (darker shade) in FIGS. 13-14), a motor 13022 having a D-shaft 13024, a mechanism-open-close cam 13026, and six kinematic-alignment support regions, only two of which, 13028 and 13030, are shown in FIGS. 13-14.

Machine screws 13032 secure, to a bottom panel 13034 of the system housing (3002 of FIG. 3A and 3020 of FIG. 3B), a structure 13036, which forms the probe receptacle 2020 and at least a portion of the clamping mechanism 13000.

Referring to FIG. 15, the probe connector 2008 and the receptacle 2020 (FIGS. 13-14) are configured so that while the clamping mechanism 13000 is in the engaged state, the probe connector is stably and kinematically aligned with the LED array 2044 (FIG. 2) and the spectrometer input 6014 (FIG. 6) by contacting the inner walls of the receptacle at six kinematic-alignment contact regions 15000, 15002, 15004, 15006, 15008, and 15010. The contact region 15000 is located along a side of the insertion back end 7000 of the probe connector 2008, the contact regions 15002 and 15004 are located along the rear surface 6026 of the probe connector, and the contact regions 15006, 15008, and 15010 (shown in dashed line) are located along a side (e.g., bottom side) of the probe connector opposite the side in which the latch-engagement region 7006 is disposed. Referring to FIGS. 13-14, the receptacle 2020 can include, for each of one or more of the contact regions 15000, 15002, 15004, 15006, 15008, and 15010, a respective raised kinematic-contact-support region such as the regions 13028 and 13030. The locations of the contact regions can be determined conventionally and are related to the characteristics (e.g., dimensions, center of gravity, flexibility, positions of the optical-fiber channels 6018 and 6022) of the probe connector 2008.

Referring to FIGS. 13-15, operation of the probe-connector clamping mechanism 13000 is described, according to an embodiment.

The mechanism 13000 initially has the open state shown in FIG. 13.

Next, a user inserts the probe connector 2008 into the receptacle 2020 until the probe connector activates the sensor 2040; for example, if the sensor is a microswitch, then the user fully inserts the probe connector until the rear face 6026 (FIG. 8) of the probe connector pushes the microswitch button, thus activating the switch.

Then, in response to the sensor 2040 being activated, the control circuit 2028 (FIG. 2) activates the motor 13022 and causes the motor to rotate the cam 13026 from its disengaged position in FIG. 13 to its engaged position in FIG. 14.

As a result of the rotation of the cam 13026 from its disengaged position to its engaged position, the spring 13016, which is under tension, is allowed to contract from its disengaged (e.g., higher-tension) state 13018 to its engaged (e.g., lower-tension) state 13020.

The contracting spring 13016 pulls the bottom of the lever 13012 toward the spring. When fully contracted, the spring pulls the lever 13012 with a force in an approximate range of 70 Newtons (N)-90 N, for example 80 N. Furthermore, in its engaged position, the cam 13026 bears little to none of the force generated by the spring 13016; for example, although not shown, there can be a gap between the lever 13012 and the cam 13026 while the spring and the cam are in their engaged states so that the cam bears none of the spring force while the clamping mechanism 13000 holds the probe connector 2008 within the receptacle 2020.

In response to the contracting spring 13016 pulling the bottom of the lever 13012 toward the spring, the lever rotates counterclockwise around the pivot rod 13014 such that an upper portion of the lever forces the linkage member 13006 away from the spring.

The linkage member 13006 moving away from the spring 13016 and toward the receptacle 2020 forces the engagement latch 13002 to rotate about the pivot rod 13004 in a counterclockwise direction, and, therefore, causes the latch 13002 to engage the latch-engagement region 7006. Once engaged with the latch-engagement region 7006, the latch 13002 holds the optical-fiber channels 6018 and 6022 (e.g., FIG. 6) in a kinematically stable alignment with the LEDs of the array 2044 (FIG. 6) and with the spectrometer input 6014 (FIG. 6), respectively, by pressing each of the six kinematic-alignment contact regions 15000, 15002, 15004, 15006, 15008, and 15010 against a respective kinematic-contact-support region of one or more inner walls of the receptacle 2020 with a respective force; for example, the latch 13002 presses each of the contact regions 15008 and 15010 against the support regions 13028 and 13030, respectively, of the probe receptacle. Because the force pressing the contact region 15000 against an inner receptacle wall is orthogonal to the forces pressing the contact regions 15002 and 15004 against another inner receptacle wall and is orthogonal to the forces pressing the contact regions 15006, 15008, and 15010 against yet another inner receptacle wall, the pivot arm 13004 is not orthogonal to the page of FIGS. 13-14, but is canted from the orthogonal in the horizontal dimension by an angle in an approximate range of 2.5°-15°, for example in a range of 10°-15°. The horizontal angling of the pivot arm 13004 allows the engagement latch 13002 to generate forces in both horizontal dimensions as well as in the vertical dimension. Furthermore, the latch-engagement region 7006 has a side 15012 canted by an angle a relative to the orthogonal 15014 (the orthogonal 15014 is approximately parallel to the orthogonal relative to which the pivot arm 13004 is canted) to accommodate the canted pivot arm 13004; for example, the angle a can be approximately equal to the angle at which the pivot arm 13004 is canted.

To remove the probe connector 2008 from the receptacle 2020, a user activates the probe-connector release 2042 (FIG. 2), which may be a physical button of the interface 2034 (FIG. 2) or may be a virtual button displayed on the display 2032.

In response to a signal generated by the activated release 2042 (FIG. 2), the control circuit 2028 (FIG. 2) activates the motor 13022 and causes the motor to rotate the cam 13026 from its engaged position (FIG. 14) to its disengaged position (FIG. 13).

As a result of the rotation of the cam 13026 from its engaged position to its disengaged position, the cam 13026 forces the bottom of the lever 13012 in a direction toward the receptacle 2020, which stretches the spring from its engaged state 13020 to its disengaged state 13018.

The stretching spring 13016 pulls the bottom of the lever 13012 against the cam 13026 such that in its disengaged position, the cam 13026 bears most to all of the force generated by the spring 13016.

In response to the cam 13026 forcing the bottom of the lever 13012 away from the spring and toward the receptacle 2020, the lever rotates around the pivot rod 13014 in a clockwise direction such that an upper portion of the lever pulls the linkage member 13006 toward the spring 13016.

The linkage member 13006 moving toward the spring 13016 and away from the receptacle 2020 forces the engagement latch 13002 to rotate about the pivot rod 13004 in a clockwise direction, and, therefore, causes the latch 13002 to disengage the latch-engagement region 7006 of the probe connector 2008.

Once the latch 13002 is disengaged from the latch-engagement region 7006, a user is free to remove the probe connector 2008 from the receptacle 2020 with little or no resistance from the disengaged clamping mechanism 13000 or the inner walls of the receptacle 2020.

Still referring to FIGS. 13-15, alternate embodiments of the clamping mechanism 13000, the kinematic-alignment aspects of the probe connector 2008, and the operation of the clamping mechanism are contemplated. For example, the latch pivot arm 13004 can be canted vertically from the orthogonal to the pages of FIGS. 13-14 instead of, or in addition to, being canted horizontally. Furthermore, the clamping mechanism 13000 can be omitted, and the receptacle 2020 and probe connector 2008 can be designed so that the fit of the probe connector inside of the receptacle is “tight” enough so that once inserted into the receptacle, the probe connector is held within the receptacle so that the optical-fiber channels 6018 and 6022 are stably and kinematically aligned with the LEDs of the array 2044 (FIG. 2) and with the input to the spectrometer 6014 (FIG. 6). In addition, although described as being orthogonal to the pages of FIGS. 13-14, the pivot arm 13014 can be canted horizontally, vertically, or both horizontally and vertically relative to the orthogonal to the pages of FIGS. 13-14. Moreover, embodiments described in conjunction with FIGS. 1-12 and 16-18 may be applicable to the clamping mechanism 13000, the operation of the clamping mechanism, and the kinematic-alignment aspects of the probe connector 2008 of FIGS. 13-15.

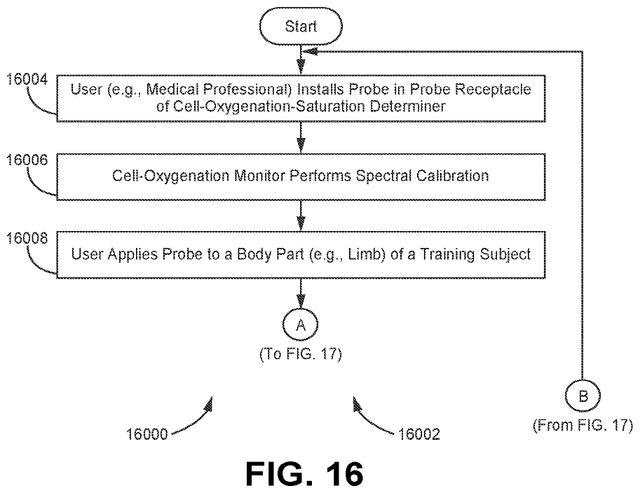

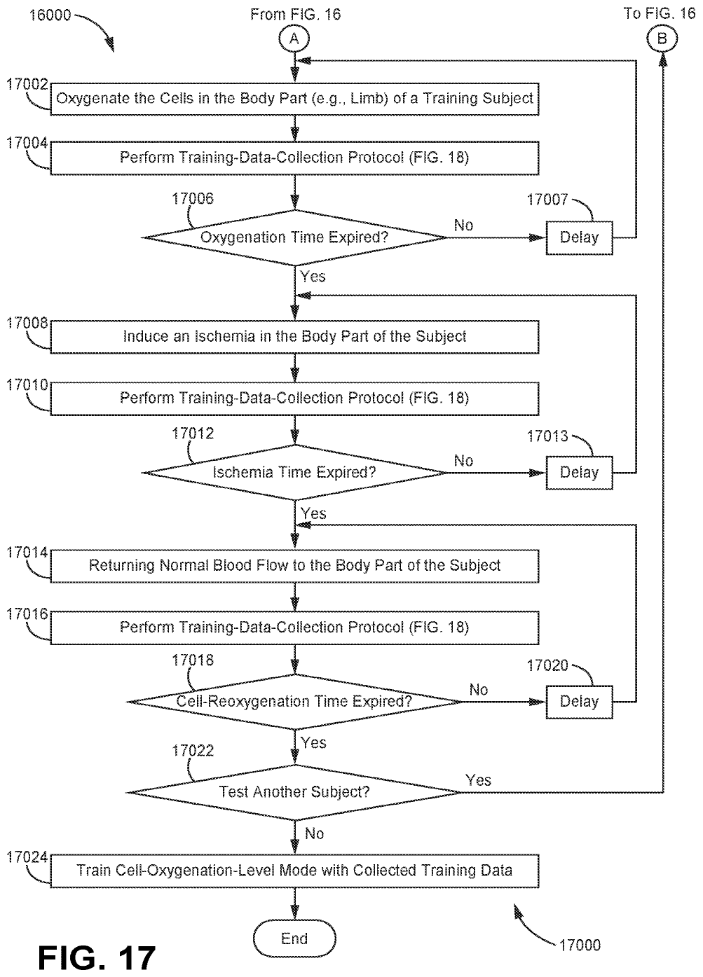

FIGS. 16-18 are respective portions 16002, 17000, and 18000 of a flow chart 16000 of a method for generating and collecting data for training a cell-oxygen-saturation-level learning-algorithm model, which is executable by the computing circuit 2048 (FIG. 2) for determining a cell-oxygen-saturation level in a human, according to an embodiment.

The training data is collected from human model-training subjects that are statistically diverse in at least the following physical traits: skin color (hereinafter “skin tone”) as measured, for example, by the Fitzpatrick scale, body-mass index (BMI), age, and biological sex. Other physical traits in which human model-training subjects can be statistically diverse include: size of body part (e.g., hand) from which the training data is generated, level of physical fitness, level of hydration, and general health (e.g., suffers from diabetes) during the time over which the training data is collected. For example, it is theorized that a suitable number of sets of training data can be collected from a total of approximately one hundred fifty diverse individual humans (e.g., individuals that are statistically diverse in at least skin tone, BMI, age, and biological sex).

Referring to FIGS. 2 and 16-18, the method represented by the flow chart 16000 is described, according to an embodiment.

Referring to FIG. 16, at step 16004, a user, such as a medical professional or trained data collector, installs the probe 2002 (FIGS. 2-3B) in the base 2004 (FIGS. 2-3B) by “plugging” the probe connector 2008 (FIGS. 13-14) into the probe-connector receptacle 2020 (FIGS. 13-14).

Next, at a step 16006, the control circuit 2028 performs a spectral calibration as described in conjunction with step 5006 of FIG. 5. Assuming that the control circuit 2028 detects no problems during the calibration routine (see step 5010 of FIG. 5), the control circuit proceeds to a step 16006.

At the step 16008 and as described in conjunction with step 5014 of FIG. 5, the user attaches the probe head 2010 to a body part (e.g., the thenar eminence of the subject's hand) from which the training data is being collected.

Next, referring to FIG. 17, at a step 17002 the user causes the cells, e.g., the skeletal muscle cells, in at least the body part to which the probe head 2010 is attached, to be oxygenated. For example, the user may attach, to the subject, a mask or cannula connected to a source of oxygen (O₂) in a concentration higher than the oxygen concentration normally found in air, for example, in a concentration in an approximate range of 30%-100%. Assuming that the subject is healthy, as he/she breathes in the higher concentration of oxygen, his/her cells become almost fully, to fully, saturated with oxygen. For example, the subject's cells attain an oxygen-saturation level in an approximate range of 95%-100%.

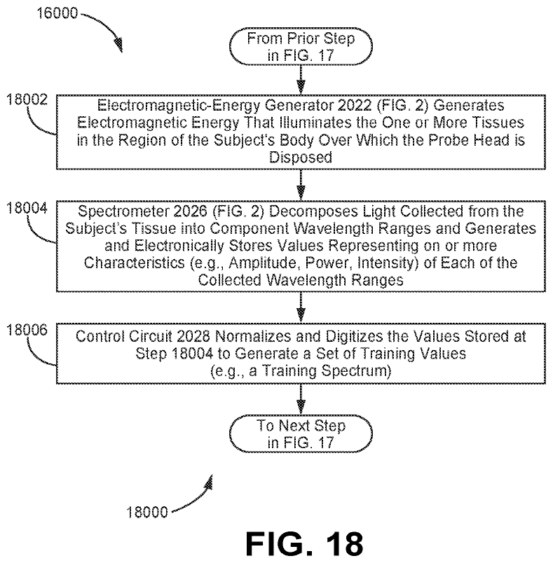

Then, at a step 17004, the control circuit 2028 controls the system 2000 to execute, or to otherwise perform, the training-data-collection protocol of FIG. 18.

Referring to FIGS. 4 and 18, at a step 18002, the control circuit 2028 activates the electromagnetic-energy generator 2022 such that the LEDS of the array 2044 sequentially radiate electromagnetic energy into the input ends of the illuminator optical fibers 4006 in the groups 4000, 4002, and 4004 (and possibly additional groups) for first, second, and third (and possibly subsequent) training illumination times, respectively, in a manner similar to that described in conjunction with step 5016 of FIG. 5.

During the first training illumination time, the radiated electromagnetic energy emanates from the output ends of the one or more illuminator optical fibers 4006 (only one such fiber shown in FIG. 4) in the group 4000 and illuminates the cells (e.g., skeletal muscle cells) in the region of the test subject's body over which the probe head 2010 is secured. Still during the first training illumination time, the illuminated cells redirect a portion of this electromagnetic energy for collection by the one or more collector optical fibers 4010 (only one collector optical fiber shown in FIG. 4), which direct the collected electromagnetic energy to the input 6014 (FIG. 6) of the spectrometer 2026.

Similarly, during the second training illumination time, the radiated electromagnetic energy emanates from the output ends of the one or more illuminator optical fibers 4006 (only two such fibers shown in FIG. 4) in the group 4002 and illuminates the cells (e.g., skeletal muscle cells) in the region of the test subject's body over which the probe head 2010 is secured.

Still during the second training illumination time, the illuminated cells redirect a portion of this electromagnetic energy for collection by the one or more collector optical fibers 4010 (only one collector optical fiber shown in FIG. 4), which direct the collected electromagnetic energy to the input 6014 (FIG. 6) of the spectrometer 2026.

And, during the third training illumination time, the radiated electromagnetic energy emanates from the output ends of the one or more illuminator optical fibers 4006 (only four such fibers shown in FIG. 4) in the group 4004 and illuminates the cells (e.g., skeletal muscle cells) in the region of the test subject's body over which the probe head 2010 is secured.

Still during the third training illumination time, the illuminated cells redirect a portion of this electromagnetic energy for collection by the one or more collector optical fibers 4010 (only one collector optical fiber shown in FIG. 4), which direct the collected electromagnetic energy to the input 6014 (FIG. 6) of the spectrometer 2026.

Next, at a step 18004, which can be similar to the step 5018 of FIG. 5 and which is concurrent with the step 18002, the spectrometer 2026 decomposes, into wavelength ranges, the redirected electromagnetic energy, which is collected, and which is provided to the spectrometer input, by the group 4008 of one or more collector optical fibers 4010 (one collector optical fiber in the described embodiment). For example, in an embodiment in which the wavelengths in the spectra of the redirected electromagnetic energy span an approximate range of 501 nm-850 nm, the spectrometer 2026 may decompose the collected electromagnetic energy into the following thirty-five approximate wavelength ranges: 501 nm-510 nm, 511 nm-520 nm, 521 nm-530 nm, . . . , 831 nm-840 nm, and 841 nm-850 nm.

And, for each wavelength range, the spectrometer 2026 generates one or more values respectively related to one or more characteristics of the wavelengths in the wavelength range. For example, the spectrometer generates a value related to the combined intensity (e.g., magnitude, amplitude, power) of the wavelengths in the wavelength range. Further in example, the spectrometer 2026 generates, as a representative of each value, a respective analog electrical signal, and converts the analog signal into a corresponding digital signal. Still further in example, the spectrometer 2026 generates a number of digital samples of each characteristic for each wavelength range, and mathematically combines (e.g., averages) these samples to generate, for each wavelength range, a respective value for each analyzed spectral characteristic.

For purposes of clarity, in the following description the spectrometer 2026 generates, for each wavelength range, only values only for intensity, it being understood, however, that the spectrometer can generate, for each wavelength range, values for other wavelength-range characteristics instead of, or in addition to, intensity.

Still at the step 18004, during the first training illumination time, for each wavelength range of the collected electromagnetic energy, the spectrometer 2026 generates a respective value related to intensity as described above, and the control circuit 2028 stores the respective value in the memory 2030 such that the value is associated with the wavelength range and the radius r₁ of the group 4000 of one or more (here one) illuminator optical fibers 4006.

During the second training illumination time, for each wavelength range of the collected electromagnetic energy, the spectrometer 2026 generates a respective value related to intensity, and the control circuit 2028 stores the respective value in the memory 2030 such that the value is associated with the wavelength range and the radius r₂ of the group 4002 of one or more (here two) illuminator optical fibers 4006.

During the third training illumination time, for each wavelength range of the collected electromagnetic energy, the spectrometer 2026 generates a respective value related to intensity, and the control circuit 2028 stores the respective value in the memory 2030 such that the value is associated with the wavelength range and the radius r₃ of the group 4004 of one or more (here four) illuminator optical fibers 4006.

And during each subsequent training illumination time, if any, for each wavelength range of the collected electromagnetic energy, the spectrometer 2026 generates a respective value related to intensity, and the control circuit 2028 stores the value in the memory 2030 such that the value is associated with the wavelength range and the radius rₙ of the respective group of one or more illuminator optical fibers 4006.

As described above, the number of illuminator optical fibers 4006 in a group increases with increasing radius r such that the level (e.g., the number of photons) of electromagnetic energy that the one or more (here one) collector optical fibers 4010 collect during each of the first, second, third, and any subsequent, illumination periods is approximately equal to the levels of electromagnetic energy collected during the other illumination periods, or has at least a minimum-threshold S/N.

Next, at a step 18006, the computing circuit 2048 digitizes and stores, in the memory 2030, values corresponding to one or more characteristics (e.g., intensity) of each of the collected wavelength ranges, which values together compose a respective training spectrum. The computing circuit 2048 normalizes each of the values stored in the memory 2030 during the first, second, third, and any subsequent, training illumination times at the step 18004 to generate the values that compose the training spectrum. For each stored value being normalized, the computing circuit 2048 first divides the corresponding calibration value, previously determined and stored in the memory 2030 at the step 16006, by the stored value. For example, if “200” is the decimal representation of the digital intensity value stored for the wavelength range 501 nm-510 nm at radial distance r₁ during the first training illumination time and “300” is the decimal representation of the corresponding digital intensity calibration value stored for the same wavelength range and same radial distance during the calibration procedure, then the computing circuit 2048 generates the quotient 300/200=3/2=1.50. Next, the computing circuit 2048 generates the normalized version of the value “200” for the wavelength range 501 nm-510 nm at the radial distance r₁ for the first illumination time equal to log₁₀(1.50)=0.18. Alternatively, the computing circuit 2048 generates the normalized version equal to |log₁₀(1.50)|=0.18. The computing circuit 2048 normalizes each of the other values stored in the memory 2030 during the first, second, third, and any subsequent, training illumination times in a similar manner. Normalizing the values can reduce the magnitude of, or altogether eliminate, errors caused by shifts in the spectrum generated the electromagnetic-energy generator 2022 due to, for example, aging of the LEDs in the array 2044, shifts in temperature of, or supply voltage provided to, the generator from one training subject to the next, and differences in electromagnetic-energy illuminating and collecting from the probe 2002 to probe during the training procedure. As described above, the set of stored normalized intensity values for all of the wavelength ranges and radii over a set of illumination times constitutes a single set of training data representing heavily oxygenated cells, data that one can use to train the cell-oxygenation-level-determining model to be executed by the computing circuit 2048 to accurately determine the cell-oxygenation levels of heavily oxygenated cells.

Referring to again to FIG. 17, then, at a step 17006, the control circuit 2028 determines whether the time for oxygenating the body part of the training subject is expired; for example, the control circuit can include a hard-wired clock, or can execute program instructions that cause the control circuit to function as a clock, for “counting down” a set oxygenation time, which can be, for example, in an approximate range of 1.0-20.0 minutes. If the oxygenation time has not expired, then, at a step 17007, the control circuit 2028 implements a delay in the approximate range of 0-60 seconds and, after the expiration of the delay, returns to the step 17002 to generate another set of training data representing cells with oxygenations that are increasing from normal to high levels or that are stable at high levels. But if the oxygenation time has expired, then the training method proceeds to a step 17008.

At the step 17008, the training-data-collection technician induces an ischemia in the subject's body part to which the probe head 2010 (FIGS. 2 and 4) is attached. For example, if the probe head 2010 is attached to the thenar eminence of the model-training-subject's hand, then the technician wraps a blood-pressure cuff of a sphygmomanometer around the subject's arm to which the hand is attached and inflates the cuff to cut off blood flow to the hand. The training method then proceeds to a step 17010.

Next, at the step 17010, the control circuit 2028 again performs the steps 18002, 18004, and 18006 of FIG. 18 to generate, and to store in the memory 2030, a set of training data representing cells having oxygenations that are decreasing from high (e.g., 95%-100%) levels to low levels (e.g., 0%-50%) and that are stable at the low levels, in a manner similar to that described in conjunction with the step 17004.

Then, at a step 17012, the control circuit 2028 determines whether the time for inducing an ischemia in the body part of the training subject is expired; for example, the control circuit 2028 can include a hard-wired clock, or can execute program instructions that cause the control circuit to function as a clock, for “counting down” a set ischemia-inducing time, which can be, for example, in an approximate range of 1.0-20.0 minutes. If the ischemia-inducing time has not expired, then, at a step 17013, the control circuit 2028 implements a delay in the approximate range of 0-60 seconds and, after the expiration of the delay, returns to the step 17008 to generate another set of training data representing cells with oxygenations that are decreasing from high to low high levels or that are stable at low (oxygen-deprived) levels. But if the ischemia-inducing time has expired, then the training method proceeds to a step 17014.

At the step 17014, the training-data-collection technician removes the cause of the ischemia induced at the step 17008 to restore normal blood flow to the subject's body part to which the probe head 2010 (FIGS. 2 and 4) is attached. For example, if the probe head 2010 is attached to the thenar eminence of the training-subject's hand and, at the step 17008, the technician wrapped a blood-pressure cuff of a sphygmomanometer around the subject's arm to which the hand is attached, the technician deflates the cuff to restore blood flow to the hand while the training subject is breathing normal air. The training method then proceeds to a step 17016.

Next, at the step 17016, the control circuit 2028 again performs the steps 18002, 18004, and 18006 of FIG. 18 to generate, and to store in the memory 2030, a set of training data representing cells with oxygenations that are increasing from low levels (oxygen-deprived) to normal levels (e.g., 88%-95%) or that are stable at normal levels, in a manner similar to that described in conjunction with the step 17004.

Then, at a step 17018, the control circuit 2028 determines whether the time for re-oxygenating the body part of the training subject is expired; for example, the control circuit 2028 can include a hard-wired clock, or can execute program instructions that cause the control circuit to function as a clock, for “counting down” a set reoxygenation time, which can be, for example, in an approximate range of 1.0-20.0 minutes. If the cell-reoxygenation time has not expired, then, at a step 17020, the training method waits a delay in an approximate range of 0-60 seconds, and returns to the step 17014 to generate another set of training data representing reoxygenating cells. But if the cell-reoxygenation time has expired, then the training method proceeds to a step 17022.

At the step 17022, the training technician, or the control circuit 2028, determines whether training data is to be generated using another test subject. If the technician or the control circuit 2028 determines that training data is to be generated using another test subject, then technician removes the probe head 2010 from, and otherwise releases, the test subject, removes the probe connector 2008 from the receptacle 2020 and disposes of the probe 2002, and the training method returns to the step 16004 (FIG. 16). But if the technician or the control circuit 2028 determines that no additional training data is to be generated, then the training method proceeds to a step 17024.

At the step 17024, one (not necessarily the training technician) trains a learning-algorithm model to be executed by the computing circuit 2048 with the generated and stored (in the memory 2030, at least initially) training data according to any suitable training protocol, which can be conventional. The training at the step 17024 need not be done within any particular time after the step 17022 and may be considered to be a method that is not part of the method represented by the flow chart 16000. For example, the stored training data may be transported to a location remote from the training-data-collection site, such as a computer laboratory, for training the algorithm model.

Referring to FIGS. 16-18, the training data includes spectra that occur during all potential cell-oxygenation and cell-deoxygenation events that a patient or other subject, with a set of physical traits within a wide range, may experience. By appropriately setting the oxygenation, ischemia, and cell-reoxygenation times per steps 17006, 17012, and 17018, the described training method generates and collects training data that represents cells as their oxygenations increase from normal levels to high levels, remain stable at high levels, decrease from high levels to low levels, remain stable at low levels, increase from low levels to normal levels, and remain stable at normal levels. Furthermore, by testing a statistically suitable number of subjects with statistically diverse traits such as age, biological sex, skin tone, and BMI, the volume and diversity of the training data is suitable to train an algorithm model to be executed by the computing circuit 2048 such that the cell-oxygenation-level determining-and-monitoring system 2000 (FIG. 2) can accurately determine and monitor cell-oxygenation levels for most or all patients experiencing most or all known medical events. For example, testing approximately 100-200 subjects, such as approximately 150 subjects, according to the method and subject diversity described in conjunction with FIGS. 16-18, can yield a number of diverse training spectra in the approximate range of 50,000-2,000,000, which number is sufficient to train the algorithm model so that it does not exhibit bias, at least not with respect to the traits that are statistically diverse among the test subjects.

Still referring to FIGS. 16-18, alternate embodiments of the training-data-generating-and-collecting method are contemplated. For example, one or more of the steps 17002, 17008, and 17014 can be omitted from the method. Furthermore, instead of collecting training data for, and training, one algorithm model, one may collect training data for, and train, multiple algorithm models, one model for each of multiple body traits. For example, there can be a respective model for each skin tone as defined by Fitzpatrick skin type, for each of multiple BMI ranges, for each biological sex, or for each of multiple age ranges. In such an embodiment, the control circuit 2028 (FIG. 2) can be configured to generate a model-selection menu on the display 2032 (FIG. 2) so that a user can select the best model for a particular subject. Moreover, embodiments described in conjunction with FIGS. 1-15 may be applicable to the training-data-generating-and-collecting method of FIGS. 16-18.

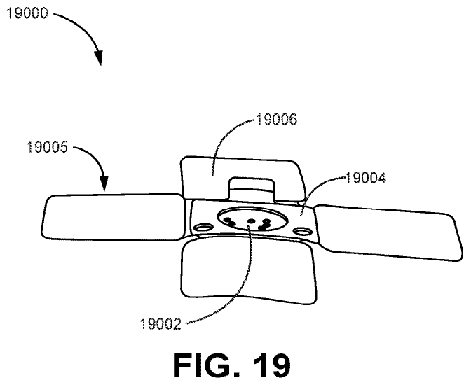

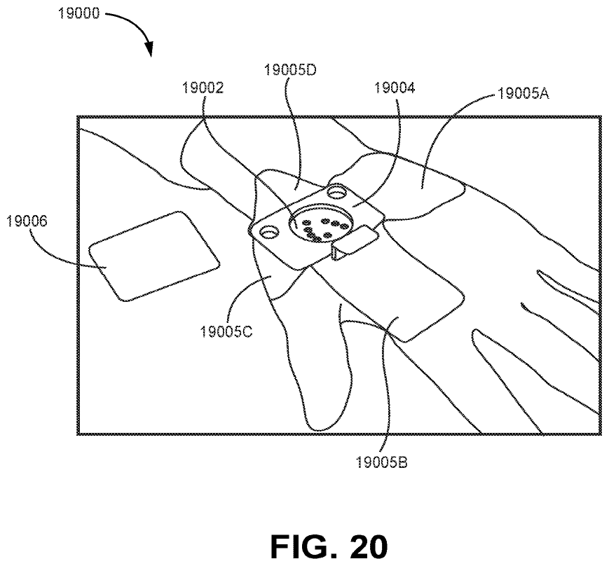

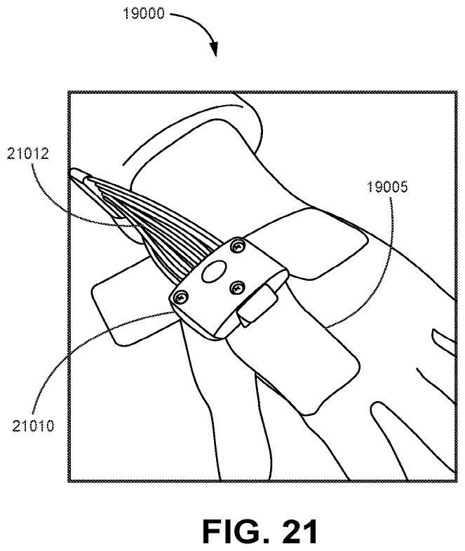

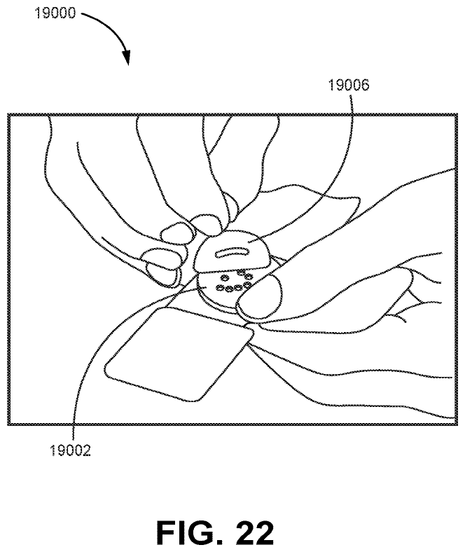

FIGS. 19-22 are views of alternative embodiments of an attachment member configured for secure attachment of a probe head to a portion of a human hand such as the thenar eminence. FIG. 19 is a view of an attachment member 19000, which is un-attached to a probe head (such as the probe head 2010 of FIG. 2) of a probe (such as the probe 2002 of FIG. 2). FIG. 20 is a view of the attachment member 19000 secured to the thenar eminence of a human hand. FIG. 21 is a perspective view of a probe head 21010 attached to the attachment member 19000 while the attachment member 19000 is secured to the thenar eminence of a human hand. And FIG. 22 is a view of an attachment member 19000 with a protective film 19006 disposed over the attachment member 19000, according to an embodiment.

Referring to FIG. 19, the attachment member 19000 includes a receptacle 19004 having a bottom 19002, according to an embodiment. The receptacle 19004 is coupled to a plurality of adhesive surfaces 19005 (four of which are shown). Each adhesive surface 19005 is coupled to one side of the receptacle 19004 orthogonal to another adhesive surface 19005. Each adhesive surface 19005 is also initially covered with a protective film 19006. The protective films 19006 are configured to be removed from the adhesive surface 19005, for example, by peeling the protective film 19006 and exposing the adhesive underneath. Each adhesive surface 19005 then engages with a portion of the human hand. The receptacle 19004 is configured to engage with the probe head of a probe, for example, with the probe head 2010 of the probe 2002 of FIG. 2. Referring to FIG. 22, the attachment member 19000 also includes a protective film 19006 disposed over the bottom 19002 of the receptacle 19004 and configured to be removed prior to attaching the probe head to the receptacle 19004.

Referring to FIG. 20, the attachment member 19000 is engaged to the thenar eminence of a human hand. After removing the protective film 19006 covering the adhesive surfaces 19005, attachment member 19000 is engaged with the thenar eminence by securing the adhesive surfaces 19005 to different portions of the hand proximate to the thenar eminence. For example, one adhesive surface 19005A can be attached horizontally across the palm, and another adhesive surface 19005B can be attached vertically upwards towards the index finger. Furthermore, the adhesive surfaces 19005 are sufficiently flexible to bend or shape around the contours of the surface of the hand. A third adhesive surface 19005C engages with the hand by wrapping around the base of the thumb. Another adhesive surface 19005D is attached to the base of the palm area and may extend to the wrist. Having the adhesive surfaces 19005 engage with the hand in the above-described manner typically provides a secure fastening of the attachment member 19000 to the hand, and, therefore, typically allows the sensing measurements to be suitably accurate.

Referring to FIG. 21, once secured to the thenar eminence, a probe head 21010 can be secured to the attachment member 19000. For example, one can secure the probe head 21010 by twisting or “clicking” the probe head into the receptacle 19004. The probe head 21010 is coupled to an umbilical cord 21012 that is configured to couple electromagnetic energy to a base as previously described. For example, the probe head 21010, umbilical cord 21012, and base can be the same as, or similar to, the probe head 2010, umbilical cord 2006, and base 2004, respectively, of FIG. 2.

Still referring to FIGS. 19-22, alternate embodiments of the attachment member 19000 are contemplated. For example, embodiments described in conjunction with FIGS. 1-18 may be applicable to the attachment member 19000 of FIGS. 19-22.

### Example Embodiments

Example 1 includes a system, comprising: a housing; an electromagnetic unit disposed in the housing and configured: to generate electromagnetic energy during a time; and to direct the electromagnetic energy into a body having at least one muscle cell; an optical sensor disposed in the housing and configured to receive a portion of the electromagnetic energy redirected by the body and to convert the received portion of the electromagnetic energy into a signal; and a computing circuit disposed in the housing, coupled to the electromagnetic unit and the optical sensor, and configured to determine, in response to the signal, a level of oxygenation of one or more of the at least one muscle cell.

Example 2 includes the system of any of Examples 1-2 wherein the electromagnetic unit includes at least one light-emitting diode.

Example 3 includes the system of Example 1, wherein the electromagnetic unit includes at least one light-emitting diode each configured for generating at least one wavelength in an approximate range of 400 nm-900 nm and having a first intensity and at least one wavelength in an approximate range of 400 nm-900 nm and having a second intensity that is less than the first intensity.

Example 4 includes the system of any of Examples 1-4, wherein the electromagnetic unit includes at least one light-emitting diode each configured for generating at least one wavelength in a range of 400-900 nm.

Example 5 includes the system of Example 1, wherein the electromagnetic unit includes a linear arrangement of multiple light-emitting diodes.

Example 6 includes the system of any of Examples 1-5, wherein the electromagnetic unit includes: light-emitting diodes; and a drive circuit configured for activating and powering, selectively, the light-emitting diodes.

Example 7 includes the system of any of Examples 1-6, wherein the electromagnetic unit includes: light-emitting diodes; and a temperature-control circuit configured to maintain a respective temperature of each of the light-emitting diodes within a temperature range.

Example 8 includes the system of any of Examples 1-8, wherein the optical sensor includes a spectrometer configured: to receive the redirected portion of the electromagnetic energy; and to generate, for each of at least one wavelength range in the redirected portion of the electromagnetic energy, a respective electronic signal related to a value of a characteristic of the at least one wavelength range.

Example 9 includes the system of Example 1, further comprising a housing configured to directly attach to a body, wherein the electromagnetic unit, the optical sensor, and the computing circuit are disposed in the housing.

Example 10 includes the system of any of Examples 1-9, wherein the computing circuit is configured for determining a level of oxygenation of one or more of the at least one muscle cell in response to a respective value of a characteristic of each of at least one wavelength range of the portion of redirected electromagnetic energy.

Example 11 includes the system of any of Examples 1-10, wherein the computing circuit is configured: for implementing a machine-learning algorithm; and for determining a level of oxygenation of one or more of the at least one muscle cell by providing, as at least one input to the implemented machine-learning algorithm, a respective value of a characteristic of each of at least one wavelength range of the portion of redirected electromagnetic energy.

Example 12 includes the system of any of Examples 1-11, wherein the computing circuit is configured to implement a locally weighted regression model; and to determine a level of oxygenation of one or more of the at least one muscle cell by providing, as at least one input to the implemented locally weighted regression model, a respective value of a characteristic of each of at least one wavelength range of the portion of redirected electromagnetic energy.

Example 13 includes a probe, comprising: a head securable to a body having muscle cells; a collector optical fiber having a collector end disposed on the head; at least one first illuminator optical fiber each having a respective first illuminator end disposed on the head approximately a first distance from the collector end; at least one second illuminator optical fiber each having a respective second illuminator end disposed on the head approximately a second distance from the collector end; and a connector configured for coupling opposite ends of the collector optical fiber, the at least one first illuminator optical fiber, and the at least one second illuminator optical fiber, to a device configured for determining a level of oxygenation of at least one of the muscle cells.

Example 14 includes the probe of Example 13, wherein the head includes an adhesive configured to adhere the head to the body.

Example 15 includes the probe of any of Examples 13-14, comprising at least one third illuminator optical fiber each having a respective third illuminator end disposed on the head approximately a third distance from the collector end, wherein the connector is configured for coupling the at least one third illuminator optical fiber to the device configured for determining a level of oxygenation of at least one of the muscle cells.

Example 16 includes the probe of any of Examples 14-15, wherein the head is configured to be detachable and reattachable to a receptacle while the receptacle is attached to the body.

Example 17 includes the probe of any of Examples 13-16, wherein the head is securable to a thenar eminence of a human hand.

Example 18 includes the probe of any of Examples 13-17, wherein a protective film is disposed between the collector end and the body.

Example 19 includes the probe of any of Examples 13-18, wherein a number of the at least one first illuminator optical fiber is an integer multiple of a number of the at least one second illuminator optical fiber.

Example 20 includes the probe of any of Examples 13-19, wherein a ratio of a number of the at least one first illuminator optical fiber to the first distance is approximately equal to a number of the at least one second illuminator optical fiber to the second distance.

Example 21 includes the probe of any of Examples 13-20, further comprising a calibrator.

Example 22 includes the probe of any of Examples 13-21, further comprising an authenticator.

Example 23 includes the probe of any of Examples 13-22, wherein the connector includes a blocking slot separating at least a portion of the collector optical fiber from at least a portion of the at least one first illuminator optical fiber and at least a portion of the at least one second illuminator optical fiber.

Example 24 includes the probe of any of Examples 13-23, further comprising an electromagnetic-energy generator.

Example 25 includes a system, comprising: a probe configurable for directing electromagnetic energy into a body having at least one muscle cell, and for receiving a portion of the electromagnetic energy redirected by the body during a time; and a base configured for coupling with the probe and including a generator configured for generating the electromagnetic energy during the time, and an optical sensor configured to receive the portion of the redirected electromagnetic energy and to convert the portion into a signal, and a computing circuit configured for determining, in response to the signal, a level of oxygenation of one or more of the at least one muscle cell.

Example 26 includes the system of Example 25, wherein the generator includes at least one light-emitting diode.

Example 27 includes the system of any of Examples 25-26, wherein the generator includes at least one light-emitting diode each configured for generating at least one wavelength in an approximate range of 400 nm-900 mn having a first intensity and at least one infrared wavelength in an approximate range of 400 nm-900 nm having a second intensity that is less than the first intensity.

Example 28 includes the system of any of Examples 25-27, wherein the generator includes at least one light-emitting diode each configured for generating at least one wavelength in a range of 400-900 nm.

Example 29 includes the system of any of Examples 25-28, wherein the generator includes light-emitting diodes each configured to generate electromagnetic energy across a respective spectrum that includes wavelengths in an approximate range of 400 nm-900 nm and that is approximately equal to each respective spectrum generated by another one or more of the light-emitting diodes.

Example 30 includes the system of any of Examples 25-29, wherein the probe is configured to direct the electromagnetic energy into the body at one or more distances from where the probe receives the portion of the redirected electromagnetic energy.

Example 31 includes the system of any of Examples 25-30, wherein the electromagnetic energy is directed into or out of the body via optical fibers at one or more distances from where light is received.

Example 32 includes the system of any of Examples 25-31, wherein the generator includes an arrangement of seven light-emitting diodes.

Example 33 includes the system of any of Examples 25-32, wherein the generator includes: light-emitting diodes; and a drive circuit configured for activating and powering, selectively, the light-emitting diodes.

Example 34 includes the system of any of Examples 25-33, wherein the generator includes: light-emitting diodes; and a temperature-control circuit configured to maintain a respective temperature of each of the light-emitting diodes within a temperature range.

Example 35 includes the system of any of Examples 25-34, further comprising: wherein the generator includes light-emitting diodes; wherein the probe includes a connector housing ends of optical fibers; and a receptacle configured for receiving the connector and for aligning each of the ends of the optical fibers with a respective one of the light-emitting diodes.

Example 36 includes the system of any of Examples 25-35, further comprising: wherein the generator includes light-emitting diodes; wherein the probe includes a connector housing ends of optical fibers; and a receptacle assembly including a receptacle configured for receiving the connector, a latch, and a motor configured for aligning each of the ends of the optical fibers with a respective one of the light-emitting diodes by causing the latch to engage the connector.

Example 37 includes the system of Example 36, wherein the motor is configured for releasing the connector for removal from the receptacle by causing the latch to disengage the connector.

Example 38 includes the system of any of Examples 25-37, wherein: the generator includes light-emitting diodes; the probe includes a connector housing ends of optical fibers; and the base includes a receptacle configured for receiving the connector, a latch, a first sensor configured for detecting the connector in the receptacle, and a motor configured for aligning each of the ends of the optical fibers with a respective one of the light-emitting diodes by causing the latch to engage the connector in response to the optical sensor detecting the connector in the receptacle.

Example 39 includes the system of Example 38, wherein the motor is configured for releasing the connector for removal from the receptacle by causing the latch to disengage the connector in response to a second sensor.

Example 40 includes the system of Example 39, wherein the second sensor includes an electronic switch.

Example 41 includes the system of any of Examples 25-40, wherein: the generator includes light-emitting diodes; the probe includes a connector housing ends of optical fibers and a latch-engagement region; and the base includes a receptacle having contact regions and configured to receive the connector, a latch, and a motor configured for aligning each of the ends of the optical fibers with a respective one of the light-emitting diodes by causing the latch to engage the latch-engagement region to force the connector against the contact regions.

Example 42 includes the system of Example 41, wherein the motor is further configured for releasing the connector for removal from the receptacle by causing the latch to disengage the latch-engagement region.

Example 43 includes the system of any of Examples 41-42, further comprising: wherein the generator includes light-emitting diodes; wherein the probe includes a connector housing ends of optical fibers; and a receptacle assembly including a receptacle configured for receiving the connector, a latch, and a motor configured for aligning each of the ends of the optical fibers with a respective one of the light-emitting diodes by causing the latch to engage the connector, wherein the motor is further configured for releasing the connector for removal from the receptacle by causing the latch to disengage the latch-engagement region.

Example 44 includes the system of any of Examples 25-43, wherein the optical sensor includes a spectrometer configured: for receiving, from the probe, the redirected portion of the electromagnetic energy; and for generating, for each of at least one wavelength range in the redirected portion of the electromagnetic energy, a respective electronic signal related to a value of a characteristic of the at least one wavelength range.

Example 45 includes the system of any of Examples 25-44, wherein the optical sensor includes a spectrometer configured: for receiving, from the probe, the redirected portion of the electromagnetic energy; and for generating, for each of at least one wavelength range in the redirected portion of the electromagnetic energy, a respective electrical signal related to an intensity of one or more wavelengths present in the at least one wavelength range.

Example 46 includes the system of any of Examples 25-45. wherein: the generator includes light-emitting diodes; the probe includes a connector housing ends of optical fibers; and the base further includes a spectrometer having an input configured for receiving, from the probe, the redirected portion of the electromagnetic energy, and configured for generating, for each of at least one wavelength range in the redirected portion of the electromagnetic energy, a respective electronic signal related to a value of a characteristic of the at least one wavelength range, and a receptacle configured for receiving the connector, for aligning each of the ends of some of the optical fibers with a respective one of the light-emitting diodes, and for aligning each of the ends of at least one other of the optical fibers with the spectrometer input.

Example 47 includes the system of any of Examples 25-46, wherein: the generator includes light-emitting diodes; the probe includes a connector housing ends of optical fibers; and the base further includes a spectrometer having an input configured for receiving, from the probe, the redirected portion of the electromagnetic energy, and configured for generating, for each of at least one wavelength range in the redirected portion of the electromagnetic energy, a respective electronic signal related to a value of a characteristic of the at least one wavelength range; a receptacle configured for receiving the connector, configured for aligning each of the ends of ones of the optical fibers with a respective one of the light-emitting diodes, configured for aligning each of the ends of at least one other of the optical fibers with the spectrometer input, and having a structure configured for impeding coupling of light between the spectrometer input and an output of the electromagnetic-energy generator while the optical fibers are respectively aligned with the light-emitting diodes and while the at least one other of the optical fibers is aligned with the spectrometer input.

Example 48 includes the system of any of Examples 25-47, wherein: the generator includes light-emitting diodes; the probe includes a connector housing ends of optical fibers and having a slot between a first set of the optical fibers and a second set of at least one of the optical fibers; and the base includes a spectrometer having an input configured for receiving, from the probe, the redirected portion of the electromagnetic energy, and configured for generating, for each of at least one wavelength range in the redirected portion of the electromagnetic energy, a respective electronic signal related to a value of a characteristic of the at least one wavelength range, and a receptacle configured for receiving the connector, configured for aligning each of the ends of the optical fibers of the first set with a respective one of the light-emitting diodes, configured for aligning each of the ends of the at least one optical fiber of the second set with the spectrometer input, and including an electromagnetic-radiation shield configured for disposal in the slot.

Example 49 includes the system of any of Examples 25-48, wherein the computing circuit is configured to control the generator.

Example 50 includes the system of any of Examples 25-49, wherein: the probe comprises an authenticator; and the computing circuit is configured for determining, in response to the authenticator, whether the probe is authorized for use with the base.

Example 51 includes the system of any of Examples 25-50, wherein: the probe comprises an authenticator; and the computing circuit is configured for determining, in response to the authenticator, whether the probe is suitable for coupling with the generator, and for disabling, in response to determining that the probe is unsuitable, the base from functioning if the probe is installed in the base.

Example 52 includes the system of any of Examples 25-51, wherein the computing circuit is configured for determining a level of oxygenation of one or more of the at least one muscle cell in response to a respective value of a characteristic of each of at least one wavelength range of the portion of redirected electromagnetic energy.

Example 53 includes the system of any of Examples 25-52, wherein the computing circuit is configured for implementing a machine-learning algorithm; and for determining a level of oxygenation of one or more of the at least one muscle cell by providing, as at least one input to the implemented machine-learning algorithm, a respective value of a characteristic of each of at least one wavelength range of the portion of redirected electromagnetic energy.

Example 54 includes the system of any of Examples 25-53, wherein the computing circuit is configured for implementing a mathematical algorithm; and for determining a level of oxygenation of one or more of the at least one muscle cell by providing, as at least one input to the implemented mathematical algorithm, a respective value of a characteristic of each of at least one wavelength range of the portion of redirected electromagnetic energy.

Example 55 includes the system of any of Examples 25-54, wherein the computing circuit is configured for implementing a mathematical model; and for determining a level of oxygenation of one or more of the at least one muscle cell by providing, as at least one input to the implemented mathematical model, a respective value of a characteristic of each of at least one wavelength range of the portion of redirected electromagnetic energy.

Example 56 includes the system of any of Examples 25-55, wherein the computing circuit is configured for implementing a locally weighted regression model; and for determining a level of oxygenation of one or more of the at least one muscle cell by providing, as at least one input to the implemented locally weighted regression model, a respective value of a characteristic of each of at least one wavelength range of the portion of redirected electromagnetic energy.

Example 57 includes an apparatus, comprising: a generator configured to provide wavelengths electromagnetic energy in an approximate range of 400 nm-900 nm to a probe configurable to direct the electromagnetic energy into a body having at least one muscle cell, and to collect a portion of the electromagnetic energy redirected by the body over a time during which the generator provides the electromagnetic energy; and a computing circuit configured to determine, in response to the portion of redirected electromagnetic energy, a level of oxygenation of one or more of the at least one muscle cell.

Example 58 includes the apparatus of Example 57, wherein the generator includes at least one light-emitting diode.

Example 59 includes the apparatus of any of Examples 57-58, wherein the generator includes at least one light-emitting diode each configured for generating at least one wavelength having a first intensity and at least one wavelength having a second intensity that is less than the first intensity.

Example 60 includes the apparatus of any of Examples 57-59, wherein the generator includes at least one light-emitting diode each configured for generating at least one visible wavelength and at least one infrared wavelength.

Example 61 includes the apparatus of any of Examples 57-60, wherein the generator includes light-emitting diodes each configured to generate electromagnetic energy across a respective spectrum that is approximately equal to each respective spectrum generated by another one or more of the light-emitting diodes.

Example 62 includes the apparatus of any of Examples 57-61, wherein the generator includes a row of seven light-emitting diodes.

Example 63 includes the apparatus of any of Examples 57-62, wherein the generator includes: light-emitting diodes; and a drive circuit configured for activating and powering, selectively, the light-emitting diodes.

Example 64 includes the apparatus of any of Examples 57-63, wherein the generator includes: light-emitting diodes; and a temperature-control circuit configured to maintain a respective temperature of each of the light-emitting diodes within a temperature range.

Example 65 includes the apparatus of any of Examples 57-64, further comprising: wherein the generator includes light-emitting diodes; and a receptacle configured for receiving a probe connector housing ends of optical fibers and for aligning each of the ends of the optical fibers with a respective one of the light-emitting diodes.

Example 66 includes the apparatus of any of Examples 57-65, further comprising: wherein the generator includes light-emitting diodes; a receptacle configured for receiving a probe connector housing ends of optical fibers; a latch; and a motor configured for aligning each of the ends of the optical fibers with a respective one of the light-emitting diodes by causing the latch to engage the probe connector.

Example 67 includes the apparatus of Example 66, wherein the motor is configured for releasing the probe connector for removal from the receptacle by causing the latch to disengage the connector.

Example 68 includes the apparatus of any of Examples 57-67, further comprising: wherein the generator includes light-emitting diodes; and a receptacle configured for receiving a probe connector housing ends of optical fibers; a latch; a first sensor configured for detecting the probe connector in the receptacle; and a motor configured for aligning each of the ends of the optical fibers with a respective one of the light-emitting diodes by causing the latch to engage the connector in response to the sensor detecting the connector in the receptacle.

Example 69 includes the apparatus of Example 68, wherein the motor is configured for releasing the probe connector for removal from the receptacle by causing the latch to disengage the probe connector in response to a second sensor.

Example 70 includes the apparatus of Example 69, wherein the second sensor includes an electronic switch.

Example 71 includes the apparatus of any of Examples 57-70, further comprising: wherein the generator includes light-emitting diodes; a receptacle having contact regions and configured to receive a probe connector housing ends of optical fibers and including a latch-engagement region; a latch; and a motor configured for aligning each of the ends of the optical fibers with a respective one of the light-emitting diodes by causing the latch to engage the latch-engagement region to force the probe connector against the contact regions.

Example 72 includes the apparatus of Example 71, wherein the motor is further configured for releasing the probe connector for removal from the receptacle by causing the latch to disengage the latch-engagement region.

Example 73 includes the apparatus of any of Examples 57-72, further comprising a spectrometer configured: for receiving, from a probe, the redirected portion of the electromagnetic energy; and for generating, for each of at least one wavelength range in the redirected portion of the electromagnetic energy, a respective electronic signal related to a value of a characteristic of the at least one wavelength range.

Example 74 includes the apparatus of any of Examples 57-73, further comprising a spectrometer configured: for receiving, from a probe, the redirected portion of the electromagnetic energy; and for generating, for each of at least one wavelength range in the redirected portion of the electromagnetic energy, a respective electrical signal related to a combined intensity of one or more wavelengths present in the at least one wavelength range.

Example 75 includes the apparatus of any of Examples 57-74, further comprising: wherein the generator includes light-emitting diodes; a spectrometer having an input configured for receiving, from a probe, the redirected portion of the electromagnetic energy, and configured for generating, for each of at least one wavelength range in the redirected portion of the electromagnetic energy, a respective electronic signal related to a value of a characteristic of the at least one wavelength range; and a receptacle configured for receiving a probe connector housing ends of optical fibers, for aligning each of the ends of some of the optical fibers with a respective one of the light-emitting diodes, and for aligning each of the ends of at least one other of the optical fibers with the spectrometer input.

Example 76 includes the apparatus of any of Examples 57-75, further comprising: wherein the generator includes light-emitting diodes; a spectrometer having an input configured for receiving, from a probe, the redirected portion of the electromagnetic energy, and configured for generating, for each of at least one wavelength range in the redirected portion of the electromagnetic energy, a respective electronic signal related to a value of a characteristic of the at least one wavelength range; and a receptacle configured for receiving a probe connector housing ends of optical fibers, configured for aligning each of the ends of ones of the optical fibers with a respective one of the light-emitting diodes, configured for aligning each of the ends of at least one other of the optical fibers with the spectrometer input, and including an electromagnetic-radiation shield configured for disposal between the ones of the optical fibers while respective aligned with the light-emitting diodes and the at least one other of the optical fibers while aligned with the spectrometer input.

Example 77 includes the apparatus of any of Examples 57-76, further comprising: wherein the generator includes light-emitting diodes; a spectrometer having an input configured for receiving, from a probe, the redirected portion of the electromagnetic energy, and configured for generating, for each of at least one wavelength range in the redirected portion of the electromagnetic energy, a respective electronic signal related to a value of a characteristic of the at least one wavelength range; and a receptacle configured for receiving a probe connector housing ends of optical fibers and having a slot between a first set of the optical fibers and a second set of at least one of the optical fibers, configured for aligning each of the ends of the optical fibers of the first set with a respective one of the light-emitting diodes, configured for aligning each of the ends of the at least one optical fiber of the second set with the spectrometer input, and including an electromagnetic-radiation shield configured for disposal in the slot.

Example 78 includes the apparatus of any of Examples 57-77, wherein the computing circuit is configured to control the generator.

Example 79 includes the apparatus of any of Examples 57-78, wherein the computing circuit is configured for determining a level of oxygenation of one or more of the at least one cell in response to a respective value of a characteristic of each of at least one wavelength range of the portion of redirected electromagnetic energy.

Example 80 includes the apparatus of any of Examples 57-79, wherein the computing circuit is configured: for implementing a machine-learning algorithm; and for determining a level of oxygenation of one or more of the at least one muscle cell by providing, as at least one input to the implemented machine-learning algorithm, a respective value of a characteristic of each of at least one wavelength range of the portion of redirected electromagnetic energy.

Example 81 includes the apparatus of any of Examples 57-80, wherein the computing circuit is configured: for implementing a mathematical algorithm; and for determining a level of oxygenation of one or more of the at least one muscle cell by providing, as at least one input to the implemented mathematical algorithm, a respective value of a characteristic of each of at least one wavelength range of the portion of redirected electromagnetic energy.

Example 82 includes the apparatus of any of Examples 57-81, wherein the computing circuit is configured for implementing a mathematical model; and for determining a level of oxygenation of one or more of the at least one muscle cell by providing, as at least one input to the implemented mathematical model, a respective value of a characteristic of each of at least one wavelength range of the portion of redirected electromagnetic energy.

Example 83 includes the apparatus of any of Examples 57-82, wherein the computing circuit is configured: for implementing a locally weighted regression model; and for determining a level of oxygenation of one or more of the at least one muscle cell by providing, as at least one input to the implemented locally weighted regression model, a respective value of a characteristic of each of at least one wavelength range of the portion of redirected electromagnetic energy.

Example 84 includes a method, comprising: generating, during an oxygenation-determining time, electromagnetic energy including wavelengths within an approximate range of 400 nm-900 nm; and determining, in response to a portion of the electromagnetic energy redirected, during the oxygenation-determining time, by a body having at least one muscle cell, a level of oxygenation of one or more of the at least one muscle cell.

Example 85 includes the method of Example 84, further comprising: generating, for each of at least one wavelength range in the redirected portion of the electromagnetic energy, a respective electronic signal related to at least one value of a characteristic of the at least one wavelength range; and determining the level of oxygenation in response to the at least one value of the characteristic.

Example 86 includes the method of any of Examples 84-85, further comprising: generating, for each of at least one wavelength range in the redirected portion of the electromagnetic energy, a respective electronic signal related to a combined intensity of one or more wavelengths in the at least one wavelength range; and determining the level of oxygenation in response to at least one of the combined intensity.

Example 87 includes the method of any of Examples 84-86, further comprising: generating, during a calibration time, electromagnetic energy; generating, for each of at least one wavelength range in a portion of the electromagnetic energy redirected by a probe, a respective electronic signal related to a value of a characteristic of the at least one wavelength range; and determining, in response to at least one of the each value, the level of oxygenation of one or more of the at least one muscle cell.

Example 88 includes the method of any of Examples 84-87, further comprising: generating, during a calibration time, electromagnetic energy; generating, for each of at least one wavelength range in a portion of the electromagnetic energy redirected by a calibrator, a respective electronic signal related to a value of a characteristic of the at least one wavelength range; and determining, in response to at least one of the each value, the level of oxygenation of one or more of the at least one muscle cell.

Example 89 includes the method of any of Examples 84-88, further comprising: generating, during a calibration time, electromagnetic energy; generating, for each of at least one wavelength range in a portion of the electromagnetic energy redirected by a calibrator during the calibration time, a respective electronic signal related to a value of a characteristic of the at least one wavelength range; and determining, in response to at least one of the each value, the level of oxygenation of one or more of the at least one muscle cell.

Example 90 includes the method of any of Examples 84-89, further comprising: generating, during a calibration time, electromagnetic energy; generating, for each of at least one wavelength range in a portion of the electromagnetic energy redirected by a calibrator of a probe, a respective electronic signal related to a value of a characteristic of the at least one wavelength range; and determining, in response to at least one of the each value, the level of oxygenation of one or more of the at least one muscle cell.

Example 91 includes the method of any of Examples 84-90, further comprising: generating, during a calibration time, electromagnetic energy; generating, for each of at least one wavelength range in a portion of the electromagnetic energy redirected by a calibrator, a respective electronic signal related to a value of a characteristic of the at least one wavelength range; storing at least one of the at least one value; and determining, in response to the stored at least one of the at least one value, the level of oxygenation of one or more of the at least one muscle cell.

Example 92 includes the method of any of Examples 84-91, further comprising: generating, for each of at least one wavelength range in the redirected portion of the electromagnetic energy, a respective electronic signal related to at least one value of a characteristic of the at least one wavelength range; generating, during a calibration time, electromagnetic energy; generating, for each of at least one wavelength range in a portion of the electromagnetic energy redirected by a calibrator, a respective electronic signal related to at least one calibration value of a characteristic of the at least one wavelength range; and determining, in response to a respective ratio of each of at least one of the at least one value and each of at least one of the at least one calibration value for a respective one of the at least one wavelength range, the level of oxygenation of one or more of the at least one muscle cell.

Example 93 includes the method of any of Examples 84-92, further comprising: Generating, for each of at least one wavelength range in the redirected portion of the electromagnetic energy, a respective electronic signal related to at least one value of a characteristic of the at least one wavelength range; generating, during a calibration time, electromagnetic energy; generating, for each of at least one wavelength range in a portion of the electromagnetic energy redirected by a calibrator, a respective electronic signal related to at least one calibration value of a characteristic of the at least one wavelength range; and determining, in response to a logarithm of a respective ratio of each of at least one of the at least one value and each of at least one of the at least one calibration value for a respective one of the at least one wavelength range, the level of oxygenation of one or more of the at least one muscle cell.

Example 94 includes a method, comprising: oxygenating cells in a body part; inducing an ischemia in the body part; returning normal blood flow to the body part; directing, toward the body part, electromagnetic energy including wavelengths in an approximate range of 400 nm-900 mn, for each of at least one first time during the oxygenating, inducing, and returning; generating, during each of the at least one first time for each of at least one wavelength range in a portion of the electromagnetic energy redirected by the body part, a respective value of a characteristic of the at least one wavelength range; and storing the at least one value.

Example 95 includes the method of Example 94, wherein oxygenating cells includes causing a subject having the body part to breathe in oxygen in a concentration greater than a concentration of oxygen in air for a second time that is greater than the at least one first time.

Example 96 includes the method of any of Examples 94-95, wherein inducing an ischemia includes reducing blood flow to the body part.

Example 97 includes the method of any of Examples 94-96, wherein inducing an ischemia includes inflating a cuff disposed around the body part.

Example 98 includes the method of any of Examples 94-97, wherein: inducing an ischemia includes inflating a cuff disposed around the body part; and returning normal blood flow includes deflating the cuff.

Example 99 includes the method of any of Examples 94-98, wherein the body part includes a thenar eminence of a human hand.

Example 100 includes the method of any of Examples 94-99, wherein directing the electromagnetic energy includes: generating the electromagnetic energy with at least one light-emitting diode; and directing the electromagnetic energy from each of the at least one light-emitting diode toward the body part with a respective optical fiber.

Example 101 includes the method of any of Examples 94-100, wherein generating includes: collecting, with at least one optical fiber, the redirected portion of the electromagnetic energy; and generating, during each of the at least one first time for each of the at least one wavelength range, the respective value using a spectrometer.

Example 102 includes the method of any of Examples 94-101, further comprising training an algorithm using the stored at least one value of the characteristic.

Example 103 includes the method of any of Examples 94-102, further comprising training a locally weighted regression model using the stored at least one value of the characteristic.

Example 104 includes the method of any of Examples 94-103, further comprising training a neural network using the stored at least one value of the characteristic.

Example 105 includes the method of any of Examples 94-104, further comprising training a convolutional neural network using the stored at least one value of the characteristic.

Example 106 includes the method of any of Examples 94-105, further comprising training a machine-learning algorithm using the stored at least one value of the characteristic.

Example 107 includes the method of any of Examples 94-106, further comprising training a statistical-learning algorithm using the stored at least one value of the characteristic.

Example 108 includes the method of any of Examples 94-107, further comprising training a support-vector-machine algorithm using the stored at least one value of the characteristic.

Example 109 includes the method of any of Examples 94-108, further comprising training a decision-tree-method algorithm using the stored at least one value of the characteristic.

Example 110 includes the method of any of Examples 94-109, further comprising training a random-forest algorithm using the stored at least one value of the characteristic.

Example 111 includes the method of any of Examples 94-110, further comprising training an XGBoost algorithm using the stored at least one value of the characteristic.

Example 112 includes the method of any of Examples 94-111, further comprising training a feed-forward neural network using the stored at least one value of the characteristic.

Example 113 includes the method of any of Examples 94-112, further comprising training a recurrent neural network using the stored at least one value of the characteristic.

Example 114 includes the method of any of Examples 94-113, further comprising training a generative-adversarial neural network using the stored at least one value of the characteristic.

Example 115 includes the method of any of Examples 94-114, further comprising training a recommender-system algorithm using the stored at least one value of the characteristic.

Example 116 includes a program product comprising a non-transitory processor-readable medium on which program instructions configured to be executed by at least one processor are embodied, wherein when executed by the at least one processor, the program instructions cause the at least one processor to: generate, during an oxygenation-determining time, electromagnetic energy including wavelengths within an approximate range of 400 nm-900 nm; and determine, in response to a portion of the electromagnetic energy redirected, during the oxygenation-determining time, by a body having at least one muscle cell, a level of oxygenation of one or more of the at least one muscle cell.

Example 117 includes the program product of Example 116, wherein the program instructions cause the at least one processor to: generate, for each of at least one wavelength range in the redirected portion of the electromagnetic energy, a respective electronic signal related to a combined intensity of one or more wavelengths in the at least one wavelength range; and determine the level of oxygenation in response to at least one of the combined intensity.

Example 118 includes the program product of any of Examples 116-117, wherein the program instructions cause the at least one processor to: generate, for each of at least one wavelength range in a portion of the electromagnetic energy redirected by a probe, a respective electronic signal related to a value of a characteristic of the at least one wavelength range; and determine, in response to at least one of the at least one value, the level of oxygenation of one or more of the at least one muscle cell.

Example 119 includes the program product of any of Examples 116-118, wherein the program instructions cause the at least one processor to: generate, for each of at least one wavelength range in a portion of the electromagnetic energy redirected by a calibrator, a respective electronic signal related to a value of a characteristic of the at least one wavelength range; and determine, in response to at least one of the value, the level of oxygenation of one or more of the at least one cell.

Example 120 includes the program product of any of Examples 116-119, wherein the program instructions cause the at least one processor to: generate, for each of at least one wavelength range in a portion of the electromagnetic energy redirected by a calibrator during a calibration time, a respective electronic signal related to at least one value of a characteristic of the at least one wavelength range; and determine, in response to at least one of the at least one value, the level of oxygenation of one or more of the at least one muscle cell.

Example 121 includes the program product of any of Examples 116-120, wherein the program instructions cause the at least one processor to: generate, for each of at least one wavelength range in a portion of the electromagnetic energy redirected by a calibrator of a probe, a respective electronic signal related to at least one value of a characteristic of the at least one wavelength range; and determine, in response to at least one of the at least one value, the level of oxygenation of one or more of the at least one muscle cell.

Example 122 includes the program product of any of Examples 116-121, wherein the program instructions cause the at least one processor to: generate, for each of at least one wavelength range in a portion of the electromagnetic energy redirected by a calibrator, a respective electronic signal related to at least one value of a characteristic of the at least one wavelength range; store at least one of the at least one value; and determine, in response to the stored at least one of the at least one value, the level of oxygenation of one or more of the at least one muscle cell.

Example 123 includes the program product of any of Examples 116-122, wherein the program instructions cause the at least one processor to: generate, for each of at least one wavelength range in a portion of the electromagnetic energy redirected by a calibrator, a respective electronic signal related to a calibration value of a characteristic of the at least one wavelength range; and determine, in response to a respective ratio of each of at least one of the at least one value and each of at least one of the at least one calibration value for a respective one of the at least one wavelength range, the level of oxygenation of one or more of the at least one muscle cell.

Example 124 includes the program product of any of Examples 116-123, wherein the program instructions cause the at least one processor to: generate, for each of at least one wavelength range in a portion of the electromagnetic energy redirected by a calibrator, a respective electronic signal related to at least one calibration value of a characteristic of the at least one wavelength range; and determine, in response to a logarithm of a respective ratio of each of at least one of the at least one value and each of at least one of the at least one calibration value for a respective one of the at least one wavelength range, the level of oxygenation of one or more of the at least one muscle cell.

Example 125 includes an apparatus, comprising: means for generating, during an oxygenation-determining time, electromagnetic energy including wavelengths within an approximate range of 400 nm-900 nm; and means for determining, in response to a portion of the electromagnetic energy redirected, during the oxygenation-determining time, by a body having at least one muscle cell, a level of oxygenation of one or more of the at least one muscle cell.

Example 126 includes the apparatus of Example 125, further comprising: means for generating, for each of at least one wavelength range in the redirected portion of the electromagnetic energy, a respective electronic signal related to at least one value of a characteristic of the at least one wavelength range; and means for determining the level of oxygenation in response to the at least one value of the characteristic.

Example 127 includes the apparatus of any of Examples 125-126, further comprising: means for generating, for each of at least one wavelength range in the redirected portion of the electromagnetic energy, a respective electronic signal related to a combined intensity of one or more wavelengths in the at least one wavelength range; and means for determining the level of oxygenation in response to at least one of the combined intensity.

Example 128 includes the apparatus of any of Examples 125-127, further comprising: means for generating, during a calibration time, electromagnetic energy; means for generating, for each of at least one wavelength range in a portion of the electromagnetic energy redirected by a probe, a respective electronic signal related to a value of a characteristic of the at least one wavelength range; and means for determining, in response to at least one of the each value, the level of oxygenation of one or more of the at least one muscle cell.

Example 129 includes the apparatus of any of Examples 125-128, further comprising: means for generating, during a calibration time, electromagnetic energy; means for generating, for each of at least one wavelength range in a portion of the electromagnetic energy redirected by a calibrator, a respective electronic signal related to a value of a characteristic of the at least one wavelength range; and means for determining, in response to at least one of the each value, the level of oxygenation of one or more of the at least one muscle cell.

Example 130 includes the apparatus of any of Examples 125-129, further comprising: means for generating, during a calibration time, electromagnetic energy; means for generating, for each of at least one wavelength range in a portion of the electromagnetic energy redirected by a calibrator during the calibration time, a respective electronic signal related to a value of a characteristic of the at least one wavelength range; and means for determining, in response to at least one of the each value, the level of oxygenation of one or more of the at least one muscle cell.

Example 131 includes the apparatus of any of Examples 125-130, further comprising: means for generating, during a calibration time, electromagnetic energy; means for generating, for each of at least one wavelength range in a portion of the electromagnetic energy redirected by a calibrator of a probe, a respective electronic signal related to a value of a characteristic of the at least one wavelength range; and means for determining, in response to at least one of the each value, the level of oxygenation of one or more of the at least one muscle cell.

Example 132 includes the apparatus of any of Examples 125-131, further comprising: means for generating, during a calibration time, electromagnetic energy; means for generating, for each of at least one wavelength range in a portion of the electromagnetic energy redirected by a calibrator, a respective electronic signal related to a value of a characteristic of the at least one wavelength range; means for storing at least one of the at least one value; and means for determining, in response to the stored at least one of the at least one value, the level of oxygenation of one or more of the at least one muscle cell.

Example 133 includes the apparatus of any of Examples 125-132, further comprising: means for generating, for each of at least one wavelength range in the redirected portion of the electromagnetic energy, a respective electronic signal related to at least one value of a characteristic of the at least one wavelength range; means for generating, during a calibration time, electromagnetic energy; means for generating, for each of at least one wavelength range in a portion of the electromagnetic energy redirected by a calibrator, a respective electronic signal related to at least one calibration value of a characteristic of the at least one wavelength range; and means for determining, in response to a respective ratio of each of at least one of the at least one value and each of at least one of the at least one calibration value for a respective one of the at least one wavelength range, the level of oxygenation of one or more of the at least one muscle cell.

Example 134 includes the apparatus of any of Examples 125-133, further comprising: means for generating, for each of at least one wavelength range in the redirected portion of the electromagnetic energy, a respective electronic signal related to at least one value of a characteristic of the at least one wavelength range; means for generating, during a calibration time, electromagnetic energy; means for generating, for each of at least one wavelength range in a portion of the electromagnetic energy redirected by a calibrator, a respective electronic signal related to at least one calibration value of a characteristic of the at least one wavelength range; and means for determining, in response to a logarithm of a respective ratio of each of at least one of the at least one value and each of at least one of the at least one calibration value for a respective one of the at least one wavelength range, the level of oxygenation of one or more of the at least one muscle cell.

From the foregoing it will be appreciated that, although specific embodiments have been described herein for purposes of illustration, various modifications may be made without deviating from the spirit and scope of the disclosure. Furthermore, where an alternative is disclosed for a particular embodiment, this alternative may also apply to other embodiments even if not specifically stated. Moreover, one or more components of a described apparatus or system, or one or more steps of a described method, may have been omitted from the description for clarity or for another reason. In addition, one or more components of a described apparatus or system that have been included in the description may be omitted from the apparatus or system, and one or more steps of a described method that have been included in the description may be omitted from the method.

1. An apparatus, comprising:
a broadband electromagnetic-energy source;
electromagnetic-energy sensors;
a first optical fiber having a first end disposable adjacent to a biological subject and having a second end disposable adjacent to the broadband electromagnetic-energy source;
a second optical fiber having a first end disposable adjacent to the biological subject and having a second end disposable adjacent to the electromagnetic-energy sensors; and
an electronic circuit configured to determine, according to a mathematical algorithm, a level of oxygen in cells of the biological subject in response to the electromagnetic-energy sensors.

2. The apparatus of claim 1 wherein:
the first end of the second optical fiber is configured to receive first electromagnetic energy from the biological subject;
the second end of the second optical fiber is configured to irradiate the electromagnetic-energy sensors with the second electromagnetic energy that is related to the first electromagnetic energy; and
the electromagnetic-energy sensors are configured to determine intensities of bands of wavelengths of the second electromagnetic energy.

3. The apparatus of claim 1 wherein the electromagnetic-energy sensors form at least part of a spectrometer.

4. The apparatus of claim 3 wherein:
the first end of the second optical fiber is configured to receive first electromagnetic energy from the biological subject;
the second end of the second optical fiber is configured to irradiate the spectrometer with the second electromagnetic energy that is related to the first electromagnetic energy; and
the spectrometer is configured to determine intensities of bands of wavelengths of the second electromagnetic energy.

5. The apparatus of claim 1 wherein the electromagnetic-energy sensors form at least part of an optical spectrometer.

6. The apparatus of claim 5 wherein:
the first end of the second optical fiber is configured to receive first electromagnetic energy from the biological subject;
the second end of the second optical fiber is configured to irradiate the spectrometer with the second electromagnetic energy that is related to the first electromagnetic energy; and
the optical spectrometer is configured to determine intensities of bands of wavelengths of the second electromagnetic energy.

7. The apparatus of claim 1 wherein the mathematical algorithm includes a learning algorithm.

8. The apparatus of claim 1 wherein the cells include muscle cells.

9. The apparatus of claim 1 wherein the cells include blood cells.

10. The apparatus of claim 1 wherein the broadband electromagnetic-energy source is configured to generate wavelengths in a range of approximately 400 nm-1000 nm.

11. An apparatus, comprising:
a source configured to irradiate a biological subject with broadband electromagnetic energy;
electromagnetic-energy sensors configured to generate signals in response to bands of a portion of the broadband electromagnetic energy redirected by the biological subject; and
an electronic circuit configured to determine, according to a mathematical algorithm and in response to the signals, a level of oxygen in cells of the biological subject.

12. The apparatus of claim 11 wherein the source includes at least one broadband light-emitting diode.

13. The apparatus of claim 11 wherein the source includes a polychromatic light source.

14. The apparatus of claim 11 wherein the electromagnetic-energy sensors are configured to generate the signals in response to bands of wavelengths of the portion of the broadband electromagnetic energy redirected by the biological subject.

15. The apparatus of claim 11 wherein the electromagnetic-energy sensors are configured to generate the signals in response to intensities of bands of wavelengths of the portion of the broadband electromagnetic energy redirected by the biological subject.

16. The apparatus of claim 11 wherein the electromagnetic-energy sensors form at least part of a spectrometer.

17. The apparatus of claim 11 wherein the mathematical algorithm includes a learning algorithm.

18. The apparatus of claim 11 wherein the cells include muscle cells.

19. The apparatus of claim 11 wherein the cells include blood cells.

20. The apparatus of claim 11, further comprising at least one optical fiber having a first end configured to receive broadband electromagnetic energy from the source and having a second end configured to irradiate the biological subject with the broadband electromagnetic energy.

21. The apparatus of claim 11, further comprising at least one optical fiber having a first end configured to receive broadband electromagnetic energy redirected by the biological subject and having a second end configured to irradiate the electromagnetic-energy sensors with the portion of the broadband electromagnetic energy redirected by the biological subject.

22. The apparatus of claim 11, further comprising:
a housing in which are disposed the source, electromagnetic-energy sensors, and electronic circuit; and
a member configured to attach the housing to the biological subject.

23. A method, comprising:
irradiating a biological subject with broadband electromagnetic energy;
sensing wavelength bands of a portion of the broadband electromagnetic energy redirected by the biological subject;
generating signals in response to the sensed wavelength bands; and
determining, with a mathematical algorithm and in response to the signals, a level of oxygen in cells of the biological subject.

24. The method of claim 23 wherein irradiating the biological subject includes directing the broadband electromagnetic energy toward the biological subject with at least one optical fiber.

25. The method of claim 23 wherein sensing wavelength bands includes directing the portion of the broadband electromagnetic energy toward electromagnetic-energy sensors with at least one optical fiber.

26. The method of claim 23 wherein generating signals includes generating the signals with electromagnetic-energy sensors.

27. The method of claim 23 wherein determining a level of oxygen includes determining the level of oxygen in the cells of the biological subject using an electronic circuit implementing the mathematical algorithm.

28. The method of claim 23 wherein sensing wavelength bands includes sensing intensities of the wavelength bands.

29. A tangible computer-readable medium storing instructions that, when executed, cause a processing circuit, or another electronic circuit coupled to the processing circuit:
to irradiate a biological subject with broadband electromagnetic energy;
to sense wavelength bands of a portion of the broadband electromagnetic energy redirected by the biological subject;
to generate signals in response to the sensed wavelength bands; and
to determine, with a mathematical algorithm and in response to the signals, a level of oxygen in cells of the biological subject.

30. A tangible medium storing configuration data that, when used to configure an electronic circuit, cause the electronic circuit, or another electronic circuit coupled to the electronic circuit:
to irradiate a biological subject with broadband electromagnetic energy;
to sense wavelength bands of a portion of the broadband electromagnetic energy redirected by the biological subject;
to generate signals in response to the sensed wavelength bands; and
to determine, with a mathematical algorithm and in response to the signals, a level of oxygen in cells of the biological subject.
